(19) United States
(12) Patent Application Publication
(10) Pub. No.: US 2013/0177598 A1
(43) Pub. Date: Jul. 11, 2013

(54) DISCRETE SIZE AND SHAPE SPECIFIC PHARMACEUTICAL ORGANIC NANOPARTICLES

(71) Applicants: Joseph M. Desimone, Chapel Hill, NC (US); Stephanie Gratton, Chapel Hill, NC (US); Ji Guo, Rockville, MD (US); Jennifer Yvonne Kelly, Chapel Hill, NC (US); Andrew James Murphy, Chapel Hill, NC (US); Mary E Napier, Carrboro, NC (US)

(72) Inventors: Joseph M. Desimone, Chapel Hill, NC (US); Stephanie Gratton, Chapel Hill, NC (US); Ji Guo, Rockville, MD (US); Jennifer Yvonne Kelly, Chapel Hill, NC (US); Andrew James Murphy, Chapel Hill, NC (US); Mary E Napier, Carrboro, NC (US)

(73) Assignee: The University of North Carolina at Chapel Hill, Chapel Hill, NC (US)

(21) Appl. No.: 12/528,571

(22) PCT Filed: Feb. 27, 2008
(86) PCT No.: PCT/US08/55109

§ 371 Date: Apr. 29, 2010

Related U.S. Application Data
(60) Provisional application No. 60/903,719, filed on Feb. 27, 2007.

(51) Int. Cl. A61K 9/14

(52) U.S. Cl. CPC A61K 9/14
USPC 424/400; 514/449; 514/44 A; 514/34; 514/291; 424/94.1; 424/94.63; 264/4.3; 428/402

(57) ABSTRACT

A pharmaceutical composition comprising protein micro and/or nanoparticles are provided. The particles have a predetermined geometric shape and a broadest dimension less than about 10 micrometers. The particles may further comprise active agents.

### FEDERALLY SPONSORED RESEARCH OR DEVELOPMENT

This invention was made with U.S. Government support from the Science & Technology Center for Environmentally Responsible Solvents and Processes program of the National Science Foundation under Agreement No. CHE-9876674 and the Carolina Center for Nanotechnology Excellence program of the National Institutes of Health under No. 5-654-CA119373-02. The U.S. Government has certain rights in the invention.

### TECHNICAL FIELD OF THE INVENTION

Generally, the present invention relates to therapeutic nanoparticles. More particularly, the therapeutic nanoparticles are size and shape specific and have a composition that includes a protein or a protein in combination with an active agent.

### BACKGROUND OF THE INVENTION

It has been reported that 1 in 10 marketed drugs have solubility problems, over a third of pipeline drugs are poorly soluble, and almost ⅔ of drugs coming from early pre-clinical development have low solubility. As such, almost 40% of all possible drug targets fail early due to poor solubility characteristics and insufficient pharmacokinetics. Biological agents, such as siRNA have a short half-life and are easily degraded. Due to these factors, the agents need to be protected during circulation, delivered to the desired tissue and then released intra-cellularly into the cytosol to be used effectively as a therapeutic. Proteins and peptides face similar hurdles and in addition, they can also trigger an immunological response. The delivery of these therapeutic agents, as well as the delivery of detection and imaging agents for the diagnosis and treatment of disease, has improved somewhat over the years with the development of nano-carriers such as liposomes, micelles, dendrimers, polymer particles, polymer conjugates and colloidal precipitates. However, only a handful of drugs and imaging agents delivered using these approaches have success in treating patients in the clinic.

It is known that colloidal nanoparticles or particles <200 nm in size tend to concentrate at the tumor site due to leaky vasculatures. Therefore, it is possible that localized nanoparticles containing an anticancer formulation will aggregate at the tumor site, thereby resulting in greater efficacy when compared to typical drug administration.

However, current nanoparticle drug products have several drawbacks. First, prior to delivery the current product must be reconstituted and after being reconstituted the product has a relatively short shelf life. Furthermore, the manufacturing processes for forming the current product are complicated and can result in either damage to the protein or a limitation of what proteins can be utilized in the current product.

An ideal therapeutic carrier would be biocompatible, shape and size specific, monodisperse, composed of virtually any material, amenable to functionalization, and gentle enough for fragile biological cargo.

### SUMMARY OF THE INVENTION

Compositions and methods for the delivery of active agents are provided. The compositions comprise shape-specific protein micro and/or nanoparticles having a polydispersity of 0.0-0.08. The protein micro and/or nanoparticles may additionally comprise at least one active agent. The protein micro and/or nanoparticles may also comprise inactive ingredients. The compositions are useful for the delivery of any active agent including proteins, small molecules, pharmaceuticals, nucleotide sequences such as DNA, RNA, and siRNA, imaging agents, and the like. The compositions may be formulated to provide targeting to specific cells or tissues of interest.

Using methods of the invention, protein micro and/or nanoparticles (also referred to herein as pharmaceutical organic particles) can be formulated into a discrete size and shape. These protein micro and/or nanoparticles can be formulated into pharmaceutical compositions. In some embodiments, a protein micro and/or nanoparticle includes a particle having a predetermined geometric shape and a broadest dimension less than about 10 micrometers and where the particle composition includes at least one protein or polypeptide that is an active agent or an active pharmaceutical agent and a protein. That is, by the methods of the invention, proteins or polypeptides can be molded into nanoparticles and such particles used in therapeutic or diagnostic methods of the invention. Additionally, the protein micro and/or nanoparticle may comprise at least one active agent. The pharmaceutical composition can also include a plurality of particles, where each particle of the plurality of particles is substantially the same size and has substantially the same geometric shape. In some embodiments, the particles of the plurality of particles have a polydispersity of about 0.003. A number of proteins or polypeptides can be used to form the protein micro and/or nanoparticle. In alternative embodiments, the active pharmaceutical agent is hydrophobic, hydrophilic, or a biologic.

In some embodiments, the protein component is a matrix of the particle. In alternative embodiments, the protein is crosslinked, crosslinked with disulfide bonds, crosslinked by sonication in a non-solvent, crosslinked in a polar non-solvent such as chloroform, crosslinked by humidity, or crosslinked thermally. In some embodiments, the particle is substantially a 200 nm diameter by 200 nm tall cylinder. According to alternative embodiments, the particle is configured to controllably degrade, degrade after exposure to a predetermined environment, or degrade after exposure to a predetermined environment for a predetermined quantity of time.

In other embodiments of the present invention, the pharmaceutical composition comprises a plurality of particles without a solvent or solution. In some embodiments the protein is configured to solubilize the active hydrophobic pharmaceutical agent. In other embodiments the protein is configured to shield the active hydrophobic pharmaceutical agent from degradation.

According to alternative embodiments an organic particle of the present invention includes a particle having a predetermined geometric shape, a broadest dimension less than about 10 micrometers, and a composition of a protein.

### BRIEF DESCRIPTION OF THE FIGURES

FIGS. 1A-1D show fabrication of molded nanoparticles according to an embodiment of the present invention;

FIGS. 2A-2E show formation of micro and/or nanoparticles according to an embodiment of the present invention;

FIGS. 3A-3F show yet further fabrication of micro and/or nanoparticles according to another embodiment of the present invention;

FIG. 4 shows a laminate mold having micro and/or nano sized cavities according to another embodiment of the present invention;

FIG. 5 shows formation of a laminate mold for use in the present invention;

FIGS. 6A-6E show reduction molding of micro and/or nanoparticles according to an embodiment of the present invention;

FIGS. 7A-7E show open molding of micro and/or nanoparticles according to an embodiment of the present invention;

FIGS. 8A-8F show harvesting of molded micro and/or nanoparticles according to an embodiment of the present invention;

FIGS. 9A-9F show harvesting of molded micro and/or nanoparticles according to another embodiment of the present invention;

FIG. 10 shows comparative polydispersity measurements of Abraxane® particles, liposomes, and micro and/or nanoparticles of the present invention;

FIG. 11 shows proposed compounds for crosslinking protein particles with degradable crosslinkers according to some embodiments of the present invention;

FIG. 12 shows alternate magnification SEM images of 200 nm tall×200 nm diameter cylindrical albumin particles according to an embodiment of the present invention;

FIG. 13 shows an SEM image of albumin particles on a medical adhesive layer according to an embodiment of the present invention;

FIG. 14 shows an SEM images of albumin 50 wt % PBS w/0.5 wt % siRNA on a medical adhesive layer according to an embodiment of the present invention;

FIG. 15 shows 200 nm×200 nm cylindrical patterned transferrin film according to an embodiment of the present invention; and

FIG. 16 shows 200 nm×200 nm cylindrical patterned transferrin film and free 200 nm×200 nm transferrin cylinders according to an embodiment of the present invention.

FIGS. 17A-17D show SEM micrographs of 200×200 nm Abraxane particles on medical adhesive, directly using chloroform; 200×600 nm Abraxane particles harvested directly using chloroform.

FIGS. 18A-18B show SEM micrographs of 200×200 nm interferon-beta particles on medical adhesive.

FIGS. 19A-19D show SEM micrographs of 200×200 nm (first three SEMS) and 2 micron, AR2 insulin particles on medical adhesive.

FIGS. 20A-20D show SEM micrographs of 5 μm transferrin particles on medical adhesive (first two images) and 200×200 nm cylinders of transferrin harvested directly using chloroform (third SEM) or harvested on medical adhesive (fourth SEM).

FIG. 21 shows an SEM micrograph of 5 μm albumin particles on medical adhesive.

FIG. 22 shows an SEM micrograph of 200×200 nm horse radish peroxidase particles harvested on Povidone.

FIG. 23 shows an SEM micrograph of 200×200 nm trypsin particles harvested on Povidone.

FIG. 24 shows an SEM micrograph of 200×200 nm hemoglobin particles harvested on Povidone.

FIGS. 25A-25B show Optical microscopy images (DIC) of 5 μm hemoglobin particles and the corresponding fluorescent image.

FIGS. 26A-26B show Confocal images of the above sample.

FIGS. 27A-27C show RhodamineB-loaded albumin particles harvested on Povidone to monitor dissolution in water. FIG. 27A shows the DIC and fluorescent image of 5 um albumin+dye PRINT particles. FIG. 27B shows the image after the addition of water. FIG. 27C shows the image after complete dissolution of particles.

FIG. 28 shows SEM micrographs of 200×200 nm albumin particles loaded with ethylene glycol-coated gadolinium oxide.

FIG. 29 shows dynamic light scattering of Abraxane molded using PRINT and reconstituted Abraxane.

FIG. 30 shows the ELISA assay on free albumin vs albumin PRINT.

FIG. 31 shows SEM micrographs of 200×200 nm IgG particles harvested on medical adhesive.

FIG. 32 shows the enzyme activity assay results for free horseradish peroxidase vs horseradish peroxidase PRINT.

### DETAILED DESCRIPTION OF EMBODIMENTS OF THE INVENTION

The present subject matter will now be described more fully hereinafter with reference to the accompanying Figures and Examples, in which representative embodiments are shown. The present subject matter can, however, be embodied in different forms and should not be construed as limited to the embodiments set forth herein. Rather, these embodiments are provided to describe and enable one of skill in the art to practice the present invention. Unless otherwise defined, all technical and scientific terms used herein have the same meaning as commonly understood by one of ordinary skill in the art to which the subject matter belongs. All publications, patent applications, patents, and other references mentioned herein are incorporated by reference in their entirety. Furthermore, throughout the specification and claims a given chemical formula or name shall encompass all optical and stereoisomers, as well as racemic mixtures where such isomers and mixtures exist and all pharmaceutical compositions include any and all pharmaceutically acceptable salt thereof. For a review on pharmaceutically acceptable salts see Berge et al., 66 J. Pharm. Sci. 1-19 (1977), which is incorporated herein by reference.

Compositions for the delivery of active agents are provided. The compositions comprise shape-specific protein micro and/or nanoparticles having a polydispersity of 0.0-0.08. The particles may additionally contain at least one active agent. The nanoparticles of the invention provide independent control over variables such as size, shape, composition, cargo encapsulation, surface functionality, and biodistribution. The protein micro and/or nanoparticles of the invention are formed of proteins or polypeptides. These proteins or polypeptides may be an active agent protein or alternatively the proteins or polypeptides may be carriers for at least one active agent. Both the active agent protein and the carrier protein can be formulated with at least one active or pharmaceutical agent. Additionally, other carriers or ingredients may be utilized.

A structural component of the micro and/or nanoparticles of the invention is protein. Depending upon the protein used in the making of the particle and the therapeutic or diagnostic use, the protein component of the micro and/or nanoparticles will vary. Generally, at least about 20%, about 25%, about 30%, about 40%, about 50%, about 60%, about 70%, about 80%, about 90% up to about 100% by weight, (w/w) of the particle is protein.

The protein may be combined with buffers or other excipients prior to formation of the particles. Other acceptable components in the protein micro and/or nanoparticles of the invention include, but are not limited to, pharmaceutically acceptable agents including water, salts, sugars, polyols, amino acids, and buffers. Examples of suitable buffers include phosphate, citrate, succinate, acetate, and other organic acids or their salts and salts such as sodium chloride, sodium phosphate, sodium sulfate, potassium chloride. Thus, the protein component of the particles of the invention can be formulated with a pharmaceutically acceptable buffer, including, for example, conventional buffers of organic acids and salts thereof such as citrate buffers (e.g., monosodium citrate-disodium citrate mixture, citric acid-trisodium citrate mixture, citric acid-monosodium citrate mixture, etc.), succinate buffers (e.g., succinic acid-monosodium succinate mixture, succinic acid-sodium hydroxide mixture, succinic acid-disodium succinate mixture, etc.), tartrate buffers (e.g., tartaric acid-sodium tartrate mixture, tartaric acid-potassium tartrate mixture, tartaric acid-sodium hydroxide mixture, etc.), fumarate buffers (e.g., fumaric acid-monosodium fumarate mixture, fumaric acid-disodium fumarate mixture, monosodium fumarate-disodium fumarate mixture, etc.), gluconate buffers (e.g., gluconic acid-sodium gluconate mixture, gluconic acid-sodium hydroxide mixture, gluconic acid-potassium gluconate mixture, etc.), oxalate buffers (e.g., oxalic acid-sodium oxalate mixture, oxalic acid-sodium hydroxide mixture, oxalic acid-potassium oxalate mixture, etc.), lactate buffers (e.g., lactic acid-sodium lactate mixture, lactic acid-sodium hydroxide mixture, lactic acid-potassium lactate mixture, etc.), phosphate buffers (sodium phosphate monobasic/sodium phosphate dibasic), and acetate buffers (e.g., acetic acid-sodium acetate mixture, acetic acid-sodium hydroxide mixture, etc.). Other excipients such as trehalose, mannose, sucrose and the like may also be used. Thus, by manipulating the protein concentration within the solution, a particle may be formed having the desired % weight of protein.

The micro and/or nanoparticles of the invention have a polydispersity of 0-0.08. In some embodiments the particles are monodisperse. By “monodisperse” is intended that the particles are of a uniform size. That is, the particles have a polydispersity of 0-0.02 as measured by Cumulant Analysis (D. Koppel (1972) J. Chem. Phys. 57:4814, herein incorporated by referenced. For calculation of polydispersity see for example, Section IV of the present specification as well as Example 4.1.

By “active agent protein” is intended a therapeutic or diagnostic protein that is used to form the protein micro and/or nanoparticle. The protein micro and/or nanoparticle formed using an active agent protein can be used as a therapeutic or diagnostic nanoparticle without the need for additional active agents. However, it is recognized that one or more active agents may be included with the active agent protein to form the micro and/or nanoparticles. That is, more than one therapeutic protein can be used to form the particles.

Active agent proteins include insulin, interferon (interferon-α and interferon-β), transferrin, protein C, hirudin, granulocyte-macrophage colony-stimulating factor, somatropin, epidermal growth factor, albumin, hemoglobin, lactoferrin, angiotensin-converting enzyme, glucocerebrosidase, human growth hormone, VEGF, GLP-1, gentamycin and Amikacin, therapeutic peptides and proteins, such as EPO, G-CSF, GM-CSF, Factor VIR, LHRH analogues and interferons, other biopharmaceuticals, such as heparin, and vaccines, such as Hepatitis ‘B’ surface antigen, typhoid, and cholera, proteins naturally produced by the human body, recombinant versions of such proteins, and derivatives and analogs of such proteins, and soluble portions of receptors. For example, cytokines such as interleukins and interferons, and growth factors and soluble fractions of cytokine and growth factor receptors may be used. Cytokines are often low molecular weight glycoproteins. Proteins of interest also include, without limitation, enzymes, growth factors, monoclonal antibody, antibody fragments, single-chain antibody, immunoglobulins, clotting factors, amylase, lipase, protease, cellulose, urokinase, galactosidase, staphylokinase, hyaluronidase, tissue plasminogen activator, and the like. Active agent proteins can include monoclonal antibodies, for example abciximab, adalimumab, alemtuzumab, basiliximab, bevacizumab, cetuximab, daclizumab, eculizumab, efalizumab, ibritumomab tiuxetan, infliximab, muromonab-CD3, natalizumab, omalizumab, palivizumab, panitumumab, ranibizumab, rituximab, traztuzumab, etc.

Essentially any therapeutic protein can be used in the manufacture of the protein micro and/or nanoparticles as the active agent protein as long as the protein is capable of dissolving in a solution.

By “carrier” is intended a protein or polypeptide that has little or no therapeutic activity but is useful as a transport vehicle for an active agent. Such carrier proteins include albumin, modified albumin, hemoglobin, growth factor binding proteins, calcium binding proteins, acyl carrier proteins, and the like. In some instances conformationally modified albumins (albumin-Au, formaldehyde-, maleic anhydride-treated albumin) may be used. Such albumins bind preferentially with a greater affinity to albumin-binding proteins on the endothelial cell surface and can be used to target the active agent to cells of interest, particularly cancer cells, more particularly breast cancer cells. See, for example, Schnitzer, and Bravo (1993) J. Biol. Chem. 268:7562-7570; and Schnitzer et al. (1992) J. Biol. Chem. 267:24544-24553; herein incorporated by reference. It is recognized that a synthetic polypeptide can be used as a carrier protein. Synthetic polypeptides include polypeptides designed as carrier proteins, derivatives and variants of therapeutic proteins that have an altered amino acid sequence to decrease or eliminate activity yet maintain structural integrity for use as a carrier protein. Therapeutic proteins that have been altered to provide linkage to a targeting moiety may lose their activity yet find use as carriers. Carriers may provide cavities or associations where the active agent may be suspended or associated with each carrier molecule.

As indicated the active agent protein and the carrier protein may be formulated with at least one active agent to form the protein micro and/or nanoparticles. While theoretically any active agent may be used in combination with either the active agent protein or the carrier protein, both the protein and the active agent must be soluble in the same solution or be modified to be soluble in the same solution. One of skill in the art can readily determine the solubility of agents and determine whether they can be used in combination with an active agent protein or a carrier protein as well as whether active agents can be used together.

By “active agent” is intended an agent for delivery to a patient in need thereof. The active agent may find use in the treatment, diagnosis and/or management of a disease state. Such agents include but are not limited to small molecule pharmaceuticals, therapeutic and diagnostic proteins, antibodies, DNA and RNA sequences, imaging agents, and other active pharmaceutical ingredients. Active agents include the active agent proteins listed above. Active agents also include, without limitation, analgesics, anti-inflammatory agents (including NSAIDs), anticancer agents, antimetabolites, anthelmintics, anti-arrhythmic agents, antibiotics, anticoagulants, antidepressants, antidiabetic agents, antiepileptics, antihistamines, antihypertensive agents, antimuscarinic agents, antimycobacterial agents, antineoplastic agents, immunosuppressants, antithyroid agents, antiviral agents, anxiolytic sedatives (hypnotics and neuroleptics), astringents, beta-adrenoceptor blocking agents, blood products and substitutes, cardiac inotropic agents, contrast media, corticosteroids, cough suppressants (expectorants and mucolytics), diagnostic agents, diagnostic imaging agents, diuretics, dopaminergics (antiparkinsonian agents), haemostatics, immunological agents, therapeutic proteins, enzymes, lipid regulating agents, muscle relaxants, parasympathomimetics, parathyroid calcitonin and biphosphonates, prostaglandins, radio-pharmaceuticals, sex hormones (including steroids), anti-allergic agents, stimulants and anoretics, sympathomimetics, thyroid agents, vasodilators, xanthines, and antiviral agents.

Anticancer agents include, without limitation, alkylating agents, antimetabolites, natural products, hormones, topoisomerase I inhibitors, topoisomerase II inhibitors, RNA/DNA antimetabolites, DNA antimetabolites, antimitotic agents and antagonists, and miscellaneous agents, such as radiosensitizers. Examples of alkylating agents include, without limitation, alkylating agents having the bis-(2-chloroethyl)-amine group such as chlormethine, chlorambucile, melphalan, uramustine, mannomustine, extramustinephoshate, mechlore-thaminoxide, cyclophosphamide, ifosfamide, and trifosfamide; alkylating agents having a substituted aziridine group such as tretamine, thiotepa, triaziquone, and mitomycine; alkylating agents of the alkyl sulfonate type, such as busulfan, piposulfan, and piposulfam; alkylating N-alkyl-N-nitrosourea derivatives, such as carnustine, lomustine, semustine, or streptozotocine; and alkylating agents of the mitobronitole, dacarbazine, and procarbazine type. See, for example U.S. Pat. No. 5,399,363. Antimitotic agents include allocolchicine, halichondrin B, colchicine, dolastatin, maytansine, rhizoxin, taxol and taxol derivatices, paclitaxel, vinblastine sulfate, vincristine sulfate, and the like. Topoisomerase I inhibitors include camptothecin, aminocamptothecin, camptothecin derivatives, morpholinodoxorubicin, and the like. Topoisomerase II inhibitors include doxorubicin, amonafide, m-AMSA, anthrapyrazole, pyrazoloacridine, daunorubicin, deoxydoxorubicin, mitoxantrone, menogaril, N,N-dibenzyl daunomycin, oxanthrazole, rubidazone, and the like. Other anticancer agents can include immunosuppressive drugs, such as cyclosporine, azathioprine, sulfasalazine, methoxsalen, and thalidomide.

Antimetabolites include, without limitation, folic acid analogs, such as methotrexate; pyrimidine analogs such as fluorouracil, floxuridine, tegafur, cytarabine, idoxuridine, and flucytosine; and purine derivatives such as mercaptopurine, thioguanine, azathioprine, tiamiprine, vidarabine, pentostatin, and puromycine. Antibiotics also include gentamicin, kanamycin, neomycin, netilmicin, streptomycin, tobramycin, paromomycin, geldanamycin, herbimycin, loracarbef, ertapenem, doripenem, imipenem, cilastatin, meropenem, cefadroxil, cefazolin, cefalotin, cefalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefdinir, cefepime, teicoplanin, vancomycin, azithromycin, clarithromycin, cirithromycin, erythromycin, roxithromycin, troleandomycin, telithromycin, spectinomycin, aztreonam, amoxicillin, ampicillin, azlocillin, carbenicillin, cloxacillin, dicloxacillin, flucloxacillin, mezlocillin, meticillin, nafcillin, oxacillin, penicillin, piperacillin, ticarcillin, bacitracin, colistin, polymyxin B, ciprofloxacin, enoxacin, gatifloxacin, levofloxacin, lomeflxacin, moxifloxacin, norfloxacin, ofloxacin, trovafloxacin, mafenide, prontosil, sulfacetamide, sulfamethizole, sulfanilamide, sulfasalazine, sulfisoxazole, trimethoprim, trimethoprim-sulfamethoxazole, demeclocycline, doxycycline, minocycline, oxytetracycline, tetracycline, arsphenamine, chloramphenicol, clindamycin, lincomycin, ethambutol, fusfomycin, fusidic acid, furazolidone, isoniazid, linezoilid, metronidazole, mupirocin, nitrofurantoin, platensimycin, pyrazinamide, quinupristin, dalfopristin, rifampin, rifampicin, timidazole, etc.

Therapeutic proteins include enzymes, blood factors, blood clotting factors, insulin, erythropoietin, interferons, including interferon-α, interferon-β, protein C, hirudin, granulocyte-macrophage colony-stimulating factor, somatropin, epidermal growth factor, albumin, hemoglobin, lactoferrin, angiotensin-converting enzyme, glucocerebrosidase, human growth hormone, VEGF, antibodies, monoclonal antibodies, including remicade, rituxan, herceptin, bexxar, zevalin, and the like. Proteins also include antigenic proteins or peptides.

As indicated, the active agent may include a polynucleotide. The polynucleotide may be provided as an antisense agent or interfering RNA molecule such as an RNAi or siRNA molecule to disrupt or inhibit expression of an encoded protein. siRNA includes small pieces of double-stranded RNA molecules that bind to and neutralize specific messenger RNA (mRNA) and prevent the cell from translating that particular message into a protein. Alternatively, the polynucleotide may comprise a sequence encoding a peptide or protein of interest such as a therapeutic protein or antigenic protein or peptide. Accordingly, the polynucleotide may be any nucleic acid including but not limited to RNA and DNA. The polynucleotides may be of any size or sequence and may be single- or double-stranded. Methods for synthesis of RNA or DNA sequences are known in the art. See, for example, Ausubel et al. (1999) Current Protocols in Molecular Biology (John Wiley & Sons, Inc., NY); Sambrook et al. (1989) Molecular Cloning: A Laboratory Manual (2nd ed.) (Cold Spring Harbor Laboratory Press, Plainview, N.Y.); herein incorporated by reference.

Examples of natural products include vinca alkaloids, such as vinblastine and vincristine; epipodophylotoxins, such as etoposide and teniposide; antibiotics, such as adriamycine, daunomycine, doctinomycin, daunorubicin, doxorubicin, mithramycin, bleomycin, and mitomycin; enzymes, such as L-asparaginase; biological response modifiers, such as alpha-interferon; camptothecin; taxol; and retinoids, such as retinoic acid.

Other agents include, without limitation, MR imaging agents, contrast agents, gadolinium chelates, gadolinium-based contrast agents, radiosensitizers, such as, for example, 1,2,4-benzotriazin-3-amine 1,4-dioxide (SR 4889) and 1,2,4-benzotriazine-7-amine 1,4-dioxide (WIN 59075); platinum coordination complexes such as cisplatin and carboplatin; anthracenediones, such as mitoxantrone; substituted ureas, such as hydroxyurea; and adrenocortical suppressants, such as mitotane and aminoglutethimide.

Where the protein micro and/or nanoparticle includes at least one additional active agent, it is recognized that a single agent or a combination of agents may be contained within the same nanoparticle. Thus, in some instances, the pharmaceutical organic particles of the invention are a homogeneous mix of nanoparticles. That is, a mixture of nanoparticles containing the same cargo or agent(s). Alternatively, a composition of pharmaceutical organic particles of the invention may comprise a heterogeneous mixture of nanoparticles. That is nanoparticles containing different cargo or agents may be mixed and administered to a patient in need thereof.

It is recognized that the carrier can be selected to target the protein micro and/or nanoparticles to a particular cell type or tissue. The use of albumin as a carrier takes advantage of the albumin receptor-mediated transport across endothelial cell walls of tumor neovasculature. In the same manner, targeting may be accomplished by the use of targeting ligands. The attachment of such targeting ligands would depend upon the carrier and sites of attachments chosen that do no disrupt secondary structure. Methods for linking a targeting molecule to a ligand, binding module, or to a non-binding domain will vary according to the reactive groups present on each carrier. Protocols for linking using reactive groups and molecules are known to one of skill in the art. See, e.g., Goldman et al. (1997) Cancer Res. 57: 1447-1451; Cheng (1996) Hum. Gene Therapy 7: 275-282; Neri et al. (1997) Nat. Biotechnol. 19: 958-961; Nabel (1997) Current Protocols in Human Genetics, vol. on CD-ROM (John Wiley & Sons, New York); Park et al. (1997) Adv. Pharmacol. 40: 399-435; Pasqualini et al. (1997) Nat. Biotechnol. 15: 542-546; Bauminger & Wilchek (1980) Meth. Enzymol. 70: 151-159; U.S. Pat. Nos. 6,280,760 and 6,071,890; and European Patent Nos. 0 439 095 and 0 712 621. The protein micro and/or nanoparticles described herein are generally useful for treatment and/or detection of diseases, including, for example, proliferative diseases such as solid tumors and B-cell related cancers, diseases having an autoimmune/inflammatory component, cardiovascular diseases, diabetes, and the like. As used herein, “treatment” is an approach for obtaining beneficial or desired clinical results. For purposes of this invention, beneficial or desired clinical results include, but are not limited to, any one or more of: alleviation of one or more symptoms, diminishment of extent of disease, stabilized (i.e., not worsening) state of disease, preventing or delaying spread (e.g., metastasis) of disease, preventing or delaying occurrence or recurrence of disease, delay or slowing of disease progression, amelioration of the disease state, and remission (whether partial or total). Also encompassed by “treatment” is a reduction of pathological consequence of a disease. The methods of the invention contemplate any one or more of these aspects of treatment.

Solid tumors that can be treated and/or detected using the protein micro and/or nanoparticles of the present invention include, but are not limited to, breast cancer (which may be HER2 positive or HER2 negative), ovarian cancer, cervical cancer, colorectal cancer, prostate cancer, renal cancer (including, for example, renal cell carcinomas), cancer of the bladder, cancer of the liver (including, for example, hepatocellular carcinomas), gastrointestinal cancer, pancreatic cancer, lung cancer (for example, non-small cell lung cancer of the squamous cell carcinoma, adenocarcinoma, and large cell carcinoma types, and small cell lung cancer), nasopharyngeal cancer, thyroid cancer (for example, thyroid papillary carcinoma), cancers of the head and neck, neuroblastomas, and skin cancers such as melanoma, and sarcomas (including, for example, osteosarcomas and Ewing's sarcomas).

Examples of B-cell related cancers that can be treated and/or detected using the protein micro and/or nanoparticles of the present invention include, but are not limited to, non-Hodgkin's lymphoma, chronic lymphocytic leukemia, multiple myeloma, B cell lymphoma, high-grade B cell lymphoma, intermediate-grade B cell lymphoma, low-grade B cell lymphoma, B cell acute lympohoblastic leukemia, myeloblastic leukemia, Hodgkin's disease, plasmacytoma, follicular lymphoma, follicular small cleaved lymphoma, follicular large cell lymphoma, follicular mixed small cleaved lymphoma, diffuse small cleaved cell lymphoma, diffuse small lymphocytic lymphoma, prolymphocytic leukemia, lymphoplasmacytic lymphoma, marginal zone lymphoma, mucosal associated lymphoid tissue lymphoma, monocytoid B cell lymphoma, splenic lymphoma, hairy cell leukemia, diffuse large cell lymphoma, mediastinal large B cell lymphoma, lymphomatoid granulomatosis, intravascular lymphomatosis, diffuse mixed cell lymphoma, diffuse large cell lymphoma, immunoblastic lymphoma, Burkitt's lymphoma, AIDS-related lymphoma, and mantle cell lymphoma.

The protein micro and/or nanoparticles of the present invention can be designed for treatment of diseases comprising an autoimmune and/or inflammatory component. Such diseases include but are not limited to autoimmune and inflammatory diseases such as systemic lupus erythematosus (SLE), discoid lupus, lupus nephritis, sarcoidosis, inflammatory arthritis, including, but not limited to, juvenile arthritis, rheumatoid arthritis, psoriatic arthritis, Reiter's syndrome, ankylosing spondylitis, and gouty arthritis, rejection of an organ or tissue transplant, hyperacute, acute, or chronic rejection and/or graft versus host disease, multiple sclerosis, hyper IgE syndrome, polyarteritis nodosa, primary biliary cirrhosis, inflammatory bowel disease, Crohn's disease, celiac's disease (gluten-sensitive enteropathy), autoimmune hepatitis, pernicious anemia, autoimmune hemolytic anemia, psoriasis, scleroderma, myasthenia gravis, autoimmune thrombocytopenic purpura, autoimmune thyroiditis, Grave's disease, Hasimoto's thyroiditis, immune complex disease, chronic fatigue immune dysfunction syndrome (CFIDS), polymyositis and dermatomyositis, cryoglobulinemia, thrombolysis, cardiomyopathy, pemphigus vulgaris, pulmonary interstitial fibrosis, sarcoidosis, Type I and Type II diabetes mellitus, type 1, 2, 3, and 4 delayed-type hypersensitivity, allergy or allergic disorders, unwanted/unintended immune responses to therapeutic proteins, asthma, Churg-Strauss syndrome (allergic granulomatosis), atopic dermatitis, allergic and irritant contact dermatitis, urtecaria, IgE-mediated allergy, atherosclerosis, vasculitis, idiopathic inflammatory myopathies, hemolytic disease, Alzheimer's disease, chronic inflammatory demyelinating polyneuropathy, and the like.

Cardiovascular diseases that can beneficially be treated using the protein micro and/or nanoparticles of the invention include, but are not limited to, atherosclerosis, coronary artery disease, carotid artery disease, peripheral artery disease (PAD), hypercholesterolemia/hyperlipidemia, stroke, high blood pressure/hypertension, restenosis, stenosis, and heart attack (coronary thrombosis, myocardial infarction).

The protein micro and/or nanoparticles of the invention are formulated such that an effective amount of the active agent can be administered to a subject in need thereof. The term “effective amount” as used herein refers to an amount of the active agent sufficient to treat a specified disorder, condition or disease such as to ameliorate, palliate, lessen, and/or delay one or more of its symptoms. In reference to cancers or other unwanted cell proliferation, an effective amount comprises an amount sufficient to cause a tumor to shrink and/or to decrease the growth rate of the tumor (such as to suppress tumor growth) or to prevent or delay other unwanted cell proliferation. In some embodiments, an effective amount is an amount sufficient to delay development. In some embodiments, an effective amount is an amount sufficient to prevent or delay occurrence and/or recurrence. An effective amount can be administered in one or more administrations. In the case of cancer, the effective amount of the drug or composition may: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; and/or (vii) relieve to some extent one or more of the symptoms associated with the cancer.

In one embodiment the present invention is broadly directed to pharmaceutical organic micro and nanoparticles for cancer therapy. In general, the pharmaceutical organic micro and nanoparticles of the present invention use natural proteins to provide systemically friendly cancer therapies while shielding and solubilizing the active agent(s). The pharmaceutical organic micro and nanoparticles develop an effective delivery system for use in nanomedicine and are fabricated using PRINT™ technology (Particle Replication in Non-wetting Templates) (Liquidia Technologies, Inc., Research Triangle Park, N.C.), which takes nanomedicine to the next level by allowing predetermined engineering of the parameters of an ideal nanoparticle delivery vehicle. PRINT™ technology utilizes liquid polymers or Fluorocur™ (Liquidia Technologies, Inc., Research Triangle Park, N.C.) to replicate micro or nano sized structures on a master template. The polymers utilized in PRINT™ molds are liquid at room temperature and can be photo-chemically cross-linked into elastomeric solids that enable high resolution replication of micro or nano sized structures. The liquid polymer is then cured while in contact with the master, thereby forming a replica image of the structures on the master. Upon removal of the cured liquid polymer from the master template, the cured liquid polymer forms a patterned template that includes cavities or recess replicas of the micro or nano-sized features of the master template and the micro or nano-sized cavities in the cured liquid polymer can be used for high-resolution micro or nanoparticle fabrication. For more detailed description of the materials used to fabricate the molds of the present invention and methods of molding micro or nanoparticles in the molds see U.S. patent application Ser. Nos. 10/583,570, filed Jun. 19, 2006, and 11/594,023 filed Nov. 7, 2006; and PCT International Patent Application Serial Nos.: PCT/US04/42706, filed Dec. 20, 2004; PCT/US/06/23722, filed Jun. 19, 2006; PCT/US06/34997, filed Sep. 7, 2006; PCT/US06/43305, filed Nov. 7, 2006; and PCT/US07/02476, filed Jan. 29, 2007; each of which is incorporated herein by reference in its entirety. See also, U.S. Provisional Patent Application Ser. Nos. 60/531,531, filed Dec. 19, 2003; 60/583,170, filed Jun. 25, 2004; 60/604,970 filed Aug. 27, 2004; 60/691,607, filed on Jun. 17, 2005; 60/714,961, filed Sep. 7, 2005; 60/762,802, filed Jan. 27, 2006; 60/798,858, filed May 9, 2006; 60/734,228, filed Nov. 7, 2005; 60/757,411, filed Jan. 9, 2006; 60/799,876, filed May 12, 2006; 60/833,736, filed Jul. 27, 2006; and 60/828,719, filed Oct. 9, 2006; each of which is incorporated herein by reference it its entirety.

The pharmaceutical organic particles of the present invention allow for precise control over particle size and particle shape down to the nanometer, particle composition (i.e., organic/inorganic, solid/porous, textured/untextured), particle cargo (i.e., hydrophilic or hydrophobic therapeutic molecules, biologicals, peptides, proteins, oligonucleotides, siRNA, imaging agents such as MR contrast agents, Gd nanoparticles (ethylene coated Gd-oxide), positron emitters, fluorophores, etc), particle physical properties such as modulus (i.e., rigid, flexible, deformable) and particle surface properties (i.e., avidin/biotin complexes, targeting peptides, antibodies, aptamers, cationic/anion charges, stealth PEG chains for steric stabilization). Therefore, the organic particles of the present invention are truly engineered drug therapies. Key therapeutic parameters such as bioavailability, biodistribution, and target-specific cell penetration can be designed into a therapy.

The methods and materials used to fabricate the organic particles of the present invention are delicate and versatile enough to be compatible with a wide variety of biomaterials targeted for advanced understandings and therapies in disease prevention, detection, diagnosis and treatment. To date, substantially non-disperse or monodisperse particles have been fabricated from a wide range of particle matrix materials including biocompatible poly(ethylene glycol) and bioabsorbable poly(D-lactic acid). Truly non-disperse, shape-specific fully bioabsorbable nanoparticles have never been fabricated before. The uniformity of the organic particles has been confirmed using dynamic light scattering (DLS) and scanning electron microscopy. The compatibility of the organic particle fabrication processes with fragile biological cargos has been demonstrated by incorporating proteins, DNA, and anti-cancer agents such as doxorubicin into PEG nanoparticles. For certain applications, the controlled release of cargos from the matrix materials is desired. For example, once the organic particles enter a cell, the carriers can be designed to release their cargo via several different mechanisms including: i) diffusion-controlled release of the cargo by varying the physical properties of the particle matrix (charge and mesh size), ii) release based on triggered degradation, or iii) desired swelling of the particle matrix.

I. Mold Materials

Representative materials useful in fabricating the cavities from which pharmaceutical organic particles of the present invention can be formed include elastomer-based materials. The elastomer-based materials include, but are not limited to, fluorinated elastomer-based materials, solvent resistant elastomer based materials, combinations thereof, and the like. As used herein, the term “solvent resistant” refers to a material, such as an elastomeric material that either does not swell or does not substantially swell nor dissolve or substantially dissolve in common hydrocarbon-based organic solvents, or reagents, or acidic or basic aqueous solutions. Representative fluorinated elastomer-based materials include but are not limited to fluoropolyether and perfluoropolyether (PFPE) based materials. For ease of discussion the remainder of this specification will primarily describe PFPE based materials, however, it should be appreciated that the articles and methods disclosed and enabled herein can be applied to or with other materials.

The materials that are used to fabricate the molds or cavities from which the pharmaceutical organic particles are formed are typically liquid polymers at room temperature and can be made curable by addition of a thermal curable constituent, photo curable constituent, combination thereof, or the like. According to another embodiment, the material for forming the cavities includes one or more of a photo-curable constituent, a thermal-curable constituent, mixtures thereof, and the like. In one embodiment, the cavity material includes a photo-curable constituent and a thermal-curable constituent such that the material can undergo multiple cures. A material having the ability to undergo multiple cures is useful, for example, in forming articles of the present invention. For example, a liquid material having dual cure ability can include a material having a photo-curable and a thermal-curable constituent, two photo-curable constituents that cure at different wavelengths, two thermal-curable constituents that cure at different temperatures, or the like. In some embodiments, photo-curable and thermal-curable constituents can undergo a first cure through, for example, a photocuring process or a thermal curing process such that an article is first cured. Then the first photocured or thermal cured article can be subjected to a second cure to activate the curable component not activated in the first cure. In some embodiments, a first cured article can be adhered to a second cured article of the same material or any material similar thereto that will thermally cure or photocure and bind to the material of the first cured article. By positioning the first cured article and second cured article adjacent one another and subjecting the first and second cured articles to a thermal curing or photocuring process, whichever component that was not activated on the first cure can be cured by a subsequent curing step. Thereafter, either the thermal cure constituents of the first cured article that was left un-activated by the photocuring process or the photocure constituents of the first cured article that were left un-activated by the first thermal curing, will be activated and bind the second article. Thereby, the first and second articles become adhered together. It will be appreciated by one of ordinary skill in the art that the order of curing processes is independent and a thermal-curing could occur first followed by a photocuring, a photocuring could occur first followed by a thermal curing, or the like.

According to yet another embodiment, multiple thermo-curable constituents can be included in the material such that the material can be subjected to multiple independent thermal-cures. For example, the multiple thermo-curable constituents can have different activation temperature ranges such that the material can undergo a first thermal-cure at a first temperature range and a second thermal-cure at a second temperature.

According to one embodiment the PFPE material has a surface energy below about 30 mN/m. According to another embodiment the surface energy of the PFPE is between about 10 mN/m and about 20 mN/m. According to another embodiment, the PFPE has a low surface energy of between about 12 mN/m and about 15 mN/m. In some embodiments, the surface energy is less than about 12 mN/m.

The PFPE is non-toxic, UV transparent, and highly gas permeable; and cures into a tough, durable, highly fluorinated elastomer with excellent release properties and resistance to swelling. The properties of these materials can be tuned over a wide range through the judicious choice of additives, fillers, reactive co-monomers, and functionalization agents. Such properties that are desirable to modify, include, but are not limited to, modulus, tear strength, surface energy, permeability, functionality, mode of cure, solubility and swelling characteristics, and the like. The non-swelling nature and easy release properties of the presently disclosed PFPE materials allows for nanostructures to be fabricated from nearly any material. Further, the presently disclosed subject matter can be expanded to large scale rollers or conveyor belt technology or rapid stamping that allow for the fabrication of nanostructures on an industrial scale.

In other embodiments, the material for forming the molds can include, but is not limited to, a perfluoropolyether material, a fluoroolefin material, an acrylate material, a silicone material, a styrenic material, a fluorinated thermoplastic elastomer (TPE), a triazine fluoropolymer, a perfluorocyclobutyl material, a fluorinated epoxy resin, and a fluorinated monomer or fluorinated oligomer that can be polymerized or crosslinked by a metathesis polymerization reaction.

In some embodiments, the fluoroolefin material is made from monomers which include tetrafluoroethylene, vinylidene fluoride, hexafluoropropylene, 2,2-bis(trifluoromethyl)-4,5-difluoro-1,3-dioxole, a functional fluoroolefin, functional acrylic monomer, and a functional methacrylic monomer. In some embodiments, the silicone material includes a fluoroalkyl functionalized polydimethylsiloxane (PDMS). In some embodiments, the styrenic material includes a fluorinated styrene monomer. In some embodiments, the acrylate material includes a fluorinated acrylate or a fluorinated methacrylate. In some embodiments, the triazine fluoropolymer includes a fluorinated monomer. In some embodiments, the fluorinated monomer or fluorinated oligomer that can be polymerized or crosslinked by a metathesis polymerization reaction includes a functionalized olefin. In some embodiments, the functionalized olefin includes a functionalized cyclic olefin. According to an alternative embodiment, the PFPE material includes a urethane block, such as PFPE urethane tetrafunctional methacrylate materials, can be used as the materials and methods of the present subject matter.

From a property point of view, the exact properties of these materials can be adjusted by adjusting the composition of the ingredients used to make the materials. In particular the modulus can be adjusted from low (approximately 1 MPa) to multiple GPa.

II. Formation of Isolated Micro- and/or Nano Particles

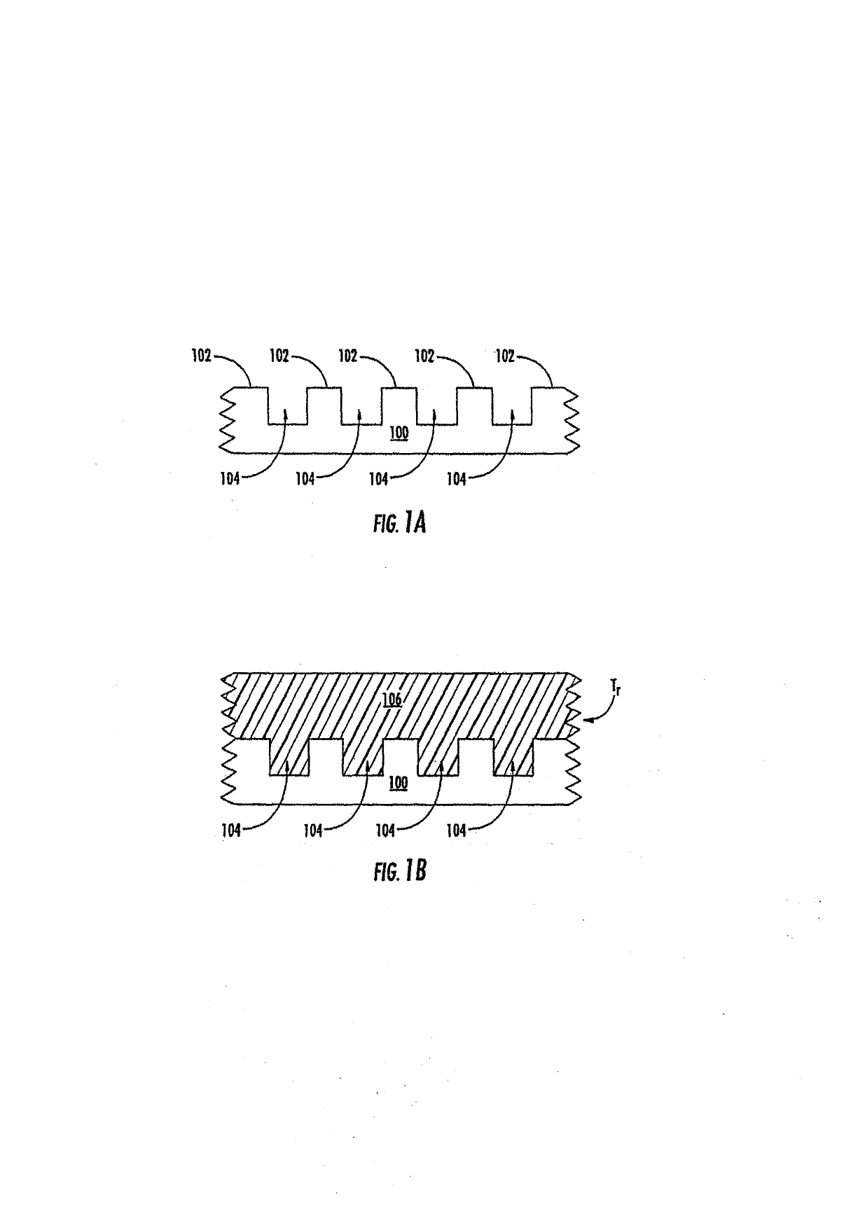

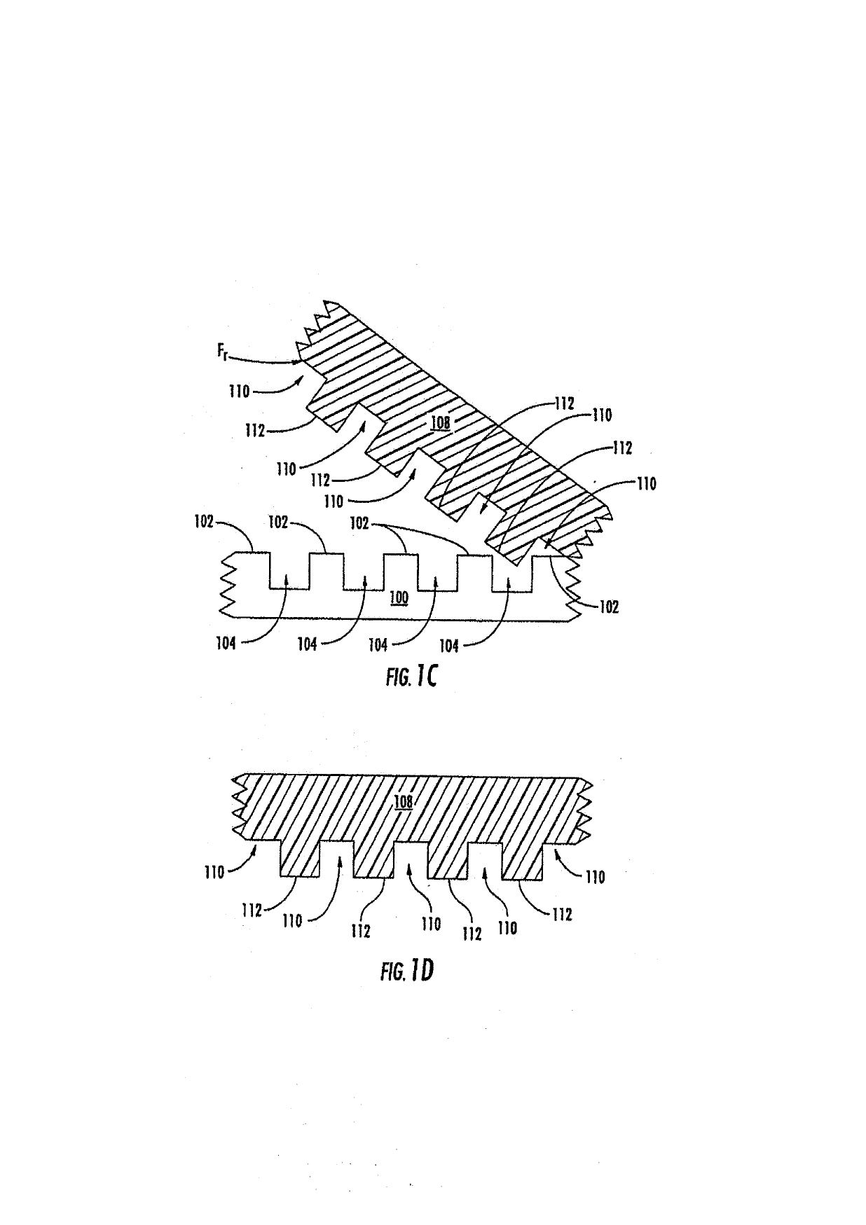

In some embodiments, the present subject matter provides methods, materials, and articles for making pharmaceutical organic micro- and/or nanoparticles. Turning now to FIG. 1A, patterned master 100 is provided. Patterned master 100 includes a plurality of non-recessed surface areas 102 and a plurality of recesses or cavities 104. In some embodiments, patterned master 100 includes an etched substrate, such as a silicon wafer, which is etched or otherwise fabricated into a predetermined pattern.

Referring now to FIG. 1B, a liquid material 106, for example, a liquid fluoropolymer composition disclosed herein, such as a PFPE-based precursor, is then introduced onto patterned master 100. Liquid material 106 is treated by treating process Tr, for example exposure to UV light, actinic radiation, thermal exposure, or the like, thereby forming a treated liquid material 108 in the desired pattern.

Referring now to FIGS. 1C and 1D, a force Fr is applied to treated liquid material 108 to remove it from patterned master 100. As shown in FIGS. 1C and 1D, treated liquid material 108 includes a plurality of cavities 110, which are mirror images of the plurality of non-recessed surface areas 102 of patterned master 100. Continuing with FIGS. 1C and 1D, treated liquid material 108 includes a plurality of first patterned surface areas 112, which are mirror images of the plurality of cavities 104 of patterned master 100. Accordingly, treated liquid material 108 can be used as a patterned template having cavities for which micro- and nanoparticles can be formed.

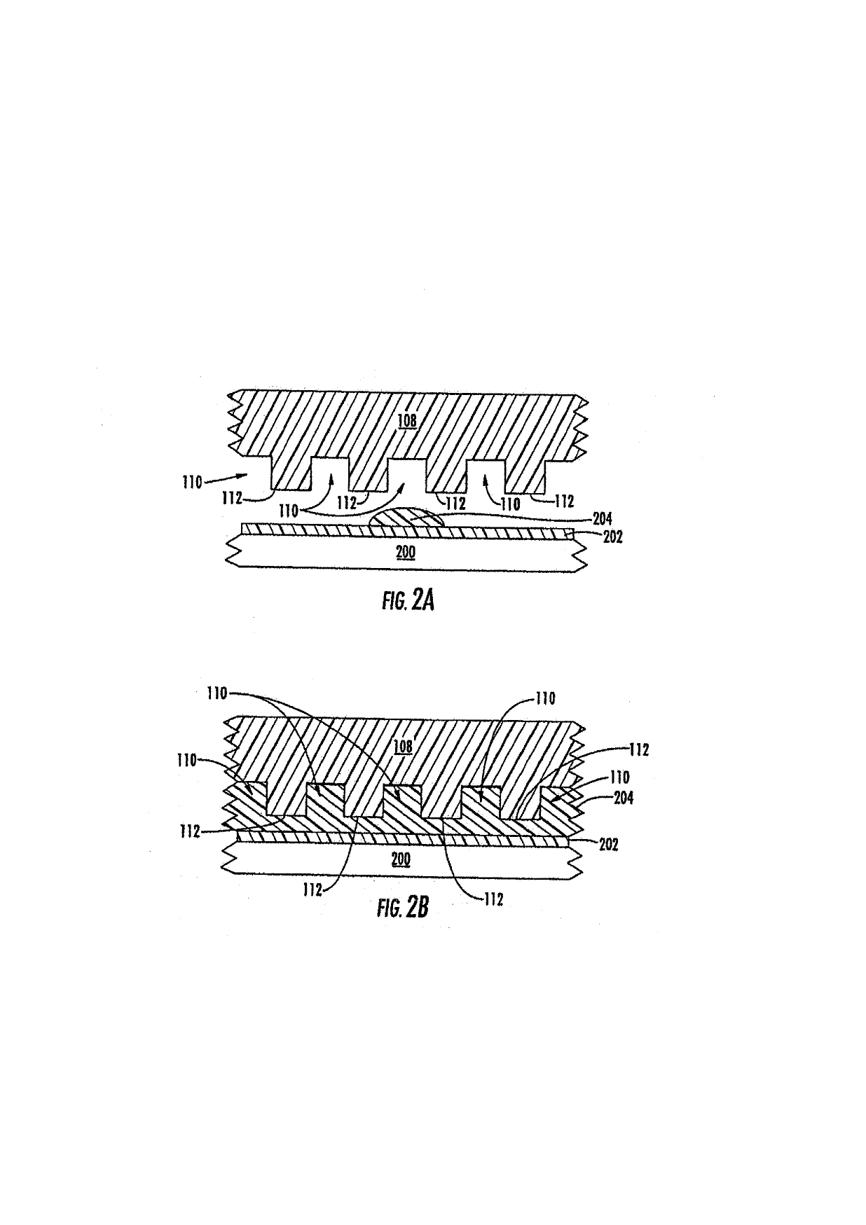

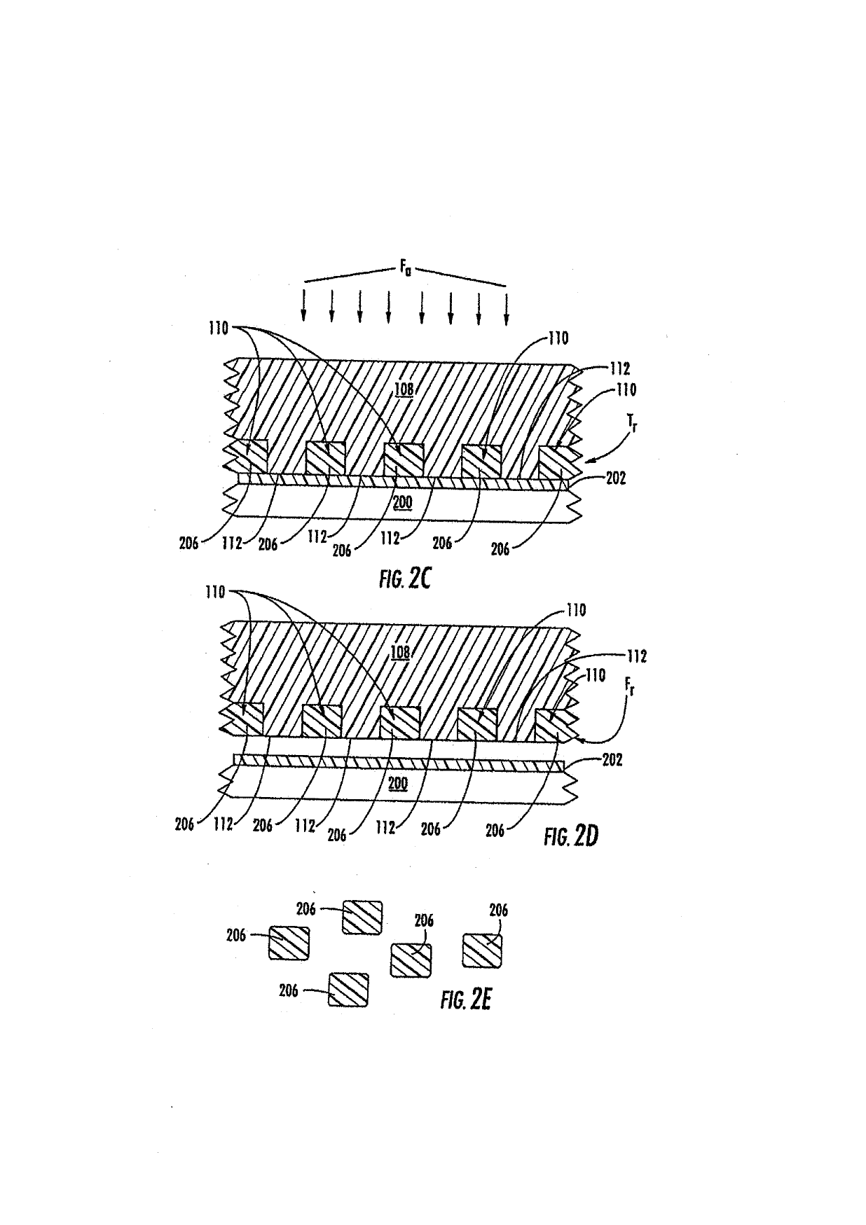

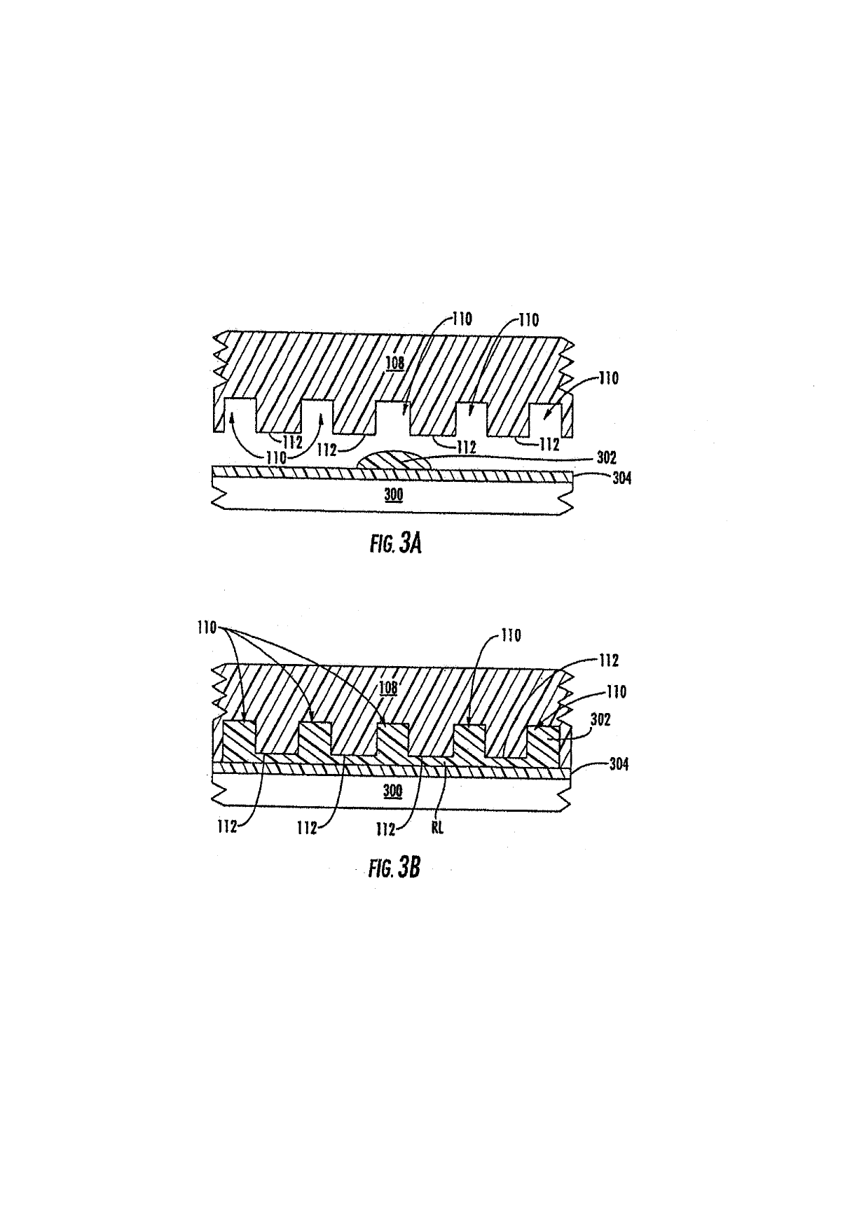

Referring now to FIGS. 2A and 2B, patterned template 108 is then contacted with droplet 204 of a particle precursor material so that droplet 204 fills the plurality of cavities or recessed areas 110 of patterned template 108. Referring now to FIGS. 2C and 2D, a force Fa can be applied to patterned template 108. In some embodiments, as force Fa is applied the force Fa causes droplet 204 to be excluded from all areas except for cavity areas 110. In some embodiments, a vacuum or other force can be applied to remove trapped gases from cavities 110 prior to introducing particle precursor material 204 such that particle precursor material 204 enters and/or completely fills cavities 110. In other embodiments, excess droplet material 204 can be used such that the material in the recessed cavities is interconnected. In yet other embodiments, the patterned template can be essentially free of non-wetting or low wetting material 202 such that when droplet 204 is contacted with the patterned template droplet material 204 wets the surface and a scum layer is formed that can interconnect the material in the recessed areas.

In other embodiments, patterned template 108 is contacted with droplet 204. The liquid material including droplet 204 then enters cavity areas 110 of patterned template 108. According to some embodiments, mechanical or physical manipulation of droplet 204 and patterned template 108 is provided to facilitate droplet 204 in substantially filling and conforming to cavity areas 110. Such mechanical and/or physical manipulation can include, but is not limited to, vibration, rotation, centrifugation, pressure differences, a vacuum environment, combinations thereof, or the like. Particles 206 are formed in the cavity areas 110 of patterned template 108. In some embodiments, the mechanical force is applied by contacting one of a doctor blade and a brush with the one or more particles. In some embodiments, the mechanical force is applied by ultrasonics, megasonics, electrostatics, or magnetics. In some embodiments, the force applied to remove trapped gas from cavities 110 and/or assist filling of cavities 110 with particle precursor material can be selected from the group of vibration, rotation, agitation, sonication, vacuum, combinations thereof, or the like.

Continuing with FIGS. 2C and 2D, the particle precursor material filling cavity areas 110 is then treated by a treating process Tr, e.g., photocured, UV-light treated, actinic radiation treated, evaporation, temperature change, centrifuged, phase change, chemical, physical, combinations thereof, or the like, to form a plurality of micro- and/or nanoparticles 206. In some embodiments, a material, including but not limited to a polymer, an organic compound, or an inorganic compound, can be dissolved in a solvent, patterned using patterned template 108, and the solvent can be released. Once the material filling cavities 110 is treated or hardened, patterned template 108 is removed from substrate 200. Micro- and/or nanoparticles 206 are confined to cavity areas 110 of patterned template 108. In some embodiments, micro- and/or nanoparticles 206 can be retained on substrate 200 in defined regions once patterned template 108 is removed.

Referring now to FIGS. 2D and 2E, pharmaceutical organic micro- and/or nanoparticles 206 can be removed from patterned template 108 to provide freestanding particles by a variety of methods, which include but are not limited to: applying patterned template 108 to a surface that has an affinity for the particles 206; deforming patterned template 108, or using other mechanical methods, including sonication or brushing, in such a manner that the particles 206 are naturally released from patterned template 108; swelling patterned template 108 reversibly with supercritical carbon dioxide or another solvent that will extrude the particles 206; washing patterned template 108 with a solvent that has an affinity for the particles 206 and will wash them out of patterned template 108; applying patterned template 108 to a liquid that when hardened physically entraps particles 206; applying patterned template 108 to a material that when hardened has a chemical and/or physical interaction with particles 206; combinations thereof; and the like.

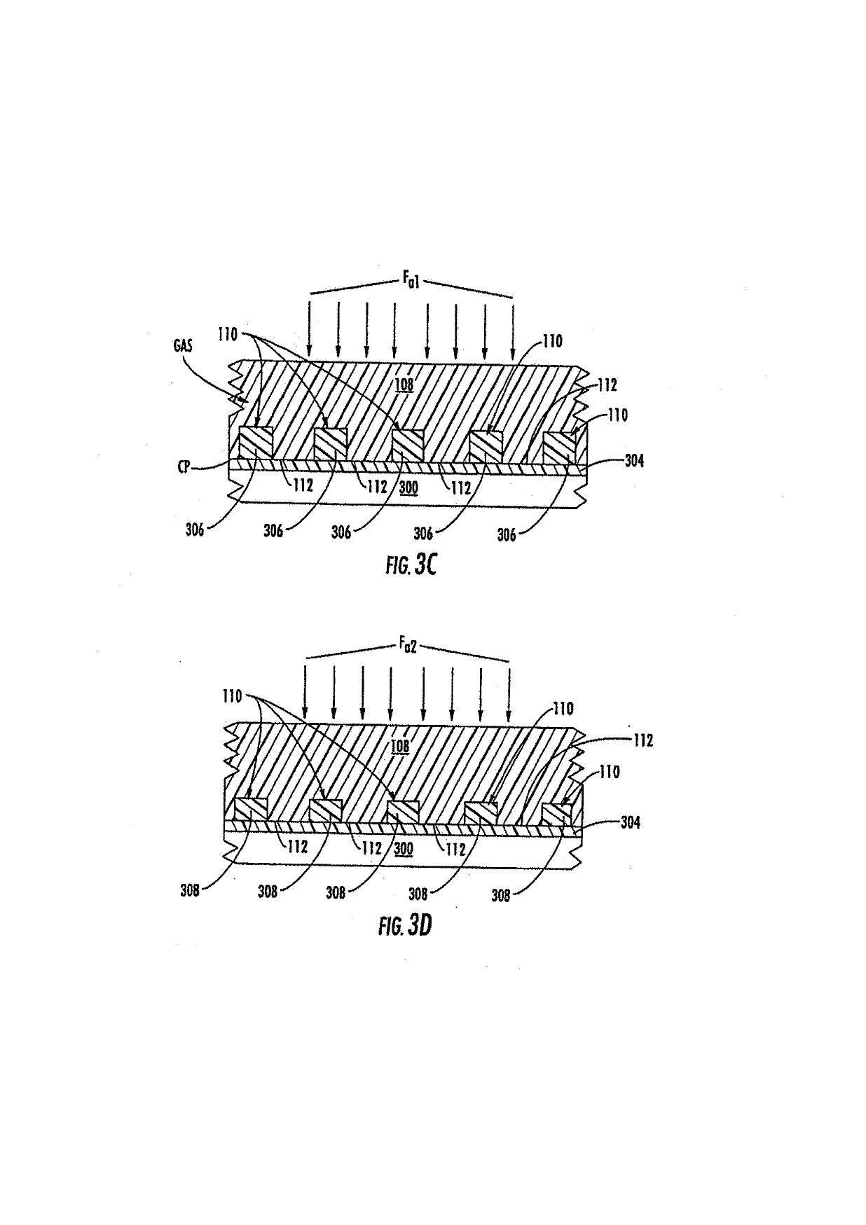

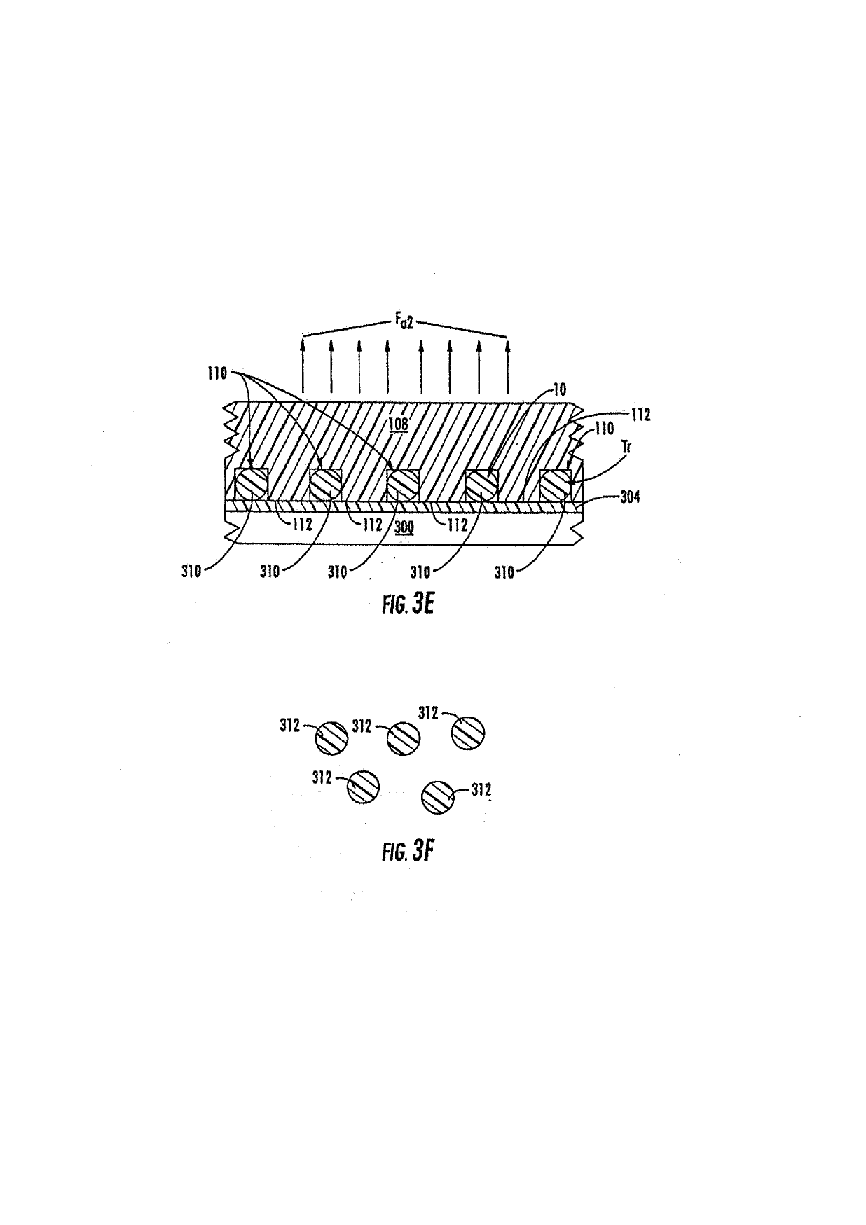

Referring now to FIGS. 3A through 3F, a “liquid reduction” process is provided for forming particles in the cavities of the patterned template, including but not limited to spherical and non-spherical, regular and non-regular micro- and nanoparticles. For example, a “cube-shaped” template cavity can allow for spherical particles to be made, whereas a “Block arrow-shaped” template cavity can allow for “lolli-pop” shaped particles or objects to be made wherein the introduction of a gas allows surface tension forces to reshape the resident liquid prior to treating it. While not wishing to be bound by any particular theory, the non-wetting characteristics that can be provided in some embodiments of the presently disclosed patterned template and/or treated or coated substrate allows for the generation of rounded, e.g., spherical or substantially spherical particles.

Referring now to FIG. 3A, droplet 302 of a liquid material is disposed on substrate 300, which in some embodiments is coated or treated with a non-wetting material 304. A patterned template 108, which includes a plurality of cavity areas 110 and patterned surface areas 112, also is provided.

Referring now to FIG. 3B, patterned template 108 is contacted with droplet 302. The liquid material including droplet 302 then enters cavity areas 110 of patterned template 108. In some embodiments, a residual, or “scum,” layer RL of the liquid material including droplet 302 remains between the patterned template 108 and substrate 300.

Referring now to FIG. 3C, a first force Fa1 is applied to patterned template 108. A contact point CP is formed between the patterned template 108 and the substrate and displacing residual layer RL. Particles 306 are formed in the cavity areas 110 of patterned template 108.

Referring now to FIG. 3D, a second force Fa2, wherein the force applied by Fa2 is greater than the force applied by Fa1, is then applied to patterned template 108, thereby forming smaller liquid particles 308 inside recessed areas 112 and forcing a portion of the liquid material including droplet 302 out of recessed areas 112.

Referring now to FIG. 3E, the second force Fa2 is released, thereby returning the contact pressure to the original contact pressure applied by first force Fa1. In some embodiments, patterned template 108 includes a gas permeable material, which allows a portion of space with recessed areas 112 to be filled with a gas, such as nitrogen, thereby forming a plurality of liquid spherical droplets 310. Once this liquid reduction is achieved, the plurality of liquid spherical droplets 310 are treated by a treating process Tr. Referring now to FIG. 3F, treated liquid spherical droplets 310 are released from patterned template 108 to provide a plurality of freestanding spherical particles 312.

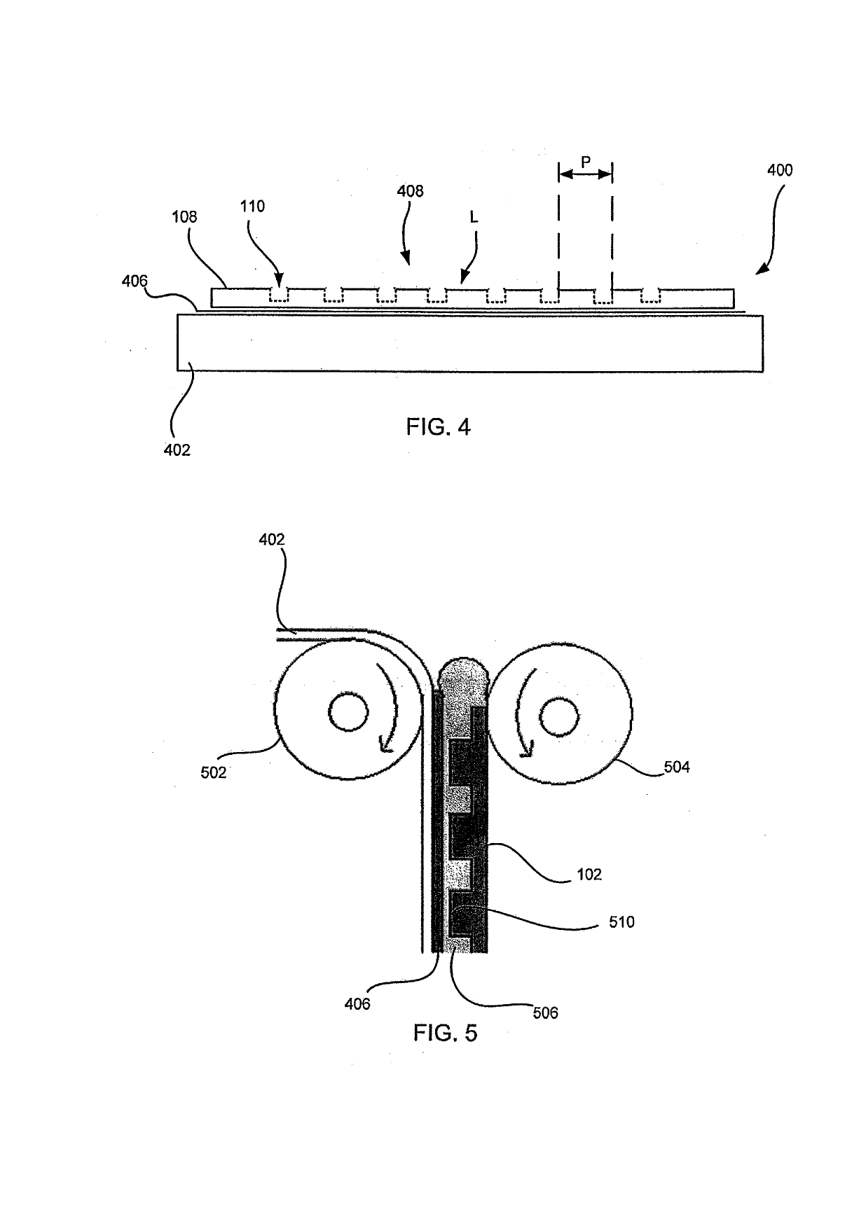

In some embodiments, as shown in FIG. 4, particles 206 are fabricated from laminate molds, such as laminate mold 400 that includes a backing layer 402 affixed to a patterned mold layer 108 by a tie-layer 406. In certain embodiments, tie-layer 406 is used to bond patterned layer 108 to backing layer 402. According to some embodiments, patterned layer 108 includes a patterned surface 408. Patterned layer 108 can be made from the materials disclosed herein, the references incorporated herein by reference, and combinations thereof. According to some embodiments, patterned layer 108 includes a patterned surface 408. Patterned layer 108 can be made from the materials disclosed herein, and combinations thereof. Patterns on patterned surface 408 can include cavities 110 and land area L that extends between cavities 110. Patterns on patterned surface 408 can also include a pitch, such as pitch P, which is generally the distance from a first edge of one cavity to a first edge of an adjacent cavity including land area L between the adjacent cavities.

According to some embodiments, as shown in FIG. 5, laminate mold 400 is fabricated according to the methods and materials disclosed in U.S. patent application Ser. No. 11/633,763, filed on Dec. 4, 2006, which is incorporated herein by reference in its entirety. Referring to FIG. 5, polymer sheet backing 402 is pinched between two rollers 502, 504 adjacent a patterned master 102. As polymer sheet 402 and patterned master 102 are processed through rollers 502, 504, a curable liquid polymer 506 such as PFPE is introduced between an interface of polymer sheet 402 and patterned master 102. Pressure exerted by rollers 502 and 504 force liquid polymer 506 into surface features 510 of patterned master 102 such that surface features 510 are replicated on liquid polymer 506 layer. Next, a curing step cures liquid polymer 506 such that patterned structures 510 are affixed in the cured liquid polymer 506. In some embodiments, a tie layer 508 is configured between polymer backing 402 and cured liquid polymer layer 506, as described in the above referenced patent application.

Accordingly, cavities 110 for fabricating particles according to the methods and materials of the present invention can be fabricated in the cured liquid polymer layer 506 of laminate mold 400.

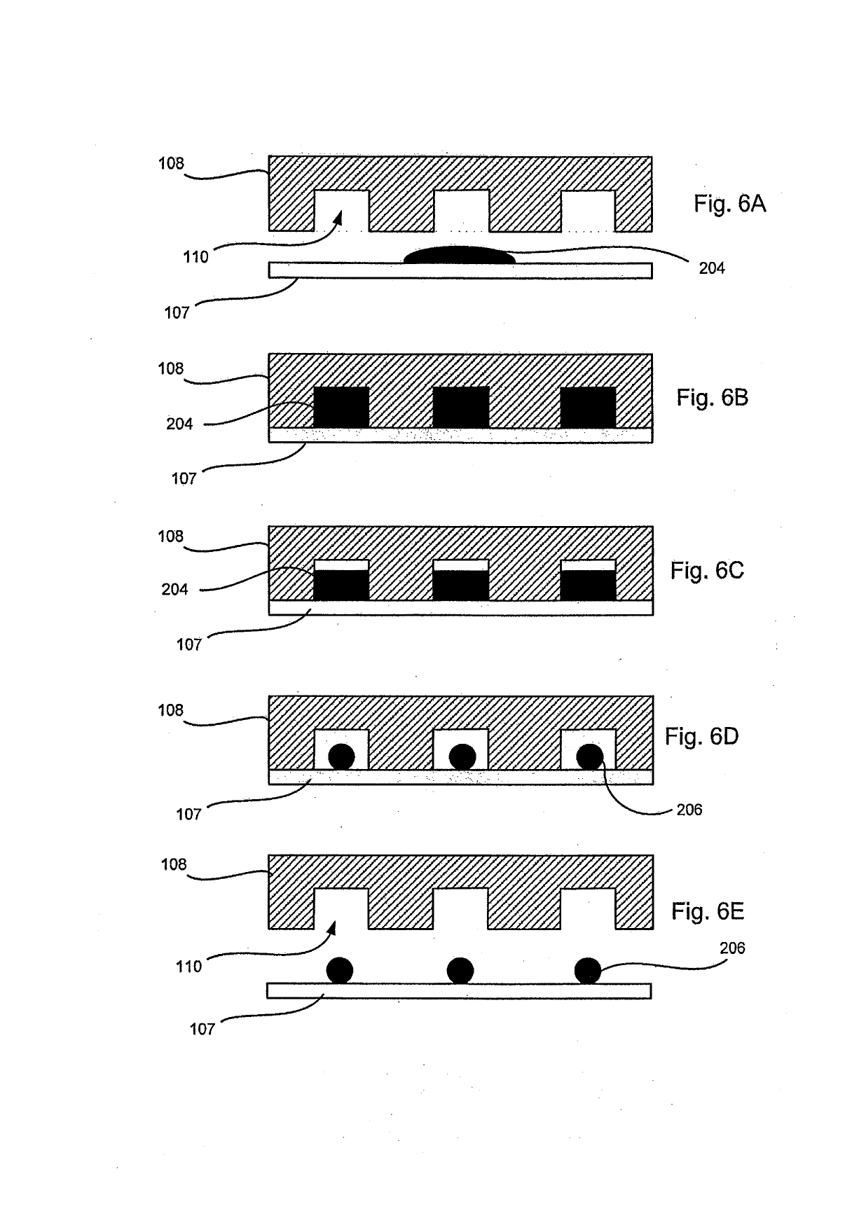

Referring now to FIGS. 6A-6E, an embodiment of the present subject matter includes a process for forming pharmaceutical organic particles through evaporation. In one embodiment, the process produces a particle having a shape that does not necessarily conform to the shape of the cavity. The shape can include virtually any three dimensional shape. According to some embodiments, the particle forms a spherical or non-spherical and regular or non-regular shaped micro- and nanoparticle. According to one embodiment, a spherical or substantially spherical particle 206 can be formed by using a patterned template 108 and/or substrate 107 of a non-wetting material or treating the surfaces of the patterned template with a non-wetting agent such that the material from which the particle will be formed does not wet the surfaces of the cavities of the patterned template. Because the material from which the particle will be formed cannot wet the surfaces of the patterned template 108 and/or cavities 110 particle material 204 has a greater affinity for itself than the surfaces of the cavities and thereby forms a rounded, curved, or substantially spherical shape.

Examples of an evaporative process that can be used with the present embodiments include forming patterned template 108 from a gas permeable material, which allows volatile components of the material to become the particles to pass through the template, thereby reducing the volume of the material to become the particles in the cavities. According to another embodiment, an evaporative process suitable for use with the presently disclosed subject matter includes providing a portion of the recessed cavities 110 filled with a gas, such as nitrogen, which thereby increases the evaporation rate of the material to become the particles. According to further embodiments, after the cavities are filled with material to become the particles, a space can be left between the patterned template and substrate such that evaporation is enhanced. In yet another embodiment, the combination of the patterned template, substrate, and material to become the particle can be heated or otherwise treated to enhance evaporation of the material to become the particle. In other embodiments, the filled mold is lyophilized to produce the particle.

According to some embodiments, the pharmaceutical organic particles described herein are formed in an open mold. Generally, open molding allows for evaporation or reduction of substances introduced into the cavities because a surface of the cavities are left open to an environment. Open molding can reduce the number of steps and sequences of events required during molding of particles and additionally can improve the evaporation rate of solvent from the particle precursor material, thereby, increasing the efficiency and rate of particle production. Further descriptions of open molding techniques and devices can be found in the patent applications incorporated herein by reference.

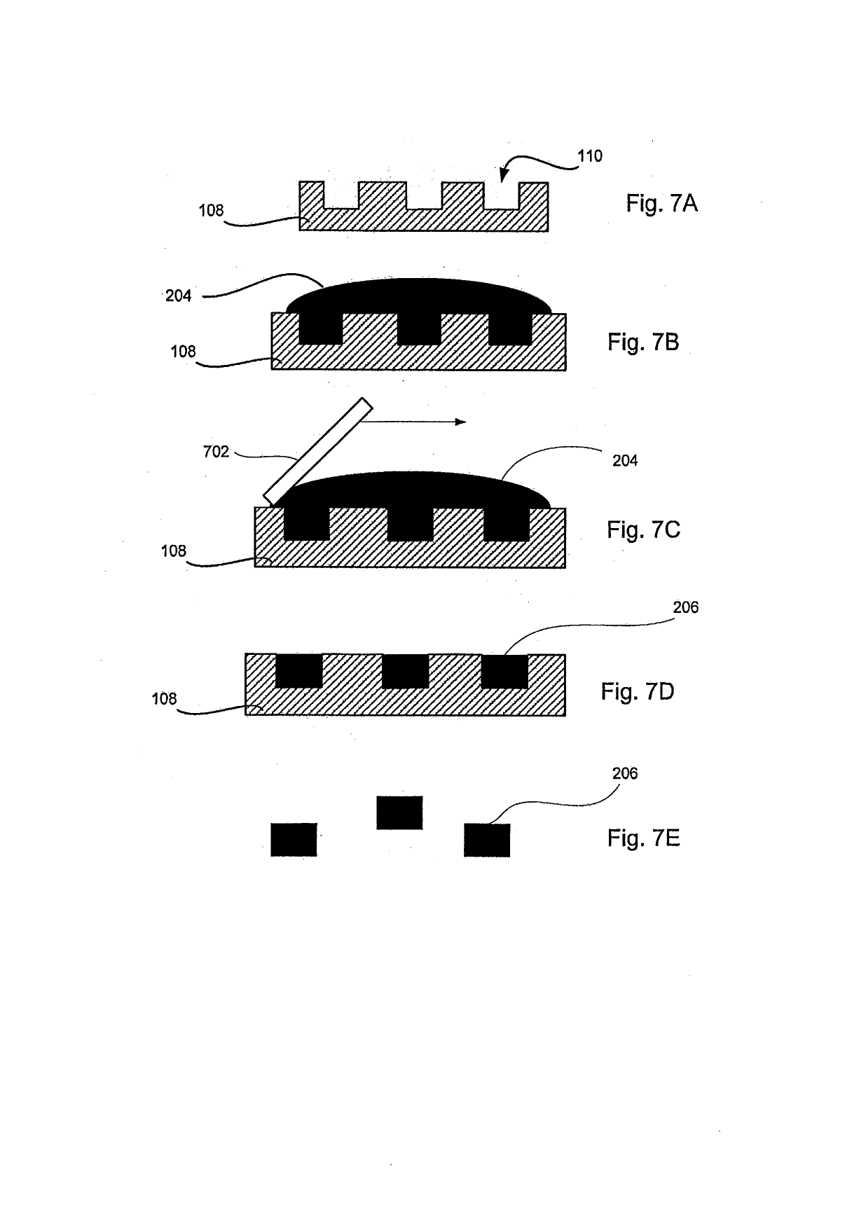

Referring to FIG. 7, surface or template 108 includes cavities 110 formed therein. A substance 204 for forming pharmaceutical organic particles of the present invention can be, but is not limited to a liquid, a powder, a paste, a gel, a liquified solid, combinations thereof, and the like, is then deposited on surface 108. The substance 204 is introduced into cavities 110 of surface 108 and excess substance remaining on surface of patterned template 108 is removed by an active process or by a passive material property process 702. According to some embodiments of active process removal, excess substance 204 can be removed from the surface by, doctor blading 702, applying pressure with a substrate, capillary forces, electrostatics, magnetic forces, gravitational forces, air pressure, vacuum, lyophilizing, combinations thereof, and the like. In alternative embodiments, the physical and chemical properties of the materials, i.e., non-wetting low surface energy properties, can result in a passive process for ridding the surface of excess particle material. Next, substance 204 remaining in cavities 110 is hardened into particles 206 by, but is not limited to, photocuring, thermal curing, solvent evaporation, oxidation or reductive polymerization, change of temperature, crosslinking, nucleophilic substitution leading to crosslinking, combinations thereof, and the like. After substance 204 is hardened, the particles 206 are harvested from cavities 110.

According to some embodiments, surface 108 is configured such that particle fabrication is accomplished in high throughput. In some embodiments, the surface is configured, for example, planer, cylindrical, spherical, curved, linear, a conveyer belt type arrangement, a gravure printing type arrangement, in large sheet arrangements, in multi-layered sheet arrangements, combinations thereof, and the like.

Thus, the invention encompasses a method of forming a plurality of monodisperse pharmaceutical composition particles comprising introducing a solution having at least 25 weight % protein, at least 30, at least 40, at least 50, at least 60, at least 70, at least 75, at least 80, at least 90, at least 95 weight % protein into a plurality of cavities of a polymer mold, wherein the cavities have predetermined geometric shapes and a broadest dimension less than about 10 micrometers; and lyophilizing the aqueous solution within the cavities of the mold. The particles can be harvested from the cavities of the mold after lyophilizing. In harvesting, the particles can be removed onto a harvesting sheet. The particles may be arranged in an ordered array on the harvesting sheet, the ordered array mirroring an ordered array of the cavities of the mold.

III. Micro and/or Nano Pharmaceutical Organic Particles

According to some embodiments of the present invention, a pharmaceutical organic particle of the present invention is formed having a predetermined shape, size, formulation, density, composition, surface features, spectral analysis, modulus, hardness, or the like. In some embodiments, the predetermined size of the particle of the present invention can be less than or equal to about 10.00 μm in a broadest dimension (for example, but not as a limitation, the broadest dimension can be a maximum linear cross-sectional dimension, a maximum non-linear dimension, or the like). In some embodiments, the particle is less than or equal to about 7.50 μm in a broadest dimension. In some embodiments, the particle is less than or equal to about 5.00 μm in a broadest dimension. In alternative embodiments, the particle is less than or equal to about 4.99 μm in a broadest dimension, less than or equal to about 4.98 μm in a broadest dimension, less than or equal to about 4.97 μm in a broadest dimension, less than or equal to about 4.96 μm in a broadest dimension, less than or equal to about 4.95 μm in a broadest dimension, less than or equal to about 4.94 μm in a broadest dimension, less than or equal to about 4.93 μm in a broadest dimension, less than or equal to about 4.92 μm in a broadest dimension, less than or equal to about 4.91 μm in a broadest dimension, less than or equal to about 4.90 μm in a broadest dimension, less than or equal to about 4.89 μm in a broadest dimension, less than or equal to about 4.88 μm in a broadest dimension, less than or equal to about 4.87 μm in a broadest dimension, less than or equal to about 4.86 μm in a broadest dimension, less than or equal to about 4.85 μm in a broadest dimension, less than or equal to about 4.84 μm in a broadest dimension, less than or equal to about 4.83 μm in a broadest dimension, less than or equal to about 4.82 μm in a broadest dimension, less than or equal to about 4.81 μm in a broadest dimension, less than or equal to about 4.80 μm in a broadest dimension, less than or equal to about 4.79 μm in a broadest dimension, less than or equal to about 4.78 μm in a broadest dimension, less than or equal to about 4.77 μm in a broadest dimension, less than or equal to about 4.76 μm in a broadest dimension, less than or equal to about 4.75 μm in a broadest dimension, less than or equal to about 4.74 μm in a broadest dimension, less than or equal to about 4.73 μm in a broadest dimension, less than or equal to about 4.72 μm in a broadest dimension, less than or equal to about 4.71 μm in a broadest dimension, less than or equal to about 4.70 μm in a broadest dimension, less than or equal to about 4.69 μm in a broadest dimension, less than or equal to about 4.68 μm in a broadest dimension, less than or equal to about 4.67 μm in a broadest dimension, less than or equal to about 4.66 μm in a broadest dimension, less than or equal to about 4.65 μm in a broadest dimension, less than or equal to about 4.64 μm in a broadest dimension, less than or equal to about 4.63 μm in a broadest dimension, less than or equal to about 4.62 μm in a broadest dimension, less than or equal to about 4.61 μm in a broadest dimension, less than or equal to about 4.60 μm in a broadest dimension, less than or equal to about 4.59 μm in a broadest dimension, less than or equal to about 4.58 μm in a broadest dimension, less than or equal to about 4.57 μm in a broadest dimension, less than or equal to about 4.56 μm in a broadest dimension, less than or equal to about 4.55 μm in a broadest dimension, less than or equal to about 4.54 μm in a broadest dimension, less than or equal to about 4.53 μm in a broadest dimension, less than or equal to about 4.52 μm in a broadest dimension, less than or equal to about 4.51 μm in a broadest dimension, less than or equal to about 4.50 μm in a broadest dimension, less than or equal to about 4.49 μm in a broadest dimension, less than or equal to about 4.48 μm in a broadest dimension, less than or equal to about 4.47 μm in a broadest dimension, less than or equal to about 4.46 μm in a broadest dimension, less than or equal to about 4.45 μm in a broadest dimension, less than or equal to about 4.44 μm in a broadest dimension, less than or equal to about 4.43 μm in a broadest dimension, less than or equal to about 4.42 μm in a broadest dimension, less than or equal to about 4.41 μm in a broadest dimension, less than or equal to about 4.40 μm in a broadest dimension, less than or equal to about 4.39 μm in a broadest dimension, less than or equal to about 4.38 μm in a broadest dimension, less than or equal to about 4.37 μm in a broadest dimension, less than or equal to about 4.36 μm in a broadest dimension, less than or equal to about 4.35 μm in a broadest dimension, less than or equal to about 4.34 μm in a broadest dimension, less than or equal to about 4.33 μm in a broadest dimension, less than or equal to about 4.32 μm in a broadest dimension, less than or equal to about 4.31 μm in a broadest dimension, less than or equal to about 4.30 μm in a broadest dimension, less than or equal to about 4.29 μm in a broadest dimension, less than or equal to about 4.28 μm in a broadest dimension, less than or equal to about 4.27 μm in a broadest dimension, less than or equal to about 4.26 μm in a broadest dimension, less than or equal to about 4.25 μm in a broadest dimension, less than or equal to about 4.24 μm in a broadest dimension, less than or equal to about 4.23 μm in a broadest dimension, less than or equal to about 4.22 μm in a broadest dimension, less than or equal to about 4.21 μm in a broadest dimension, less than or equal to about 4.20 μm in a broadest dimension, less than or equal to about 4.19 μm in a broadest dimension, less than or equal to about 4.18 μm in a broadest dimension, less than or equal to about 4.17 μm in a broadest dimension, less than or equal to about 4.16 μm in a broadest dimension, less than or equal to about 4.15 μm in a broadest dimension, less than or equal to about 4.14 μm in a broadest dimension, less than or equal to about 4.13 μm in a broadest dimension, less than or equal to about 4.12 μm in a broadest dimension, less than or equal to about 4.11 μm in a broadest dimension, less than or equal to about 4.10 μm in a broadest dimension, less than or equal to about 4.09 μm in a broadest dimension, less than or equal to about 4.08 μm in a broadest dimension, less than or equal to about 4.07 μm in a broadest dimension, less than or equal to about 4.06 μm in a broadest dimension, less than or equal to about 4.05 μm in a broadest dimension, less than or equal to about 4.04 μm in a broadest dimension, less than or equal to about 4.03 μm in a broadest dimension, less than or equal to about 4.02 μm in a broadest dimension, less than or equal to about 4.01 μm in a broadest dimension, or less than or equal to about 4.00 μm in a broadest dimension.

In alternative embodiments, the predetermined size of the pharmaceutical organic particle is less than or equal to about 3.99 μm in a broadest dimension, less than or equal to about 3.98 μm in a broadest dimension, less than or equal to about 3.97 μm in a broadest dimension, less than or equal to about 3.96 μm in a broadest dimension, less than or equal to about 3.95 μm in a broadest dimension, less than or equal to about 3.94 μm in a broadest dimension, less than or equal to about 3.93 μm in a broadest dimension, less than or equal to about 3.92 μm in a broadest dimension, less than or equal to about 3.91 μm in a broadest dimension, less than or equal to about 3.90 μm in a broadest dimension, less than or equal to about 3.89 μm in a broadest dimension, less than or equal to about 3.88 μm in a broadest dimension, less than or equal to about 3.87 μm in a broadest dimension, less than or equal to about 3.86 μm in a broadest dimension, less than or equal to about 3.85 μm in a broadest dimension, less than or equal to about 3.84 μm in a broadest dimension, less than or equal to about 3.83 μm in a broadest dimension, less than or equal to about 3.82 μm in a broadest dimension, less than or equal to about 3.81 μm in a broadest dimension, less than or equal to about 3.80 μm in a broadest dimension, less than or equal to about 3.79 μm in a broadest dimension, less than or equal to about 3.78 μm in a broadest dimension, less than or equal to about 3.77 μm in a broadest dimension, less than or equal to about 3.76 μm in a broadest dimension, less than or equal to about 3.75 μm in a broadest dimension, less than or equal to about 3.74 μm in a broadest dimension, less than or equal to about 3.73 μm in a broadest dimension, less than or equal to about 3.72 μm in a broadest dimension, less than or equal to about 3.71 μm in a broadest dimension, less than or equal to about 3.70 μm in a broadest dimension, less than or equal to about 3.69 μm in a broadest dimension, less than or equal to about 3.68 μm in a broadest dimension, less than or equal to about 3.67 μm in a broadest dimension, less than or equal to about 3.66 μm in a broadest dimension, less than or equal to about 3.65 μm in a broadest dimension, less than or equal to about 3.64 μm in a broadest dimension, less than or equal to about 3.63 μm in a broadest dimension, less than or equal to about 3.62 μm in a broadest dimension, less than or equal to about 3.61 μm in a broadest dimension, less than or equal to about 3.60 μm in a broadest dimension, less than or equal to about 3.59 μm in a broadest dimension, less than or equal to about 3.58 μm in a broadest dimension, less than or equal to about 3.57 μm in a broadest dimension, less than or equal to about 3.56 μm in a broadest dimension, less than or equal to about 3.55 μm in a broadest dimension, less than or equal to about 3.54 μm in a broadest dimension, less than or equal to about 3.53 μm in a broadest dimension, less than or equal to about 3.52 μm in a broadest dimension, less than or equal to about 3.51 μm in a broadest dimension, less than or equal to about 3.50 μm in a broadest dimension, less than or equal to about 3.49 μm in a broadest dimension, less than or equal to about 3.48 μm in a broadest dimension, less than or equal to about 3.47 μm in a broadest dimension, less than or equal to about 3.46 μm in a broadest dimension, less than or equal to about 3.45 μm in a broadest dimension, less than or equal to about 3.44 μm in a broadest dimension, less than or equal to about 3.43 μm in a broadest dimension, less than or equal to about 3.42 μm in a broadest dimension, less than or equal to about 3.41 μm in a broadest dimension, less than or equal to about 3.40 μm in a broadest dimension, less than or equal to about 3.39 μm in a broadest dimension, less than or equal to about 3.38 μm in a broadest dimension, less than or equal to about 3.37 μm in a broadest dimension, less than or equal to about 3.36 μm in a broadest dimension, less than or equal to about 3.35 μm in a broadest dimension, less than or equal to about 3.34 μm in a broadest dimension, less than or equal to about 3.33 μm in a broadest dimension, less than or equal to about 3.32 μm in a broadest dimension, less than or equal to about 3.31 μm in a broadest dimension, less than or equal to about 3.30 μm in a broadest dimension, less than or equal to about 3.29 μm in a broadest dimension, less than or equal to about 3.28 μm in a broadest dimension, less than or equal to about 3.27 μm in a broadest dimension, less than or equal to about 3.26 μm in a broadest dimension, less than or equal to about 3.25 μm in a broadest dimension, less than or equal to about 3.24 μm in a broadest dimension, less than or equal to about 3.23 μm in a broadest dimension, less than or equal to about 3.22 μm in a broadest dimension, less than or equal to about 3.21 μm in a broadest dimension, less than or equal to about 3.20 μm in a broadest dimension, less than or equal to about 3.19 μm in a broadest dimension, less than or equal to about 3.18 μm in a broadest dimension, less than or equal to about 3.17 μm in a broadest dimension, less than or equal to about 3.16 μm in a broadest dimension, less than or equal to about 3.15 μm in a broadest dimension, less than or equal to about 3.14 μm in a broadest dimension, less than or equal to about 3.13 μm in a broadest dimension, less than or equal to about 3.12 μm in a broadest dimension, less than or equal to about 3.11 μm in a broadest dimension, less than or equal to about 3.10 μm in a broadest dimension, less than or equal to about 3.09 μm in a broadest dimension, less than or equal to about 3.08 μm in a broadest dimension, less than or equal to about 3.07 μm in a broadest dimension, less than or equal to about 3.06 μm in a broadest dimension, less than or equal to about 3.05 μm in a broadest dimension, less than or equal to about 3.04 μm in a broadest dimension, less than or equal to about 3.03 μm in a broadest dimension, less than or equal to about 3.02 μm in a broadest dimension, less than or equal to about 3.01 μm in a broadest dimension, or less than or equal to about 3.00 μm in a broadest dimension.

In alternative embodiments, the predetermined size of the pharmaceutical organic particle is less than or equal to about 2.99 μm in a broadest dimension, less than or equal to about 2.98 μm in a broadest dimension, less than or equal to about 2.97 μm in a broadest dimension, less than or equal to about 2.96 μm in a broadest dimension, less than or equal to about 2.95 μm in a broadest dimension, less than or equal to about 2.94 μm in a broadest dimension, less than or equal to about 2.93 μm in a broadest dimension, less than or equal to about 2.92 μm in a broadest dimension, less than or equal to about 2.91 μm in a broadest dimension, less than or equal to about 2.90 μm in a broadest dimension, less than or equal to about 2.89 μm in a broadest dimension, less than or equal to about 2.88 μm in a broadest dimension, less than or equal to about 2.87 μm in a broadest dimension, less than or equal to about 2.86 μm in a broadest dimension, less than or equal to about 2.85 μm in a broadest dimension, less than or equal to about 2.84 μm in a broadest dimension, less than or equal to about 2.83 μm in a broadest dimension, less than or equal to about 2.82 μm in a broadest dimension, less than or equal to about 2.81 μm in a broadest dimension, less than or equal to about 2.80 μm in a broadest dimension, less than or equal to about 2.79 μm in a broadest dimension, less than or equal to about 2.78 μm in a broadest dimension, less than or equal to about 2.77 μm in a broadest dimension, less than or equal to about 2.76 μm in a broadest dimension, less than or equal to about 2.75 μm in a broadest dimension, less than or equal to about 2.74 μm in a broadest dimension, less than or equal to about 2.73 μm in a broadest dimension, less than or equal to about 2.72 μm in a broadest dimension, less than or equal to about 2.71 μm in a broadest dimension, less than or equal to about 2.70 μm in a broadest dimension, less than or equal to about 2.69 μm in a broadest dimension, less than or equal to about 2.68 μm in a broadest dimension, less than or equal to about 2.67 μm in a broadest dimension, less than or equal to about 2.66 μm in a broadest dimension, less than or equal to about 2.65 μm in a broadest dimension, less than or equal to about 2.64 μm in a broadest dimension, less than or equal to about 2.63 μm in a broadest dimension, less than or equal to about 2.62 μm in a broadest dimension, less than or equal to about 2.61 μm in a broadest dimension, less than or equal to about 2.60 μm in a broadest dimension, less than or equal to about 2.59 μm in a broadest dimension, less than or equal to about 2.58 μm in a broadest dimension, less than or equal to about 2.57 μm in a broadest dimension, less than or equal to about 2.56 μm in a broadest dimension, less than or equal to about 2.55 μm in a broadest dimension, less than or equal to about 2.54 μm in a broadest dimension, less than or equal to about 2.53 μm in a broadest dimension, less than or equal to about 2.52 μm in a broadest dimension, less than or equal to about 2.51 μm in a broadest dimension, less than or equal to about 2.50 μm in a broadest dimension, less than or equal to about 2.49 μm in a broadest dimension, less than or equal to about 2.48 μm in a broadest dimension, less than or equal to about 2.47 μm in a broadest dimension, less than or equal to about 2.46 μm in a broadest dimension, less than or equal to about 2.45 μm in a broadest dimension, less than or equal to about 2.44 μm in a broadest dimension, less than or equal to about 2.43 μm in a broadest dimension, less than or equal to about 2.42 μm in a broadest dimension, less than or equal to about 2.41 μm in a broadest dimension, less than or equal to about 2.40 μm in a broadest dimension, less than or equal to about 2.39 μm in a broadest dimension, less than or equal to about 2.38 μm in a broadest dimension, less than or equal to about 2.37 μm in a broadest dimension, less than or equal to about 2.36 μm in a broadest dimension, less than or equal to about 2.35 μm in a broadest dimension, less than or equal to about 2.34 μm in a broadest dimension, less than or equal to about 2.33 μm in a broadest dimension, less than or equal to about 2.32 μm in a broadest dimension, less than or equal to about 2.31 μm in a broadest dimension, less than or equal to about 2.30 μm in a broadest dimension, less than or equal to about 2.29 μm in a broadest dimension, less than or equal to about 2.28 μm in a broadest dimension, less than or equal to about 2.27 μm in a broadest dimension, less than or equal to about 2.26 μm in a broadest dimension, less than or equal to about 2.25 μm in a broadest dimension, less than or equal to about 2.24 μm in a broadest dimension, less than or equal to about 2.23 μm in a broadest dimension, less than or equal to about 2.22 μm in a broadest dimension, less than or equal to about 2.21 μm in a broadest dimension, less than or equal to about 2.20 μm in a broadest dimension, less than or equal to about 2.19 μm in a broadest dimension, less than or equal to about 2.18 μm in a broadest dimension, less than or equal to about 2.17 μm in a broadest dimension, less than or equal to about 2.16 μm in a broadest dimension, less than or equal to about 2.15 μm in a broadest dimension, less than or equal to about 2.14 μm in a broadest dimension, less than or equal to about 2.13 μm in a broadest dimension, less than or equal to about 2.12 μm in a broadest dimension, less than or equal to about 2.11 μm in a broadest dimension, less than or equal to about 2.10 μm in a broadest dimension, less than or equal to about 2.09 μm in a broadest dimension, less than or equal to about 2.08 μm in a broadest dimension, less than or equal to about 2.07 μm in a broadest dimension, less than or equal to about 2.06 μm in a broadest dimension, less than or equal to about 2.05 μm in a broadest dimension, less than or equal to about 2.04 μm in a broadest dimension, less than or equal to about 2.03 μm in a broadest dimension, less than or equal to about 2.02 μm in a broadest dimension, less than or equal to about 2.01 μm in a broadest dimension, or less than or equal to about 2.00 μm in a broadest dimension.

In alternative embodiments, the predetermined size of the pharmaceutical organic particle is less than or equal to about 1.99 μm in a broadest dimension, less than or equal to about 1.98 μm in a broadest dimension, less than or equal to about 1.97 μm in a broadest dimension, less than or equal to about 1.96 μm in a broadest dimension, less than or equal to about 1.95 μm in a broadest dimension, less than or equal to about 1.94 μm in a broadest dimension, less than or equal to about 1.93 μm in a broadest dimension, less than or equal to about 1.92 μm in a broadest dimension, less than or equal to about 1.91 μm in a broadest dimension, less than or equal to about 1.90 μm in a broadest dimension, less than or equal to about 1.89 μm in a broadest dimension, less than or equal to about 1.88 μm in a broadest dimension, less than or equal to about 1.87 μm in a broadest dimension, less than or equal to about 1.86 μm in a broadest dimension, less than or equal to about 1.85 μm in a broadest dimension, less than or equal to about 1.84 μm in a broadest dimension, less than or equal to about 1.83 μm in a broadest dimension, less than or equal to about 1.82 μm in a broadest dimension, less than or equal to about 1.81 μm in a broadest dimension, less than or equal to about 1.80 μm in a broadest dimension, less than or equal to about 1.79 μm in a broadest dimension, less than or equal to about 1.78 μm in a broadest dimension, less than or equal to about 1.77 μm in a broadest dimension, less than or equal to about 1.76 μm in a broadest dimension, less than or equal to about 1.75 μm in a broadest dimension, less than or equal to about 1.74 μm in a broadest dimension, less than or equal to about 1.73 μm in a broadest dimension, less than or equal to about 1.72 μm in a broadest dimension, less than or equal to about 1.71 μm in a broadest dimension, less than or equal to about 1.70 μm in a broadest dimension, less than or equal to about 1.69 μm in a broadest dimension, less than or equal to about 1.68 μm in a broadest dimension, less than or equal to about 1.67 μm in a broadest dimension, less than or equal to about 1.66 μm in a broadest dimension, less than or equal to about 1.65 μm in a broadest dimension, less than or equal to about 1.64 μm in a broadest dimension, less than or equal to about 1.63 μm in a broadest dimension, less than or equal to about 1.62 μm in a broadest dimension, less than or equal to about 1.61 μm in a broadest dimension, less than or equal to about 1.60 μm in a broadest dimension, less than or equal to about 1.59 μm in a broadest dimension, less than or equal to about 1.58 μm in a broadest dimension, less than or equal to about 1.57 μm in a broadest dimension, less than or equal to about 1.56 μm in a broadest dimension, less than or equal to about 1.55 μm in a broadest dimension, less than or equal to about 1.54 μm in a broadest dimension, less than or equal to about 1.53 μm in a broadest dimension, less than or equal to about 1.52 μm in a broadest dimension, less than or equal to about 1.51 μm in a broadest dimension, less than or equal to about 1.50 μm in a broadest dimension, less than or equal to about 1.49 μm in a broadest dimension, less than or equal to about 1.48 μm in a broadest dimension, less than or equal to about 1.47 μm in a broadest dimension, less than or equal to about 1.46 μm in a broadest dimension, less than or equal to about 1.45 μm in a broadest dimension, less than or equal to about 1.44 μm in a broadest dimension, less than or equal to about 1.43 μm in a broadest dimension, less than or equal to about 1.42 μm in a broadest dimension, less than or equal to about 1.41 μm in a broadest dimension, less than or equal to about 1.40 μm in a broadest dimension, less than or equal to about 1.39 μm in a broadest dimension, less than or equal to about 1.38 μm in a broadest dimension, less than or equal to about 1.37 μm in a broadest dimension, less than or equal to about 1.36 μm in a broadest dimension, less than or equal to about 1.35 μm in a broadest dimension, less than or equal to about 1.34 μm in a broadest dimension, less than or equal to about 1.33 μm in a broadest dimension, less than or equal to about 1.32 μm in a broadest dimension, less than or equal to about 1.31 μm in a broadest dimension, less than or equal to about 1.30 μm in a broadest dimension, less than or equal to about 1.29 μm in a broadest dimension, less than or equal to about 1.28 μm in a broadest dimension, less than or equal to about 1.27 μm in a broadest dimension, less than or equal to about 1.26 μm in a broadest dimension, less than or equal to about 1.25 μm in a broadest dimension, less than or equal to about 1.24 μm in a broadest dimension, less than or equal to about 1.23 μm in a broadest dimension, less than or equal to about 1.22 μm in a broadest dimension, less than or equal to about 1.21 μm in a broadest dimension, less than or equal to about 1.20 μm in a broadest dimension, less than or equal to about 1.19 μm in a broadest dimension, less than or equal to about 1.18 μm in a broadest dimension, less than or equal to about 1.17 μm in a broadest dimension, less than or equal to about 1.16 μm in a broadest dimension, less than or equal to about 1.15 μm in a broadest dimension, less than or equal to about 1.14 μm in a broadest dimension, less than or equal to about 1.13 μm in a broadest dimension, less than or equal to about 1.12 μm in a broadest dimension, less than or equal to about 1.11 μm in a broadest dimension, less than or equal to about 1.10 μm in a broadest dimension, less than or equal to about 1.09 μm in a broadest dimension, less than or equal to about 1.08 μm in a broadest dimension, less than or equal to about 1.07 μm in a broadest dimension, less than or equal to about 1.06 μm in a broadest dimension, less than or equal to about 1.05 μm in a broadest dimension, less than or equal to about 1.04 μm in a broadest dimension, less than or equal to about 1.03 μm in a broadest dimension, less than or equal to about 1.02 μm in a broadest dimension, less than or equal to about 1.01 μm in a broadest dimension, or less than or equal to about 1.00 μm in a broadest dimension.

In alternative embodiments, the predetermined size of the pharmaceutical organic particle is less than or equal to about 990 nm in a broadest dimension, less than or equal to about 980 nm in a broadest dimension, less than or equal to about 970 nm in a broadest dimension, less than or equal to about 960 nm in a broadest dimension, less than or equal to about 950 nm in a broadest dimension, less than or equal to about 940 nm in a broadest dimension, less than or equal to about 930 nm in a broadest dimension, less than or equal to about 920 nm in a broadest dimension, less than or equal to about 910 nm in a broadest dimension, less than or equal to about 900 nm in a broadest dimension, less than or equal to about 890 nm in a broadest dimension, less than or equal to about 880 nm in a broadest dimension, less than or equal to about 870 nm in a broadest dimension, less than or equal to about 860 nm in a broadest dimension, less than or equal to about 850 nm in a broadest dimension, less than or equal to about 840 nm in a broadest dimension, less than or equal to about 830 nm in a broadest dimension, less than or equal to about 820 nm in a broadest dimension, less than or equal to about 810 nm in a broadest dimension, less than or equal to about 800 nm in a broadest dimension, less than or equal to about 790 nm in a broadest dimension, less than or equal to about 780 nm in a broadest dimension, less than or equal to about 770 nm in a broadest dimension, less than or equal to about 760 nm in a broadest dimension, less than or equal to about 750 nm in a broadest dimension, less than or equal to about 740 nm in a broadest dimension, less than or equal to about 730 nm in a broadest dimension, less than or equal to about 720 nm in a broadest dimension, less than or equal to about 710 nm in a broadest dimension, less than or equal to about 700 nm in a broadest dimension, less than or equal to about 690 nm in a broadest dimension, less than or equal to about 680 nm in a broadest dimension, less than or equal to about 670 nm in a broadest dimension, less than or equal to about 660 nm in a broadest dimension, less than or equal to about 650 nm in a broadest dimension, less than or equal to about 640 nm in a broadest dimension, less than or equal to about 630 nm in a broadest dimension, less than or equal to about 620 nm in a broadest dimension, less than or equal to about 610 nm in a broadest dimension, less than or equal to about 600 nm in a broadest dimension, less than or equal to about 590 nm in a broadest dimension, less than or equal to about 580 nm in a broadest dimension, less than or equal to about 570 nm in a broadest dimension, less than or equal to about 560 nm in a broadest dimension, less than or equal to about 550 nm in a broadest dimension, less than or equal to about 540 nm in a broadest dimension, less than or equal to about 530 nm in a broadest dimension, less than or equal to about 520 nm in a broadest dimension, less than or equal to about 510 nm in a broadest dimension, less than or equal to about 500 nm in a broadest dimension, less than or equal to about 490 nm in a broadest dimension, less than or equal to about 480 nm in a broadest dimension, less than or equal to about 470 nm in a broadest dimension, less than or equal to about 460 nm in a broadest dimension, less than or equal to about 450 nm in a broadest dimension, less than or equal to about 440 nm in a broadest dimension, less than or equal to about 430 nm in a broadest dimension, less than or equal to about 420 nm in a broadest dimension, less than or equal to about 410 nm in a broadest dimension, less than or equal to about 400 nm in a broadest dimension, less than or equal to about 390 nm in a broadest dimension, less than or equal to about 380 nm in a broadest dimension, less than or equal to about 370 nm in a broadest dimension, less than or equal to about 360 nm in a broadest dimension, less than or equal to about 350 nm in a broadest dimension, less than or equal to about 340 nm in a broadest dimension, less than or equal to about 330 nm in a broadest dimension, less than or equal to about 320 nm in a broadest dimension, less than or equal to about 310 nm in a broadest dimension, less than or equal to about 300 nm in a broadest dimension, less than or equal to about 290 nm in a broadest dimension, less than or equal to about 280 nm in a broadest dimension, less than or equal to about 270 nm in a broadest dimension, less than or equal to about 260 nm in a broadest dimension, less than or equal to about 250 nm in a broadest dimension, less than or equal to about 240 nm in a broadest dimension, less than or equal to about 230 nm in a broadest dimension, less than or equal to about 220 nm in a broadest dimension, less than or equal to about 210 nm in a broadest dimension, less than or equal to about 200 nm in a broadest dimension, less than or equal to about 190 nm in a broadest dimension, less than or equal to about 180 nm in a broadest dimension, less than or equal to about 170 nm in a broadest dimension, less than or equal to about 160 nm in a broadest dimension, less than or equal to about 150 nm in a broadest dimension, less than or equal to about 140 nm in a broadest dimension, less than or equal to about 130 nm in a broadest dimension, less than or equal to about 120 nm in a broadest dimension, less than or equal to about 110 nm in a broadest dimension, less than or equal to about 100 nm in a broadest dimension, less than or equal to about 90 nm in a broadest dimension, less than or equal to about 80 nm in a broadest dimension, less than or equal to about 70 nm in a broadest dimension, less than or equal to about 60 nm in a broadest dimension, less than or equal to about 50 nm in a broadest dimension, less than or equal to about 40 nm in a broadest dimension, less than or equal to about 30 nm in a broadest dimension, less than or equal to about 20 nm in a broadest dimension, or less than or equal to about 10 nm in a broadest dimension.

In an alternative embodiment, the predetermined size of the pharmaceutical organic particle of the present invention is between about 5.00 μm and about 0.25 μm in a broadest dimension. In another embodiment, the predetermined size of the pharmaceutical organic particle is between about 4.75 μm and about 0.25 μm in a broadest dimension. In another embodiment, the predetermined size of the pharmaceutical organic particle is between about 4.50 μm and about 0.25 μm in a broadest dimension. In another embodiment, the predetermined size of the pharmaceutical organic particle is between about 4.25 μm and about 0.25 μm in a broadest dimension. In another embodiment, the predetermined size of the pharmaceutical organic particle is between about 4.00 μm and about 0.25 μm in a broadest dimension. In another embodiment, the predetermined size of the pharmaceutical organic particle is between about 3.75 μm and about 0.25 μm in a broadest dimension. In another embodiment, the predetermined size of the pharmaceutical organic particle is between about 3.50 μm and about 0.25 μm in a broadest dimension. In another embodiment, the predetermined size of the pharmaceutical organic particle is between about 3.25 μm and about 0.25 μm in a broadest dimension. In another embodiment, the predetermined size of the pharmaceutical organic particle is between about 3.00 μm and about 0.25 μm in a broadest dimension. In another embodiment, the predetermined size of the pharmaceutical organic particle is between about 2.75 μm and about 0.25 μm in a broadest dimension. In another embodiment, the predetermined size of the pharmaceutical organic particle is between about 2.50 μm and about 0.25 μm in a broadest dimension. In another embodiment, the predetermined size of the pharmaceutical organic particle is between about 2.25 μm and about 0.25 μm in a broadest dimension. In another embodiment, the predetermined size of the pharmaceutical organic particle is between about 2.00 μm and about 0.25 μm in a broadest dimension. In another embodiment, the predetermined size of the pharmaceutical organic particle is between about 1.75 μm and about 0.25 μm in a broadest dimension. In another embodiment, the predetermined size of the pharmaceutical organic particle is between about 1.50 μm and about 0.25 μm in a broadest dimension. In another embodiment, the predetermined size of the pharmaceutical organic particle is between about 1.25 μm and about 0.25 μm in a broadest dimension. In another embodiment, the predetermined size of the pharmaceutical organic particle is between about 1.00 μm and about 0.25 μm in a broadest dimension. In another embodiment, the predetermined size of the pharmaceutical organic particle is between about 0.75 μm and about 0.25 μm in a broadest dimension. In another embodiment, the predetermined size of the pharmaceutical organic particle is between about 0.50 μm and about 0.25 μm in a broadest dimension.

In another embodiment, the predetermined size of the pharmaceutical organic particle is between about 5.00 μm and about 10 nm in a broadest dimension. In another embodiment, the particle is between about 4.50 μm and about 10 nm in a broadest dimension. In another embodiment, the particle is between about 4.00 μm and about 10 nm in a broadest dimension. In another embodiment, the particle is between about 3.50 μm and about 10 nm in a broadest dimension. In another embodiment, the particle is between about 3.00 μm and about 10 nm in a broadest dimension. In another embodiment, the particle is between about 2.50 μm and about 10 nm in a broadest dimension. In another embodiment, the particle is between about 2.00 μm and about 10 nm in a broadest dimension. In another embodiment, the particle is between about 1.50 μm and about 10 nm in a broadest dimension. In another embodiment, the particle is between about 1.00 μm and about 10 nm in a broadest dimension. In another embodiment, the particle is between about 0.50 μm and about 10 nm in a broadest dimension. In another embodiment, the particle is between about 0.25 μm and about 10 nm in a broadest dimension. In another embodiment, the particle is between about 0.20 μm and about 10 nm in a broadest dimension. In another embodiment, the particle is between about 0.15 μm and about 10 nm in a broadest dimension. In another embodiment, the particle is between about 0.10 μm and about 10 nm in a broadest dimension. In another embodiment, the particle is between about 75 nm and about 10 nm in a broadest dimension. In another embodiment, the particle is between about 50 nm and about 10 nm in a broadest dimension. In another embodiment, the particle is between about 25 nm and about 10 nm in a broadest dimension.

According to other embodiments, particles of many predetermined regular and irregular shape and size configurations can be made with the materials and methods of the presently disclosed subject matter. Examples of representative shapes that can be made using the materials and methods of the presently disclosed subject matter include, but are not limited to, non-spherical, spherical, cubed, columnar, cylindrical, cone shaped, viral shaped, bacteria shaped, cell shaped, rod shaped, chiral shaped, right triangle shaped, flat disc shaped, boomerang shaped, combinations thereof, and the like.

The particle can be of an organic material, an inorganic material, a pharmaceutical, a composite, or the like and can be one uniform compound or component or a mixture of compounds or components.

IV. Particle Dispersity Measurements

According to other embodiments, the particles produced by the methods and materials of the present subject matter have substantially the same size and/or three-dimensional geometric shape. By “substantially the same geometric shape,” the particles are substantially equivalent in any lateral dimension, cross section, volume, mass, surface area and the like. That is, more than about 90%, about 95% of the particles have the same geometric shape.

In general, measurement or quantification of unimodal or multimodal size distribution of samples can be calculated using dynamic light scattering (DLS) techniques. Dynamic light scattering (DLS) measures the intensity fluctuations with time and correlates these fluctuations, through algorithmic calculation, to the properties of the scattering objects, presented as autocorrelation function g⁽²⁾(q,t) of the scattering intensity. The autocorrelation function depends on how molecules move on the length scale 1/q, with a characteristic time τ;

$$τ = \frac{1}{{Dq}^{2}}$$

where D is the transitional diffusion coefficient.

The scattering wave vector q is given by

$$q = \frac{4 π {n}_{s}}{λ} sin ( Θ / 2 )$$

where nₛ is the refractive index of the solvent, λ is the wavelength of the light in the vacuum and Θ is the scattering angle.

The particle sizes are calculated from transitional diffusion coefficient by Stroke-Einstein Equation;

$$D = \frac{{k}_{B} T}{3 π η ( t ) d}$$

where ηₛ is the solvent viscosity, k_{B} is the Boltzmann constant, T is the absolute temperature and Rₕ is the hydrodynamic radius.

According to an embodiment of the present invention, for samples with broad unimodal or multimodal size distribution, DLS data was analyzed by the Non-Negative Constrained Lease Squares (NNLS) (I. Morrison, E. Grabowski, and C. Herb, Langmuir, 1 (1985) 496) and integral transform method CONTIN (S. Provencher, Computer Phys. Comm. 27 (1982) 213 and 229), each of which is incorporated herein by reference in its entirety, to obtain size and size distribution.

The polydispersity of particles were calculated by Cumulant Analysis (D. Koppel, J. Chem. Phys., 57 (1972) 4814), which is incorporated herein by reference. The statistic deviation of diffusion coefficient is (based on the band width of lognormal plot):

$$Polydispersity = {μ}_{2} / {τ}^{2} = \frac{( {D}^{2} - {\overline{D}}^{2} )}{{D}^{2}}$$

where μ₂ is proportional to the variations of the “intensity” weighed diffusion coefficient distribution and carries the information of the width of the size distribution; D̅ is the average diffusion coefficient.

Polydispersity has no unit and has been reported as the indication of size distribution of colloids or particles. A general characteristic of dispersion size distributions is shown in Table 1, where a dispersity below 0.02 is typically accepted as indicating a monodisperse sample, between 0.02 and 0.08 is typically accepted as a narrow disperse sample distribution, and above 0.08 is typically accepted as a sample having broad disperse size distribution among the sample.

TABLE 1

| Polydispersity | Interpretation |
|---|---|
| 0-0.02 | Monodisperse |
| 0.02-0.08 | narrow disperse |
| >0.08 | broad disperse |

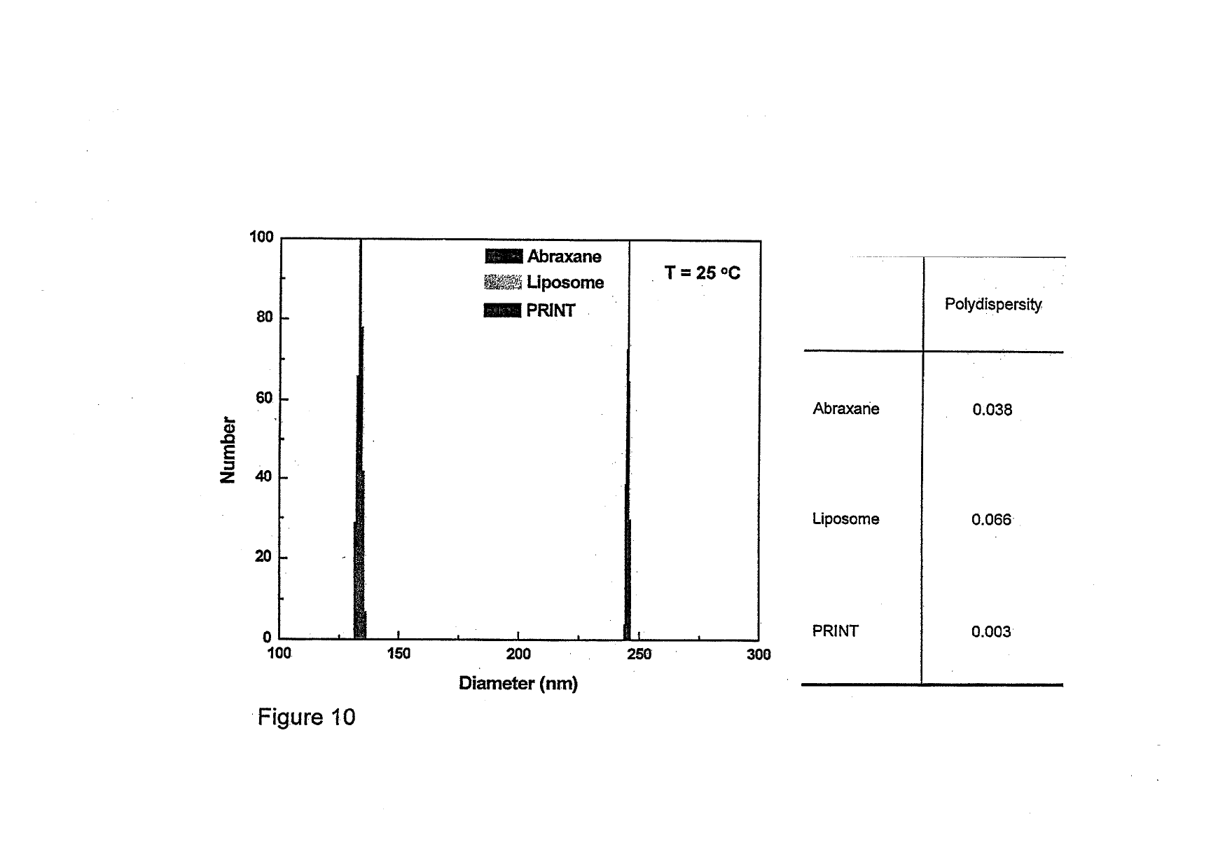

In some embodiments, PRINT particles fabricated according to methods and materials disclosed herein can include a polydispersity index of about 0.003, as shown in FIG. 10. In other embodiments, particles fabricated according to methods disclosed herein can have a polydispersity index of less than about 0.005. In other embodiments, particles fabricated according to methods disclosed herein can have a polydispersity index of less than about 0.007. In other embodiments, particles fabricated according to methods disclosed herein can have a polydispersity index of less than about 0.010. In other embodiments, particles fabricated according to methods disclosed herein can have a polydispersity index of less than about 0.015.

V. Particle Compositions

According to the present invention, pharmaceutical organic particles of the present invention are protein particles. In some instances the protein is a therapeutic or diagnostic protein and while other active agents may be present they are not necessary. In other instances, the protein is a carrier and the particles will additionally comprise at least one active agent. As discussed above, a structural component of the micro and/or nanoparticles of the invention is protein. Depending upon the protein used in the making of the particle and the therapeutic or diagnostic use, the protein component of the micro and/or nanoparticles will vary from at least about 20% up to about 100% by weight of the particle. The protein may be combined with buffers or other excipients prior to formation of the particles.

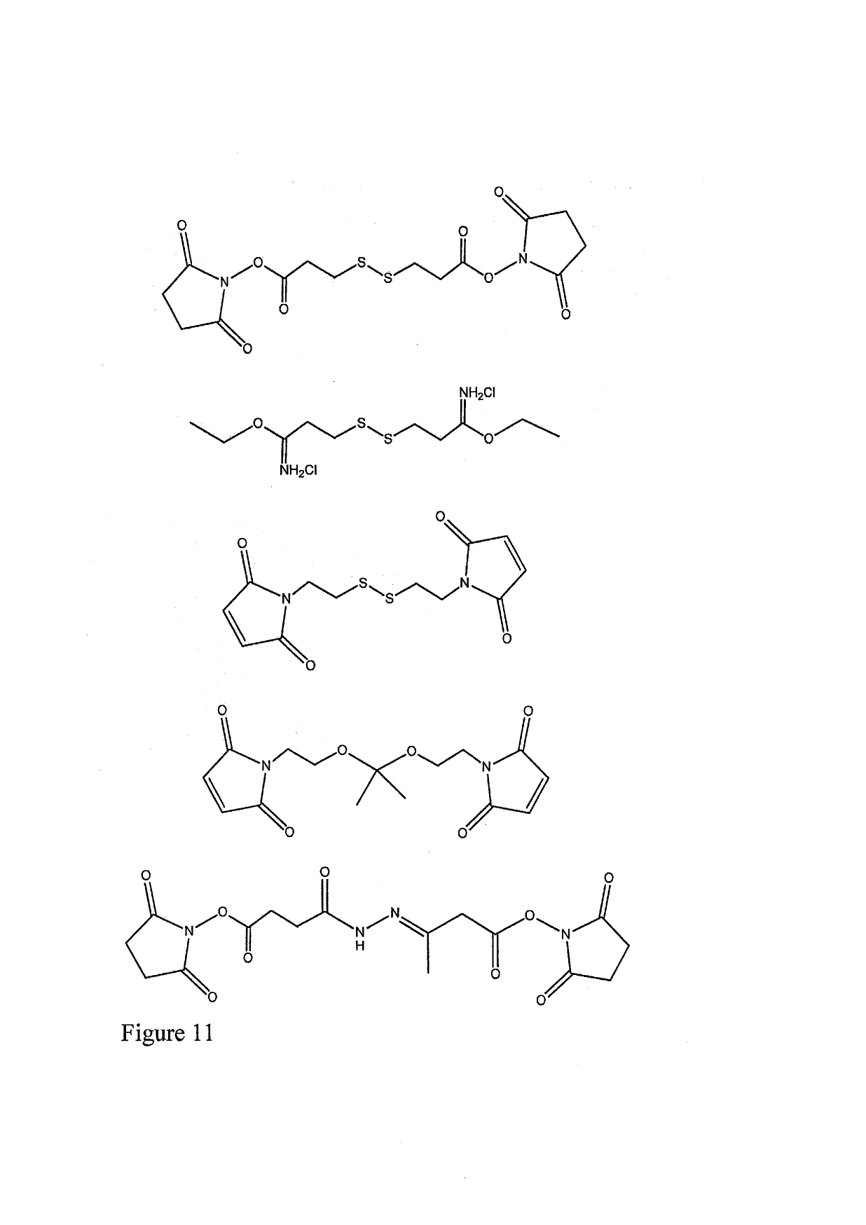

In some embodiments, the pharmaceutical organic particles of the present invention can be treated to modify or otherwise alter the protein component to degrade under specific conditions such that the active pharmaceutical agent is preserved. According to some embodiments, as the pharmaceutical agent is preserved until the particle reaches a target environment, the pharmaceutical agent can treat a specific site at levels that could not be systemically administered. In this manner, the proteins can be chemically crosslinked either through the nitrogen functionality of N-terminus of the lysine residues or the thiol functionality of cysteine residues using an appropriate difunctional crosslinker, e.g. glutaraldehyde or bis-maleimidohexane which will degrade under a reducing environment. In other embodiments, the crosslinkers can also have a functional group between the two protein reactive groups that can degrade under specific in vivo conditions, such as for example, disulfide, acetal, ketal, or hydrazone, as shown in FIG. 11. The acetal and ketal crosslinkers will degrade under an acidic environment. According to some embodiments, the surface crosslinking of the particles with degradable crosslinks can allow an entrapped cargo to remain within the particle and maintain particle size and shape until the particle is endocytosed by a cell. Temperature sensitive materials can be incorporated in the particles of the present invention include, but are not limited to, copolymers of polyacrylamides or copolymers of polyalkylene glycols and polylactide/glycolides, combinations thereof, and the like. In one example, albumin can be crosslinked through the lone thiol with a crosslinker that includes a labile or sensitive functional group within the compound such as a disulfide or ketal.

i) Polymer Component

Several biocompatible materials may be employed as the non-active pharmaceutical component or carrier of the therapeutic organic particles or protein micro and/or nanoparticles of the present invention. In some embodiments, naturally occurring biocompatible materials that can be utilized in the present invention include, but are not limited to: proteins; polypeptides; oligopeptides; polynucleotides; polysaccharides such as starch, cellulose, dextrans, alginates, chitosan, pectin, hyaluronic acid, and the like; lipids; combinations thereof; and the like. Examples of suitable proteins include albumin, insulin, hemoglobin, lysozyme, immunoglobulins, alpha-2-macroglobulin, fibronectin, vitronectin, fibrinogen, casein, transferrin, interferon-beta combinations thereof, and the like. According to an embodiment of the present invention, albumin is utilized as the protein component of the particles. In other embodiments, proteins such as a-2-macroglobulin, an opsonin, can be used to enhance cellular uptake of the particles of the present invention by macrophase-like cells of the liver and spleen. In other embodiments, ligands, such as glycoproteins, may also enhance uptake into certain tissues. In yet other embodiments, transferring can serve as a marker for clatherin-mediated endocytosis thereby improving uptake. In yet further embodiments, other functional proteins, such as antibodies and enzymes (including horse radish peroxidase, trypsin), can facilitate targeting of a biologic to a desired site and can be combined with the particles of the present invention. In other embodiments, protease can be utilized to help degrade a particle once the target environment is reached. In alternative embodiments, suitable biocompatible polymers for utilization in the organic pharmaceutical particle of the present invention include naturally occurring or synthetic proteins, provided such proteins have sufficient cysteine residues, or the like, within their amino acid sequences so that cross-linking can occur.

In other embodiments, synthetic polymers can be included in the pharmaceutical organic particles of the present invention. According to such embodiments, some examples include, but are not limited to synthetic polypeptides containing cysteine residues, linear or branched chain polyalkylene glycols, polyvinyl alcohol, polyacrylates, polyhydroxyethyl methacrylate, polyacrylic acid, polyethyloxazoline, polyacrylamides, polyisopropyl acrylamides, polyvinyl pyrrolidinone, polylactide/glycolide, combinations thereof, and the like. According to further embodiments, synthetic polymers useful in combination with the particles of the present invention include, but are not limited to, synthetic polyamino acids containing cysteine residues, synthetic polyamino acids containing disulfide groups, polyvinyl alcohol modified to contain free sulfhydryl groups, polyvinyl alcohol modified to contain free disulfide groups, polyhydroxyethyl methacrylate modified to contain free sulfhydryl groups, polyhydroxyethyl methacrylate modified to contain free disulfide groups, polyacrylic acid modified to contain free sulfhydryl groups, polyacrylic acid modified to contain free disulfide groups, polyethyloxazoline modified to contain free sulfhydryl groups, polyethyloxazoline modified to contain free disulfide groups, polyacrylamide modified to contain free sulfhydryl groups, polyacrylamide modified to contain free disulfide groups, polyvinyl pyrrolidinone modified to contain free sulfhydryl groups, polyvinyl pyrrolidinone modified to contain free disulfide groups, polyalkylene glycols modified to contain free sulfhydryl groups, polyalkylene glycols modified to contain free disulfide groups, combinations thereof, and the like.

In alternative embodiments, the protein component of the particle composition can undergo modifications, such as, but not limited to modifications of albumin through the amino acid residue, disulfide linkage, carboxylic acid residue, primary amine, free thiol, combinations thereof, and the like.

In further embodiments, polymers useful for including in the particles of the present invention include PEG containing sulfhydryl groups.

In further embodiments, transferrin, a serum protein typically responsible for delivering iron to cells within the body can be utilized as the protein or polymer component of the pharmaceutical organic particle of the present invention. The transport of iron is tightly regulated, but the high metabolic rate of cancerous cells causes increased demand for iron, which results in the over expression of transferrin-receptor on the cellular surface. The overexpression of this receptor makes transferrin an ideal targeting moiety for selective delivery of chemotherapeutics, and there are many examples of transferrin targeted delivery. It also has been shown that use of transferrin oligomers can increase endosomal residency time, which can aid in using transferrin in drug delivery applications. Currently, Transmid (Xenova Ltd), transferrin conjugated diphtheria toxin, is in phase III clinical trials for treating glioblastoma multiforme. Further description of transferrin can be found in Ching-Jou Lim and Wei-Chiang Shen. “Transferrin-Oligomers as Potential Carriers in Anticancer Drug Delivery,” Pharmaceutical Research, Vol. 21, No. 11, November 2004, 1985-1992; Hisae Inumai, Kazuo Maruyama, Kota Okinaga, Katsunori Sasaki, Toshiyuki Sekine, Osamu Ishida, Naoko Ogiwara, Kohei Johkura and Yutaka Yonemura, “Intracellular Targeting Therapy Of Cisplatin-Encapsulated Transferrin-Polyethylene Glycol Liposome On Peritoneal Dissemination Of Gastric Cancer,” Int. J. Cancer: 99, 130-137 (2002); Pei-Hui Yang, Xuesong Sun, Jen-Fu Chiu, Hongzhe Sun, and Qing-Yu He. “Transferrin-Mediated Gold Nanoparticle Cellular Uptake,” Bioconjugate Chem. 2005, 16, 494-496; and Nathalie C. Bellocq, Suzie H. Pun, Gregory S. Jensen, and Mark E. Davis “Transferrin-Containing, Cyclodextrin Polymer-Based Particles for Tumor-Targeted Gene Delivery,” Bioconjugate Chem. 2003, 14, 1122-1132; each of which is incorporated herein by reference in its entirety.

ii) Pharmaceutical Component

In some embodiments, the pharmaceutical organic particles of the present invention include an active pharmaceutical agent. In some embodiments, the pharmaceutically active agent can be combined with the polymer component to form a particle precursor material. According to such embodiments, the particle precursor material is then introduced to cavities of the molds and formed into micro or nanoparticles disclosed herein. According to some embodiments, the particle precursor material can include the polymer component without the pharmaceutically active agent. According to embodiments where the particle precursor material does not include the pharmaceutically active agent, the active agent can be introduced into the particle after the particle is fabricated. For example, in some embodiments polymer particles composed of, but not limited to, a protein such as albumin, unmodified or modified by, but not limited to, chemical crosslinking can be exposed to a concentrated solution containing, but not limited to, a pharmacologically active agent, such as taxol. The exposure provides diffusion of the active agent into the particles. Further discussion of diffusing active agents into polymer particles is described in lemma, F. et al., Radical Cross-Linked Albumin Microspheres as Potential Drug Delivery Systems: Preparation and In Vitro Studies, Drug Delivery, 12:229-234, (2005), which is incorporated herein by reference in its entirety.

In some embodiments, pharmacologically active agents useful in combining with the particles of the present invention include taxanes, such as paclitaxel for example; siRNA; doxorubicin; rapamyacin; sirolimus; antisense oligonucleotides; enzymes; protease; other chemotherapeutics; antiinfective agents; immunosuppressive agents; ions; minerals, such as calcium or potassium; contrast agents; combinations thereof; and the like. Details of further taxanes are discussed in the Physician Desk Reference, 1999, which is incorporated herein by reference in its entirety. In further embodiments, broad classes of compounds such as apoptosis inducing agents, antimitotic agents, microtubule or tubulin binding agents, taxanes, epothilones, COX-2 inhibitors, protease inhibitors, natural products of marine origin and their derivatives, marine polyketides such as discodermolide, eleutherobin, sarcodictyin A, combinations thereof, and the like, in addition to compounds referenced in related patent applications incorporated herein, can be combined with the polymer component of the particles of the present invention to form the pharmaceutical organic particle. Still further agents useful with the particles of the present invention include epothilones and antitumor agents useful for the invention described in an article by Nicolaou et al. (Angew. Chem. Int. Ed. 1998, 37, 2014-2045), which is incorporated herein by reference in its entirety.

In some embodiments, substantially water insoluble pharmacologically active agents useful in combining or adding to the polymer component of the particles of the present invention include, but are not limited to taxol (as used herein, the term “taxol” is intended to include taxol analogs and prodrugs, taxanes, and other taxol-like drugs, e.g., Taxotere, and the like), camptothecin and derivatives thereof; aspirin; ibuprofen; piroxicam; cimetidine; substantially water insoluble steroids such as estrogen, prednisolone, cortisone, hydrocortisone, diflorasone, and the like; drugs such as phenesterine, duanorubicin, doxorubicin, mitotane, visadine, halonitrosoureas, anthrocylines, ellipticine, diazepam, and the like; and anaesthetics such as methoxyfluorane, isofluorane, enfluorane, halothane, benzocaine, dantrolene, barbiturates, combinations thereof, and the like. Additional useful active agents include substantially water insoluble immunosuppressive agents, such as, for example but not by limitation, cyclosporines, azathioprine, FK506, prednisone, combinations thereof, and the like.

According to further embodiments, pharmaceutical and/or biologic compounds and/or compositions useful as active agents in the particles of the present invention can be found in and throughout the references and patent applications incorporated herein by reference.

Where the active agent is added in addition to a carrier or active protein, the active agent will comprise from about 0.5 to about 20, about 30, about 40%, about 50%, about 60%, about 70% by weight (w/w) of the nanoparticle. In this manner, the nanoparticles can be formulated into pharmaceutical compositions to deliver an effective amount of the active agent. By “effective amount” of an active agent is the amount necessary to elicit the desired biological response. The effective amount of an agent may vary depending on such factors as the desired biological endpoint, the agent to be delivered, the composition of the carrier protein where a carrier protein is used, the target cells, etc. For example, the effective amount of nanoparticles containing an antigen to be delivered to immunize an individual is the amount that results in an immune response sufficient to prevent infection with an organism having the administered antigen.

iii) Modified Polymers/Proteins

According to some embodiments of the present invention, the organic or polymer component of the pharmaceutical organic particle can be cross-linked either during formation of the particle or after formation of the particle such that the pharmaceutical organic particle is engineered to degrade in a selected environment, after a predetermined amount of time, to a predetermined chemical or physical stimuli, combinations thereof, or the like. In one embodiment, the polymer component of the particle is chemically cross-linked through the formation of disulfide bonds through, for example, the amino acid cysteine that occurs in the natural structure of a number of proteins.

In some embodiments, the polymer component of the particle can be modified or configured to properties for particular applications and/or environments. These biocompatible materials can, in some embodiments, be cross-linked or uncross-linked to provide matrices from which a pharmacologically active ingredient, for example a taxane, other chemotherapeutic or therapeutic agents, antiinfective, or immunosuppressive agents can be released by diffusion and/or degradation of the matrix in selected environments. In other embodiments, temperature sensitive materials can be used with the particles of the present invention to change physical form and release a pharmacologically active cargo in a desired environment. An example of temperature sensitive materials for use in the particles of the present invention include, but are not limited to, copolymers of polyacrylamides or copolymers of polyalkylene glycols and polylactide/glycolides, combinations thereof, and the like.

According to some embodiments, the polymer can be configured through covalent bonding with an agent. Covalent bonds operable with the present invention include, but are not limited to, ester, ether, urethane, diester, amide, primary, secondary or tertiary amine, phosphate ester, sulfate ester, thiol, carborxylic acid, amino acid, combination thereof, and the like bonds. Suitable agents contemplated for this optional modification of the polymeric shell include synthetic polymers (polyalkylene glycols (e.g., linear or branched chain polyethylene glycol), polyvinyl alcohol, polyhydroxyethyl methacrylate, polyacrylic acid, polyethyloxazoline, polyacrylamide, polyvinyl pyrrolidinone, and the like), phospholipids (such as phosphatidyl choline (PC), phosphatidyl ethanolamine (PE), phosphatidyl inositol (PI), sphingomyelin, and the like), proteins (such as enzymes, antibodies, and the like), polysaccharides (such as starch, cellulose, dextrans, alginates, chitosan, pectin, hyaluronic acid, and the like), chemical modifying agents (such as pyridoxal 5′-phosphate, derivatives of pyridoxal, dialdehydes, diaspirin esters, and the like), combinations thereof, or the like.

According to still further embodiments, albumin particles fabricated according to methods disclosed herein can be configured or modified, post fabrication, for targeting specific cells, organs, tumors, and other human and animal tissue including, but not limited to, avidin/biotin complex, MAbs, targeting peptides, and aptamers. In other embodiments, particle surfaces can be configured such as by but not limited to, reacting primary amines, alcohols, carboxylic acids, thiols, or other moieties contained in albumin with CDI or the like for further modification with any nucleophile or electrophile.

According to some embodiments, after molding and formation of protein particles using the methods described herein and harvesting (described elsewhere herein) the particles can be modified to produce predetermined degradable particles. The predetermined degradable particles are formed to degrade under specific environmental, chemical, time, physical, or other stimuli but remain otherwise substantially intact. Therefore, the particles can be tailored to survive in biological, or other environments, such that the active or pharmaceutical agent remains intact until the particle reaches a desired location or environmental condition. In some embodiments, the particle is harvested into a solvent where the particles remain intact and the individual protein molecules of the particle can be modified to form predetermined degradable particles. In some embodiments, the particles can be harvested onto a film or backing and modified while adhered to the film to form the predetermined degradable particles.

In some embodiments, the particles are modified by chemically crosslinking the polymer or proteins of the particle. In some embodiments, the proteins can be chemically crosslinked either through the nitrogen functionality of N-terminus or lysine residues or the thiol functionality of cysteine residues using an appropriate difunctional crosslinker, e.g. glutaraldehyde or bis-maleimidohexane. In other embodiments, the crosslinkers can also have a functional group between the two protein reactive groups that can degrade under specific in vivo conditions, such as for example, disulfide, acetal, ketal, or hydrazone, as shown in FIG. 11. According to some embodiments, the surface crosslinking of the particles with degradable crosslinks can allow an entrapped cargo to remain within the particle and maintain particle size and shape until the particle is endocytosed by a cell. Once the particle has entered the cell an appropriate intracellular trigger, which can be predetermined when fabricating the particle, will cause the particle to degrade and release entrapped cargo.

In further embodiments, as opposed to crosslinking particles with difunctional crosslinking compounds after particle formation, the particles can be crosslinked without using an external crosslinking reagent because albumin, and other proteins, have cysteine residues or that proteins such as albumin tends to aggregate at elevated temperatures. The formation of disulfide bonds between cysteine residues of different albumin molecules can, in some embodiments, be induced by the application of mechanical force (e.g. sonication) or treating the particles with a gentle oxidizing agent (e.g. iodine). Human albumin has multiple cysteine residues for crosslinking and the intermolecular disulfide bonds can provide sufficient crosslinking for the particle to retain its size, shape, and cargo. For example, the albumin particles can be harvested into a non-solvent such as chloroform and then be exposed to ultrasonication for a period of time to induce the formation of intermolecular disulfide bonds. Similarly, albumin particles in chloroform could be treated with a chloroform solution of iodine to induce disulfide formation. Also, taking albumin particles and treating them with mild heating (i.e. 65-70 degrees C.) to induce aggregation of the albumin molecules within the particle prior to resuspending in an aqueous environment can impart further stability to the pharmaceutical organic particle of the present invention.

According to some embodiments of the present invention, certain pharmaceutical organic particle formulations disclosed herein are useful for the treatment of a variety of indications, including for example but not limitation, brain tumors, intraperitoneal tumors, prostatitis, bph, restenosis, atherosclerosis, cancers of prostate, testes, lung, kidney, pancreas, bone, spleen, liver, brain, combinations thereof, and the like.

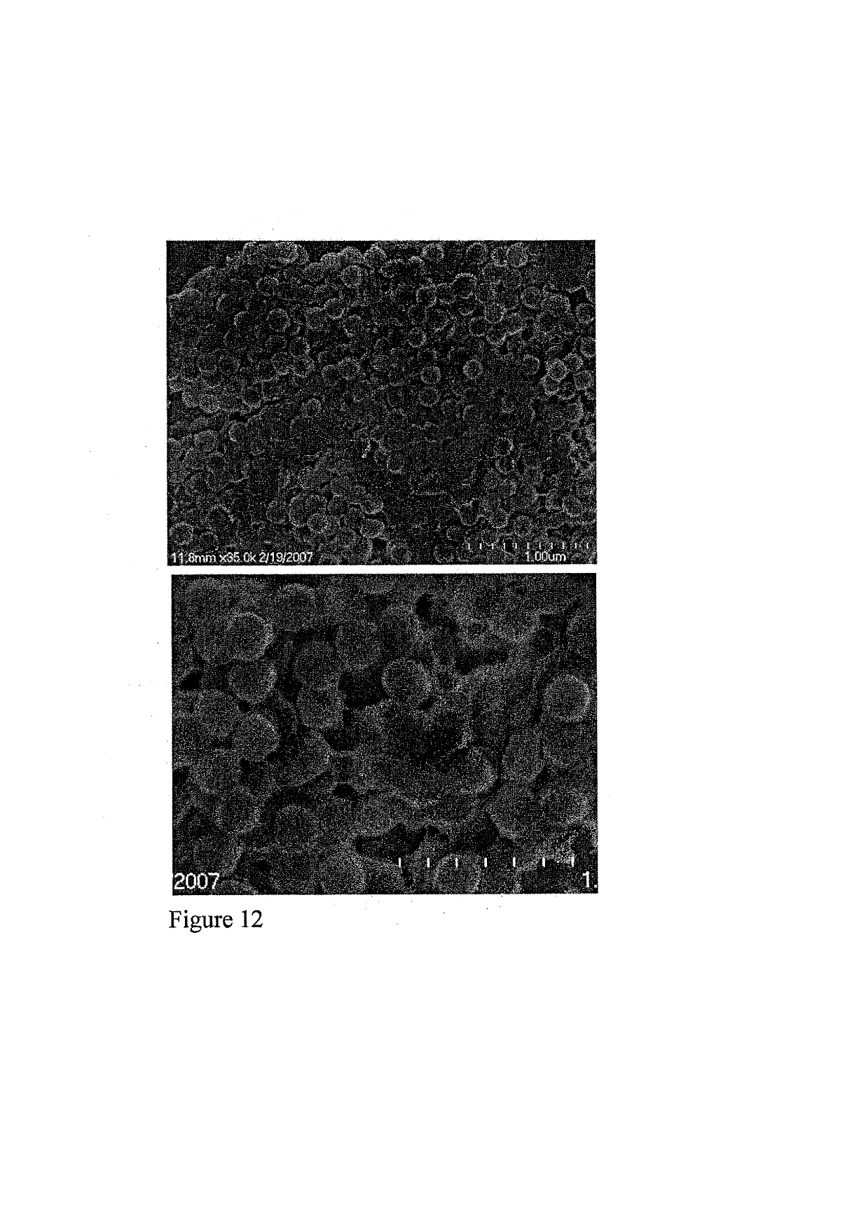

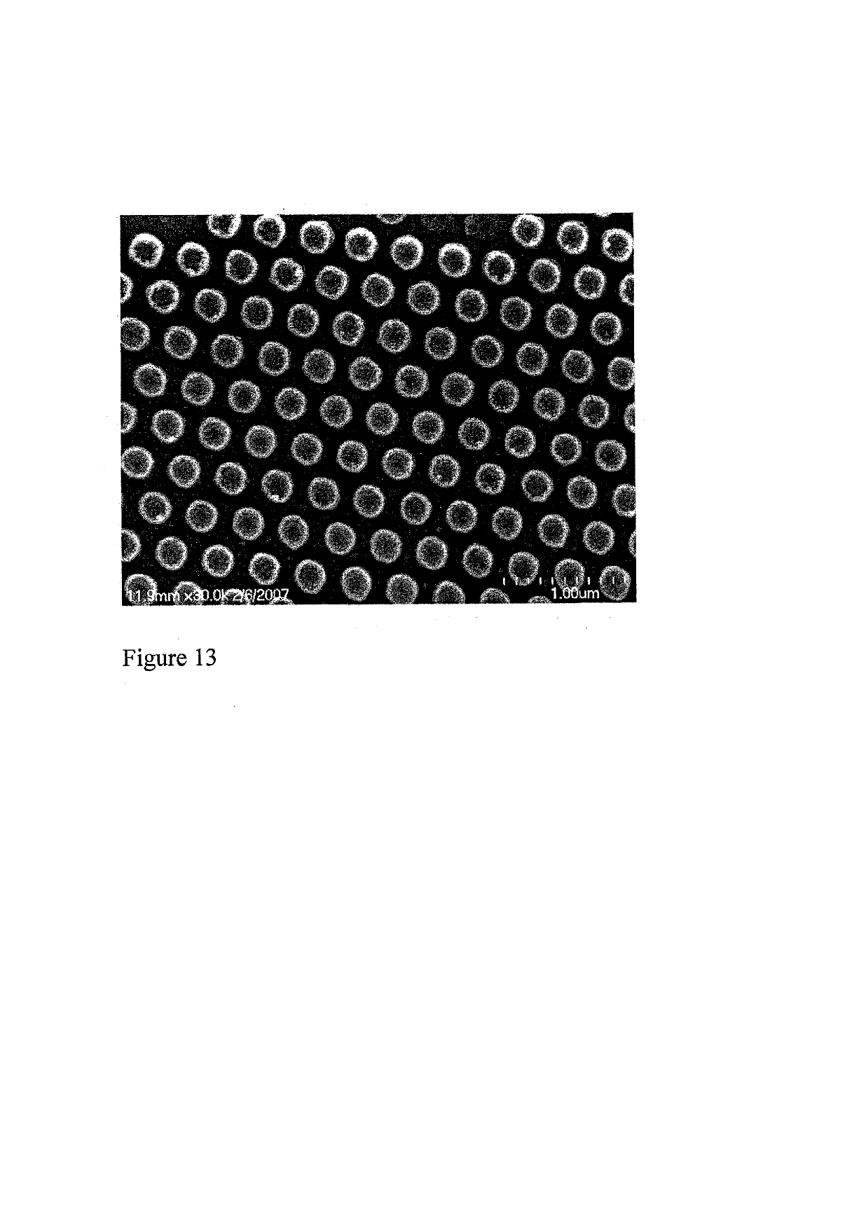

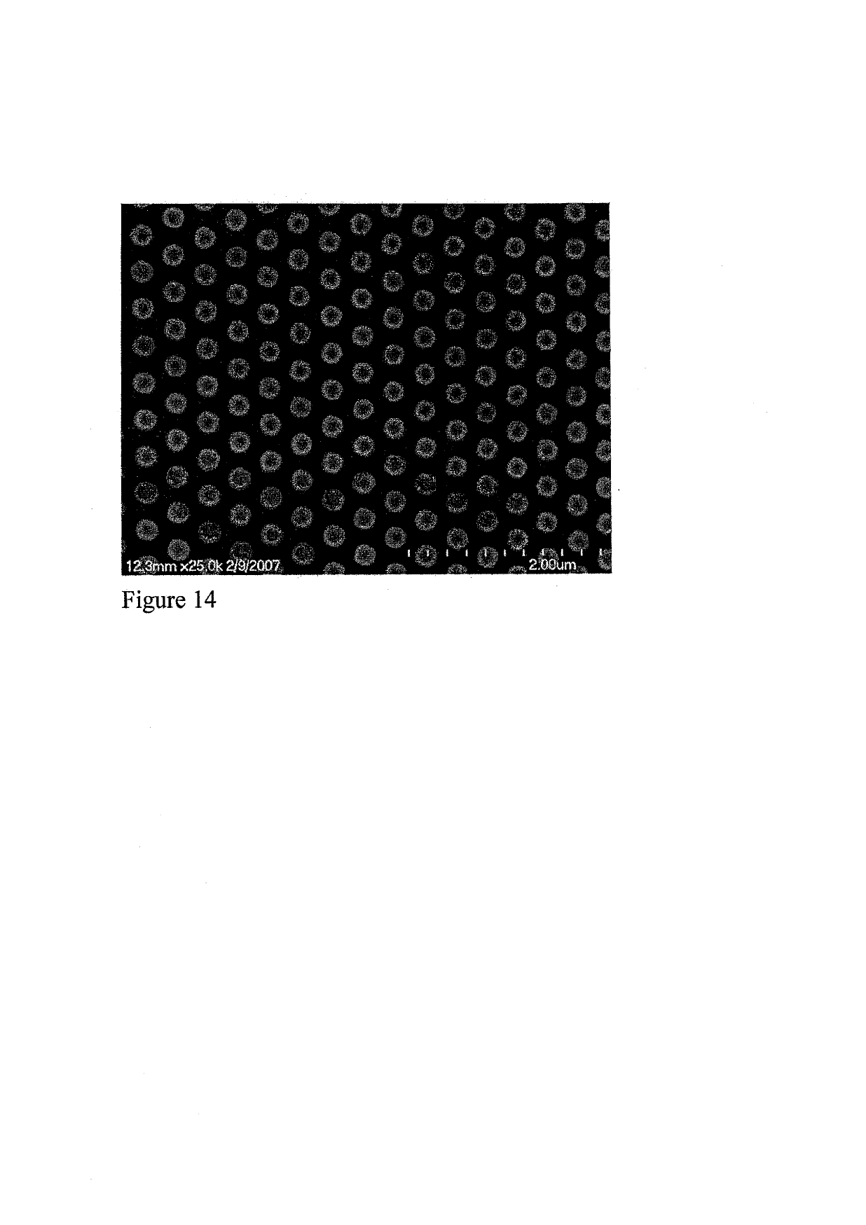

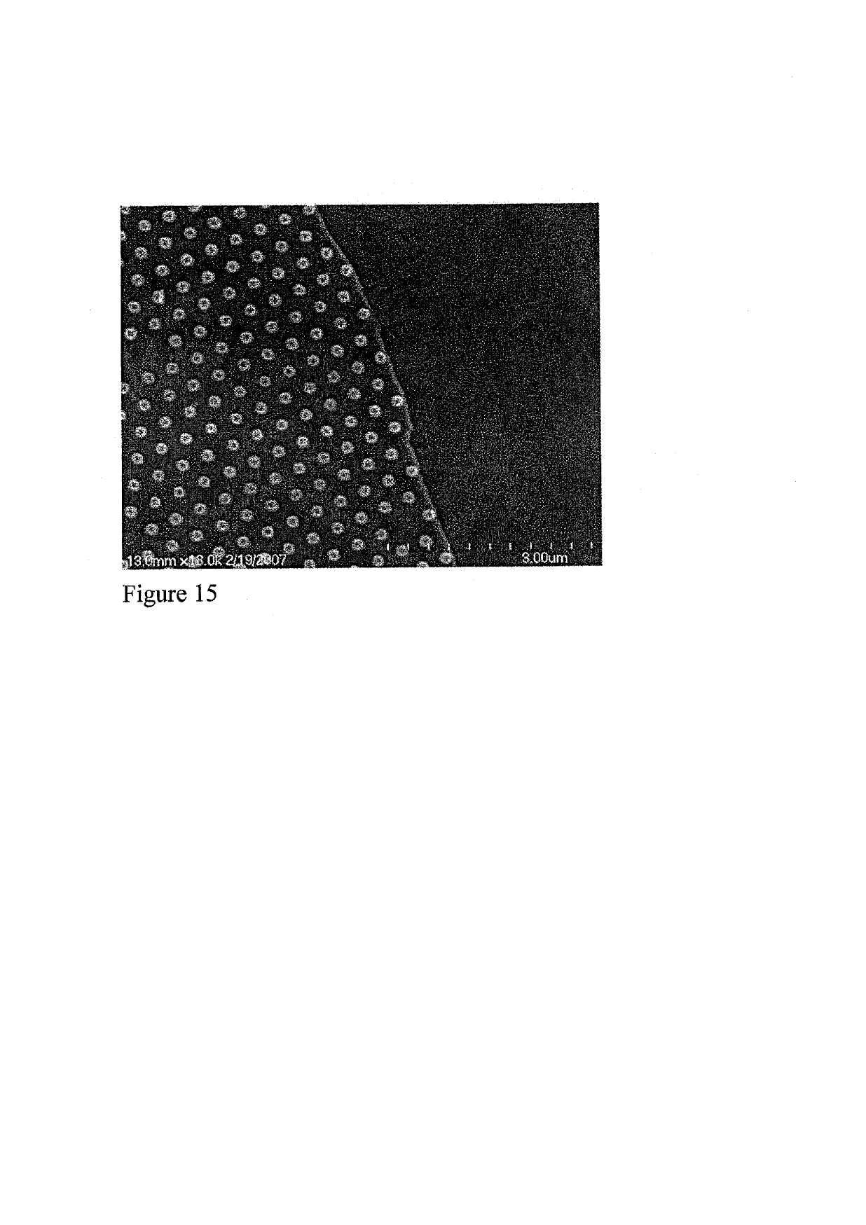

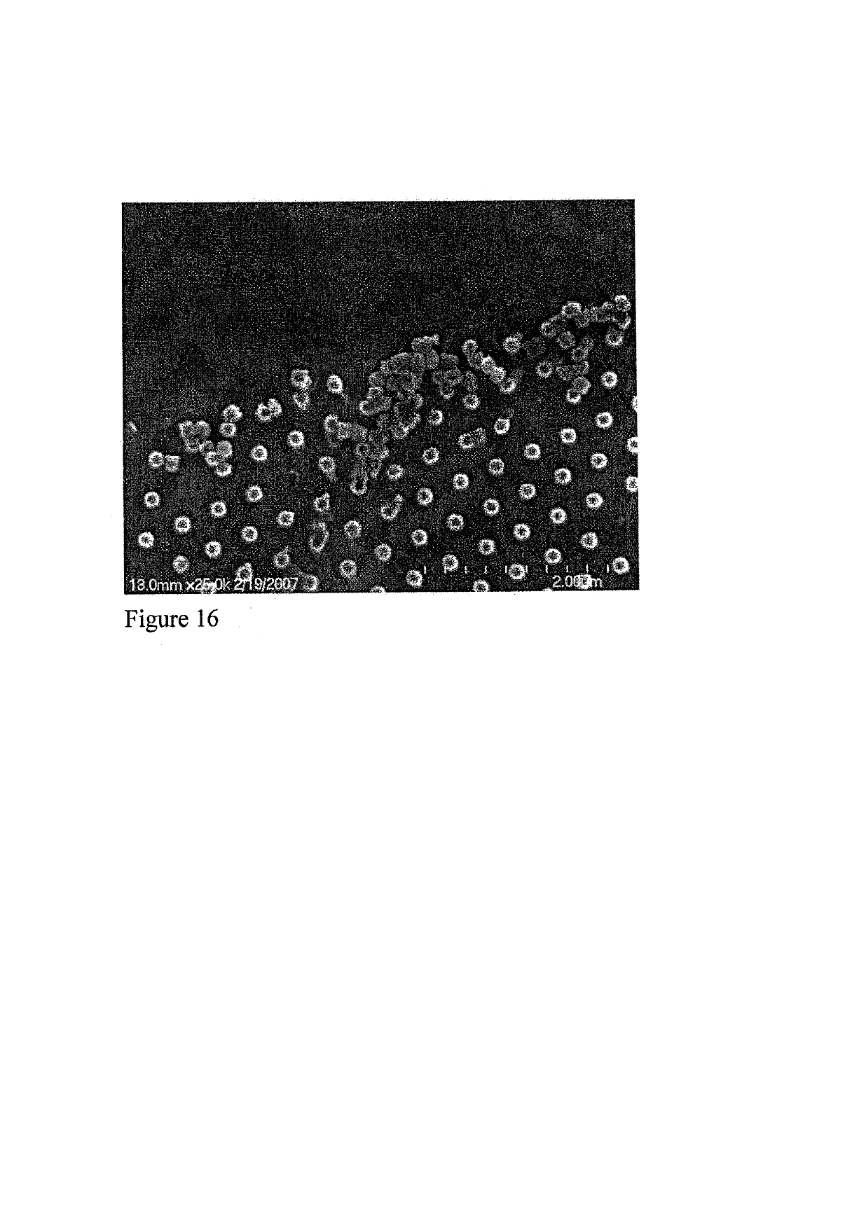

According to an embodiment of the present invention, human serum albumin particles were molded in 200 nm tall×200 nm diameter cylinder cavities of the molds described herein to form human serum albumin particles substantially 200 nm tall×200 nm in diameter, as shown in FIG. 12. In another embodiment, human serum albumin was added to deionized water and ethyleneglycol and introduced to 200 nm tall×200 nm diameter cylinder cavities to form particles substantially 200 nm tall×200 nm in diameter, as shown in FIG. 13. According to another embodiment, siRNA was added an albumin/PBS solution and molded into 200 nm tall×200 nm diameter cylinder particles according to the methods and materials described herein, as shown in FIG. 14. In another embodiment, particles were fabricated of albumin, EDC in water, water, and ethylene glycol were fabricated in 200 nm tall×200 nm diameter cylinders according to the methods and materials described herein and as shown in FIG. 15. In yet another embodiment, particles of human transferrin and a phosphate buffered saline were fabricated in 200 nm tall×200 nm diameter cylinders according to the methods and materials described herein and as shown in FIG. 16.

According to other embodiments, nano-particles of virtually any shape can be formed according to the methods described herein. In some embodiments, the nano-particles can have compositions such as, for example but not as a limitation: paclitaxel and human serum albumin; human albumin serum particles treated with a water insoluble cross-linker, such as dithiobis[succinimidylpropionate] (DSP) or disuccinimidyl suberate (DSS); human albumin serum particles treated with a water soluble crosslinker, such as 3,3″-Dithiobis(sulfosuccinimidylpropionate) (DTSSP); albumin combined with hydrophilic and/or hydrophobic agents including, but not limited to, siRNA, paclitaxel, doxorubicin, Rapamyacin, Sirolimus, antisense oligonucleotides, enzymes, protease, combinations thereof, and the like.

VI. Harvesting

Before using the pharmaceutical organic particles formed in the cavities of the patterned templates, the particles must, in most embodiments, be removed from the cavities. This can be accomplished by a number of approaches, including but not limited to applying the patterned template containing the particles in the cavities to a surface that has an affinity for the particles that is greater than an affinity between the particles and the cavities of the patterned template; applying the patterned template containing the particles to a material that, when hardened, has a chemical and/or physical interaction with the particles and binds the particles; deforming the patterned template such that the particle is released from the patterned template; swelling the patterned template with a first solvent to extrude the particles; or washing the patterned template with a second solvent that has an affinity for the particles.

In some embodiments, the surface that has an affinity for the particles includes an adhesive or sticky surface (e.g. carbohydrates, epoxies, waxes, polyvinyl alcohol, polyvinyl pyrrolidone, polybutyl acrylate, polycyano acrylates, polymethyl methacrylate). In some embodiments, the liquid is water that is cooled to form ice while in contact with the particles. In some embodiments, the water is cooled to a temperature below the Tm of water but above the Tg of the particle. In some embodiments the water is cooled to a temperature below the Tg of the particles but above the Tg of the mold or substrate. In some embodiments, the water is cooled to a temperature below the Tg of the mold or substrate.

In some embodiments, the first solvent includes supercritical fluid carbon dioxide. In some embodiments, the first solvent includes water. In some embodiments, the first solvent includes an aqueous solution including water and a detergent. In embodiments, the deforming the surface element is performed by applying a mechanical force to the surface element. In some embodiments, the method of removing the patterned structure further includes a sonication method.

In some embodiments, the harvesting methods include a process selected from the group including scraping with a doctor blade, a brushing process, a dissolution process, an ultrasound process, a megasonics process, an electrostatic process, and a magnetic process. In some embodiments, the harvesting or collecting of the particles includes applying a material to at least a portion of a surface of the particle wherein the material has an affinity for the particles. In some embodiments, the material includes an adhesive or sticky surface. In some embodiments, the material includes, without limitation, one or more of a carbohydrate, an epoxy, a wax, polyvinyl alcohol, polyvinyl pyrrolidone, polybutyl acrylate, a polycyano acrylate, a polyacrylic acid and polymethyl methacrylate. In some embodiments, the harvesting or collecting of the particles includes cooling water to form ice (e.g., in contact with the particles). In some embodiments, the presently disclosed subject matter describes a particle or plurality of particles formed by the methods described herein. In some embodiments, the plurality of particles includes a plurality of monodisperse particles. In some embodiments, the particle or plurality of particles provide gaps between components of a device, such devices being selected from the group including, but not limited to a semiconductor device, a photovoltaic device, an additive, a sensor, an abrasive, a micro-electro-mechanical system (MEMS), an optical device, an electronic device, an automotive device, and the like.

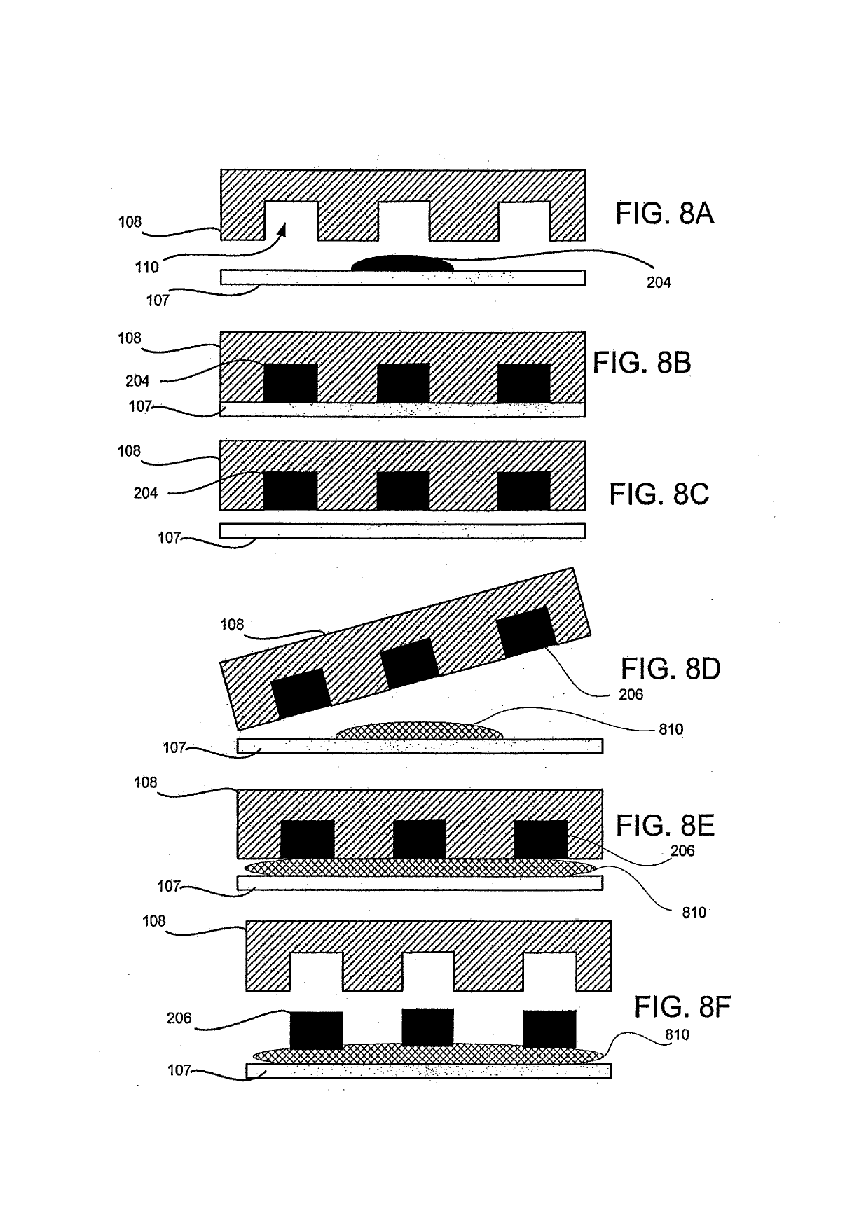

Further embodiments of particle harvesting methods are shown in FIGS. 8A-8F. According to FIGS. 8A-8F, particles 206 are formed in patterned template cavities 110 according to other embodiments described herein and in references cited and incorporated herein by reference. After particles 206 are fabricated in cavities 110 of patterned template 108, particles 206 are contacted with harvesting material 810, as shown in FIG. 8D. As the combination of patterned template 108 and particle 206 comes into contact with harvesting material 810, harvesting material 810 is distributed across patterned template 108 and contacts the plurality of particles 206 by being pinched between patterned template 108 and backing 107, as shown in FIG. 8E. In some embodiments, harvesting material 108 has a greater affinity for particles 206 than an affinity between particles 206 and patterned template 108, thereby; particles 206 remain in contact with harvesting material 810 when patterned template 108 is removed, as shown in FIG. 8F. According to some embodiments, harvesting material 810 can be an adhesive, a liquid, water, a monomer, a polymer, a biodegradable substance, combinations thereof, or the like. In some embodiments, harvesting material 810 can be solidified, hardened, or cured to form a harvesting film to which particles 206 become affixed or removably affixed. In some embodiments, the particles are removably affixed to a harvesting film by applying cyanoacrylate between the film and particles 206. Particles 206 can be utilized on the film, modified while on the film, packaged on the film, applied to an end use while on the film, or removed from the film by dissolving the affixing substance or overcoming the affinity between the film and the particles 206.

In one embodiment harvesting material 810 has an affinity for particles 206. For example, in some embodiments, harvesting material 810 includes an adhesive or sticky surface. In other embodiments, harvesting material 810 undergoes a transformation after it is brought into contact with particles 206. In some embodiments that transformation is an inherent characteristic of harvesting material 810. In other embodiments, harvesting material 810 is treated to induce the transformation. For example, in one embodiment harvesting material 810 is an epoxy that hardens after it is brought into contact with particles 206. Thus when harvesting material 810 is pealed away from backing 107, particles 206 remain engaged with the epoxy and not backing 107. In other embodiments, harvesting material 810 is water that is cooled to form ice. Thus, when backing 107 is stripped from the ice, particles 206 remain in communication with the ice and not backing 107. In one embodiment, the particle-containing ice can be melted to create a liquid with a concentration of particles 206. In some embodiments, harvesting material 810 include, without limitation, one or more of a carbohydrate, an excipient, an epoxy, a wax, polyvinyl alcohol, polyvinyl pyrrolidone, polybutyl acrylate, a polycyano acrylate and polymethyl methacrylate. In some embodiments, harvesting material 810 includes, without limitation, one or more of liquids, solutions, powders, granulated materials, semi-solid materials, suspensions, combinations thereof, or the like.

According to yet another embodiment the particles are harvested on a fast dissolving substrate, sheet, or films. The film-forming agents can include, but are not limited to pullulan, hydroxypropylmethyl cellulose, hydroxyethyl cellulose, hydroxypropyl cellulose, polyvinyl pyrrolidone, carboxymethyl cellulose, polyvinyl alcohol, sodium alginate, polyethylene glycol, xanthan gum, tragacanth gum, guar gum, acacia gum, arabic gum, polyacrylic acid, methylmethacrylate copolymer, carboxyvinyl polymer, amylose, high amylose starch, hydroxypropylated high amylose starch, dextrin, pectin, chitin, chitosan, levan, elsinan, collagen, gelatin, zein, gluten, soy protein isolate, whey protein isolate, casein, combinations thereof, and the like. In some embodiments, pullulan is used as the primary filler. In still other embodiments, pullulan is included in amounts ranging from about 0.01 to about 99 wt %, preferably about 30 to about 80 wt %, more preferably from about 45 to about 70 wt %, and even more preferably from about 60 to about 65 wt % of the film. In some embodiments, the particles are harvested on a GRAS (“generally recognized as safe”) material. A list of current GRAS materials can be found in 21 C.F.R. Part 182; 21 C.F.R. Part 184, and 21 C.F.R. Part 186 as well as the Food and Drug Administration web site, each of which are incorporated herein by reference.

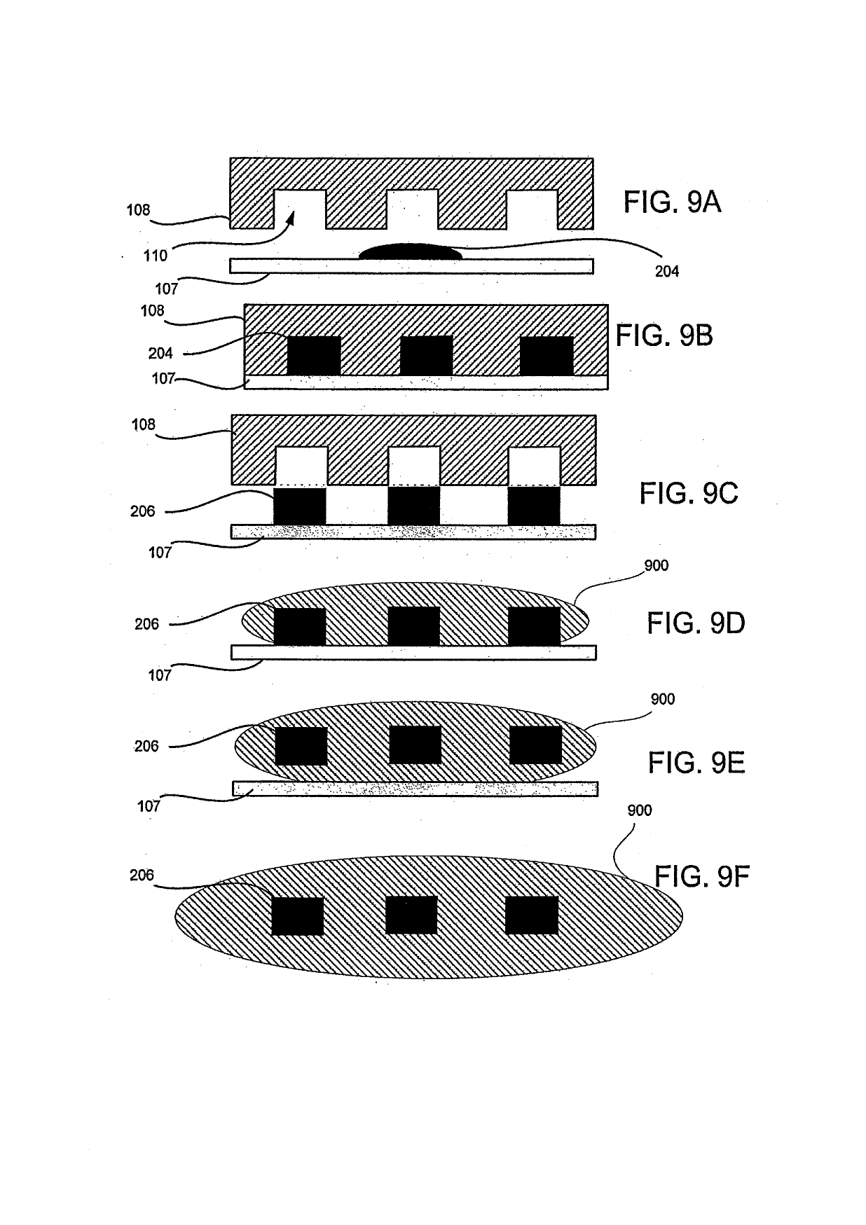

Referring now to FIGS. 9A-9F, particles 206 are formed in patterned template 108 cavities 110 according to other embodiments described herein and in references cited and incorporated herein by reference. After particles 206 are fabricated in cavities 110 of patterned template 108 (FIGS. 9A-9C), harvesting solution 900 is introduced to particles 206, as shown in FIG. 9D. In some embodiments, harvesting solution 900 can be any composition into which particles 206 can go into solution with or disassociate from backing 107, such as shown in FIGS. 9E and 9F. It will be appreciated that depending on the composition of particles 206, the composition of harvesting solution 900 will vary; however, such selection is within the understanding of one skilled in the art. Thereafter, particles 206, being in solution with harvesting solution 900 can be utilized and introduced to a predetermined application.

According to other embodiments, particles 206 are harvested by subjecting the particle/cavity combination to a physical force or energy such that particles 206 are released from the cavities 110. In some embodiments the force is, but is not limited to, centrifugation, dissolution, vibration, ultrasonics, megasonics, gravity, flexure of the template, suction, electrostatic attraction, electrostatic repulsion, magnetism, physical template manipulation, combinations thereof, and the like.

According to some embodiments, particles 206 are purified after being harvested. In some embodiments particles 206 are purified from the harvesting substance. In some embodiments, the particles 206 are modified or functionalized, as described herein, before they are harvested from the cavities. In some embodiments, the particles 206 are modified or functionalized, as described herein, after they are harvested from the cavities. The harvesting can be, but is not limited to, centrifugation, separation, vibration, gravity, dialysis, filtering, sieving, electrophoresis, gas stream, magnetism, electrostatic separation, combinations thereof, and the like.

According to some embodiments, the harvesting substance is, but is not limited to, water, organic solvents, carbohydrates, epoxies, waxes, polyvinyl alcohol, polyvinyl pyrrolidone, polybutyl acrylate, polycyano acrylates, polymethyl methacrylate, a porogen, combinations thereof, and the like.

VII. Formulations and Administration

The protein micro and/or nanoparticles described herein may be present in a dry formulation or suspended in a biocompatible medium. Suitable biocompatible media include, but are not limited to, water, buffered aqueous media, saline, buffered saline, optionally buffered solutions of amino acids, optionally buffered solutions of proteins, optionally buffered solutions of sugars, optionally buffered solutions of vitamins, optionally buffered solutions of synthetic polymers, lipid-containing emulsions, and the like.

The pharmaceutical composition of the invention can include other agents, excipients, or stabilizers. For example, to increase stability by increasing the negative zeta potential of nanoparticles, certain negatively charged components may be added. Such negatively charged components include, but are not limited to bile salts of bile acids consisting of glycocholic acid, cholic acid, chenodeoxycholic acid, taurocholic acid, glycochenodeoxycholic acid, taurochenodeoxycholic acid, litocholic acid, ursodeoxycholic acid, dehydrocholic acid and others; phospholipids including lecithin (egg yolk) based phospholipids which include the following phosphatidylcholines: palmitoyloleoylphosphatidylcholine, palmitoyllinoleoylphosphatidylcholine, stearoyllinoleoylphosphatidylcholine stearoyloleoylphosphatidylcholine, stearoylarachidoylphosphatidylcholine, and dipalmitoylphosphatidylcholine. Other phospholipids including L-.alpha.-dimyristoylphosphatidylcholine (DMPC), dioleoylphosphatidylcholine (DOPC), distearoylphosphatidylcholine (DSPC), hydrogenated soy phosphatidylcholine (HSPC), and other related compounds. Negatively charged surfactants or emulsifiers are also suitable as additives, e.g., sodium cholesteryl sulfate and the like.

Formulations suitable for oral administration can consist of (a) liquid solutions, such as an effective amount of the compound dissolved in diluents, such as water, saline, or orange juice, (b) capsules, sachets or tablets, each containing a predetermined amount of the active ingredient, as solids or granules, (c) suspensions in an appropriate liquid, and (d) suitable emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such excipients as are known in the art.

Examples of suitable carriers, excipients, and diluents include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline solution, syrup, methylcellulose, methyl- and propylhydroxybenzoates, talc, magnesium stearate, and mineral oil. The formulations can additionally include lubricating agents, wetting agents, emulsifying and suspending agents, preserving agents, sweetening agents or flavoring agents.

Formulations suitable for parenteral administration include aqueous and non-aqueous, isotonic sterile injection solutions, which can contain anti-oxidants, buffers, bacteriostats, and solutes that render the formulation compatible with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

In some embodiments, the composition is formulated to have a pH range of about 4.5 to about 9.0, including for example pH ranges of any of about 5.0 to about 8.0, about 6.5 to about 7.5, and about 6.5 to about 7.0. In some embodiments, the pH of the composition is formulated to no less than about 6, including for example no less than about any of 6.5, 7, or 8 (such as about 8). The composition can also be made to be isotonic with blood by the addition of a suitable tonicity modifier, such as glycerol.

The pharmaceutical compositions comprising the protein micro and/or nanoparticles described herein can be administered to an individual (such as human) via various routes, such as parenterally, including intravenous, intra-arterial, intraperitoneal, intrapulmonary, oral, inhalation, intravesicular, intramuscular, intra-tracheal, subcutaneous, intraocular, intrathecal, or transdermal. For example, the nanoparticle composition can be administered by inhalation to treat conditions of the respiratory tract. The composition can be used to treat respiratory conditions such as pulmonary fibrosis, bronchiolitis obliterans, lung cancer, bronchioalveolar carcinoma, and the like. In some embodiments, the nanoparticle composition is administrated intravenously. In some embodiments, the nanoparticle composition is administered orally.

The pharmaceutical compositions of the invention provide for better biodistribution of the active agent upon administration. Additionally, the particles allow for enhanced stability. In this manner, more of the active agent is delivered at the target site.

### EXAMPLES

### Example 1.1

### Preparation of Albumin PRINT Particles with Direct Harvesting

A patterned perfluoropolyether (PFPE) mold was generated by pouring a PFPE-dimethacrylate (PFPE-DMA) containing 1-hydroxycyclohexyl phenyl ketone over a silicon substrate patterned with 200 nm tall×200 nm diameter cylinders. The PFPE-DMA covered master was then subjected to UV light (λ=365 nm) for 3 minutes while under a nitrogen purge. The fully cured PFPE-DMA mold was then released from the silicon master. Separately, human serum albumin was added to 1:1 v/v deionized water/ethyleneglycol solution to make a 35 weight % composition. This mixture was spotted directly onto the contact point of the patterned PFPE-DMA mold and an unpatterned polyethyleneterethalate film affixed on a laminator. The stage of the laminator moved at a speed of 2.0 with 50 psi pressure put onto the roller. The solution filled mold was then placed in a protective area on the bench top overnight for slow solvent evaporation. The particles were harvested by placing 2 mL of chloroform or DMSO on the mold and scraping the surface with a glass slide. The particle suspension was transferred to a scintillation vial. Roughly 10 μL of this solution was spotted onto a glass slide dried under vacuum and is shown in FIG. 12.

### Example 1.2

### Preparation of Albumin PRINT™ Particles with Use of a Medical Adhesive Layer for Harvesting

A patterned perfluoropolyether (PFPE) mold was generated by pouring a PFPE-dimethacrylate (PFPE-DMA) containing 1-hydroxycyclohexyl phenyl ketone over a silicon substrate patterned with 200 nm tall×200 nm diameter cylinders. The PFPE-DMA covered master was then subjected to UV light (λ=365 nm) for 3 minutes while under a nitrogen purge. The fully cured PFPE-DMA mold was then released from the silicon master. Separately, 58 mg human serum albumin was added to 54 μL of deionized water as well as 54 μL ethyleneglycol. This mixture was spotted directly onto the contact point of the patterned PFPE-DMA mold and an unpatterned polyethyleneterethalate film affixed on a laminator. The stage of the laminator moved at a speed of 2.0 with 50 psi pressure put onto the roller. The solution filled mold was then placed in a protective area on the bench top overnight for slow solvent evaporation. After overnight evaporation, the filled molds were placed on a glass slide spotted with cyanoacrylate and the medical was allowed to polymerize. Subsequently, the mold was peeled off the adhesive layer. The particles are thus transferred to the medical adhesive and shown in FIG. 13.

### Example 1.3

### Preparation of siRNA Encapsulated Albumin PRINT Particles

A patterned perfluoropolyether (PFPE) mold was generated by pouring a PFPE-dimethacrylate (PFPE-DMA) containing 1-hydroxycyclohexyl phenyl ketone over a silicon substrate patterned with 200 nm tall×200 nm diameter cylinders. The PFPE-DMA covered master was then subjected to UV light (λ=365 nm) for 3 minutes while under a nitrogen purge. The fully cured PFPE-DMA mold was then released from the silicon master. Separately, 4 mg of human serum albumin was added to 4 μL sterile PBS. A 20 μL solution of siRNA was acquired (Anti-Luciferase siRNA from Dharmacon, Part number D-001400-01). This is a double stranded RNA molecule with 21-base pairs in each strand, with the following sequence:

5′-CUUACGCUGAGUACUUCGATT
TTGAAUGCGACUCAUGAAGCU

The 20 μL solution of siRNA in water at [1 μg/μL] was lyophilized overnight. To dry siRNA (20 μg) was added 4 μL of albumin/PBS solution. This mixture was spotted directly onto the contact point of the patterned PFPE-DMA mold and an unpatterned polyethyleneterethalate film affixed on a laminator. The stage of the laminator moved at a speed of 2.0 with 30 psi pressure put onto the roller. The solution filled mold was then placed in a protective area on the bench top overnight for slow solvent evaporation. After overnight evaporation, the filled molds were placed on a glass slide spotted with cyanoacrylate and the medical adhesive was allowed to polymerize. Subsequently, the mold was peeled off the adhesive layer. The particles are thus transferred to the medical adhesive and shown in FIG. 14.

### Example 1.4

### Modified albumin PRINT particles using 1-ethyl-3-[3-dimethylaminopropyl]carbodiimide hydrochloride (EDC)

A patterned perfluoropolyether (PFPE) mold was generated by pouring a PFPE-dimethacrylate (PFPE-DMA) containing 1-hydroxycyclohexyl phenyl ketone over a silicon substrate patterned with 200 nm tall×200 nm diameter cylinders. The PFPE-DMA covered master was then subjected to UV light (λ=365 nm) for 3 minutes while under a nitrogen purge. The fully cured PFPE-DMA mold was then released from the silicon master. Separately, to a formulation consisting of 58 mg albumin, was added 45 μL of 2 mg/mL EDC in water, 9 μL water and 54 μL ethylene glycol. This mixture was spotted directly onto the contact point of the patterned PFPE-DMA mold and an unpatterned polyethyleneterethalate film affixed on a laminator. The stage of the laminator moved at a speed of 2.0 with 30 psi pressure put onto the roller. The solution filled mold was then placed in a protective area on the bench top overnight for slow solvent evaporation and allowed for crosslinking to proceed. Particles were observed in the mold cavities.

Another formulation was composed of 58 mg albumin, 52 μL water, 54 μL ethyleneglycol, and 2 μL of 2 mg/mL EDC. This mixture was spotted directly onto the contact point of the patterned PFPE-DMA mold and an unpatterned polyethyleneterethalate film affixed on a laminator. The stage of the laminator moved at a speed of 2.0 with 30 psi pressure put onto the roller. The solution filled mold was then placed in a protective area on the bench top overnight for slow solvent evaporation and allowed for crosslinking to proceed. Particles were observed in the mold cavities.

### Example 1.5

### Harvesting Albumin PRINT Particles Using Lyophilization

A filled mold of varying albumin compositions in varying solvent systems, for example 58 mg albumin in 54 μL, water and 54 μL, ethylene glycol, was placed in the freezer for 4 hours or until frozen and quickly placed in a lyophilizing chamber and attached to a lyophilizer (LABCONCO, FreeZone 4.5) overnight of until all solvent has been removed. The filled mold was then removed from the lyophilizer and chamber for harvesting. Direct harvesting using chloroform and the sipper method was employed.

### Example 1.6

### Preparation of Albumin Particles from Water and Direct Harvesting

A patterned perfluoropolyether (PFPE) mold was generated by pouring a PFPE-dimethacrylate (PFPE-DMA) containing 1-hydroxycyclohexyl phenyl ketone over a silicon substrate patterned with 200 nm tall×200 nm diameter cylinders. The PFPE-DMA covered master was then subjected to UV light (λ=365 nm) for 3 minutes while under a nitrogen purge. The fully cured PFPE-DMA mold was then released from the silicon master. Separately, human serum albumin was added to deionized water to make a 35 weight % composition. This mixture was spotted directly onto the contact point of the patterned PFPE-DMA mold and an unpatterned polyethyleneterethalate film affixed on a laminator. The stage of the laminator moved at a speed of 2.0 with 50 psi pressure put onto the roller. The solution filled mold was then placed in the freezer for at least four hours and subsequently lyophilized overnight. To the lyophilized filled mold was added 1 mL chloroform and with gentle mechanical force using a glass slide, the particles were extracted from the mold and harvested. Chloroform was slowly evaporated at ambient conditions to afford dry particles.

### Example 1.7

### Preparation of Albumin Particles from Water and Harvesting on Medical Adhesive

A drop of cyanoacrylate was added to a substrate (glass or untreated PET) and a filled, lyophilized mold was placed atop the cyanoacrylate drop, pattern down, and rolled out such that the drop underneath the mold spread. Once the cyanoacrylate was polymerized, the mold was lifted, yielding particles harvested onto the adhesive layer. Individual particles are obtainable by dissolving the adhesive layer with acetone.

### Example 1.8

### Preparation of Albumin Particles from Water and Harvesting on Excipient (Povidone)

A drop of 10 wt % poly(vinylpyrrolidone) (PVP) in water was added to a substrate (glass or untreated PET) and spread onto the substraight using a Meyer Rod. The filled, lyophilized mold was placed atop the PVP solution, pattern down, and rolled out onto the spread PVP solution. Once the PVP had dried, the mold was lifted, yielding particles harvested onto the excipient (PVP) layer.

### Example 1.9

### Preparation of Albumin with an Excipient (Trehalose) in the Composition

A patterned perfluoropolyether (PFPE) mold was generated by pouring a PFPE-dimethacrylate (PFPE-DMA) containing 1-hydroxycyclohexyl phenyl ketone over a silicon substrate patterned with 200 nm tall×200 nm diameter cylinders. The PFPE-DMA covered master was then subjected to UV light (λ=365 nm) for 3 minutes while under a nitrogen purge. The fully cured PFPE-DMA mold was then released from the silicon master. Separately, human serum albumin and trehalose was added in (25/75), (50/50), or (75/25) wt/wt to deionized water to make a 35 weight % solution. This mixture was spotted directly onto the contact point of the patterned PFPE-DMA mold and an unpatterned polyethyleneterethalate film affixed on a laminator. The stage of the laminator moved at a speed of 2.0 with 50 psi pressure put onto the roller. The solution filled mold was then placed in the freezer for at least four hours and subsequently lyophilized overnight. To the lyophilized filled mold was added 1 mL chloroform and with gentle mechanical force using a glass slide, the particles were extracted from the mold and harvested. Chloroform was slowly evaporated at ambient conditions to afford dry particles.

### Example 1.10

### Preparation of Transferrin Particles from Water

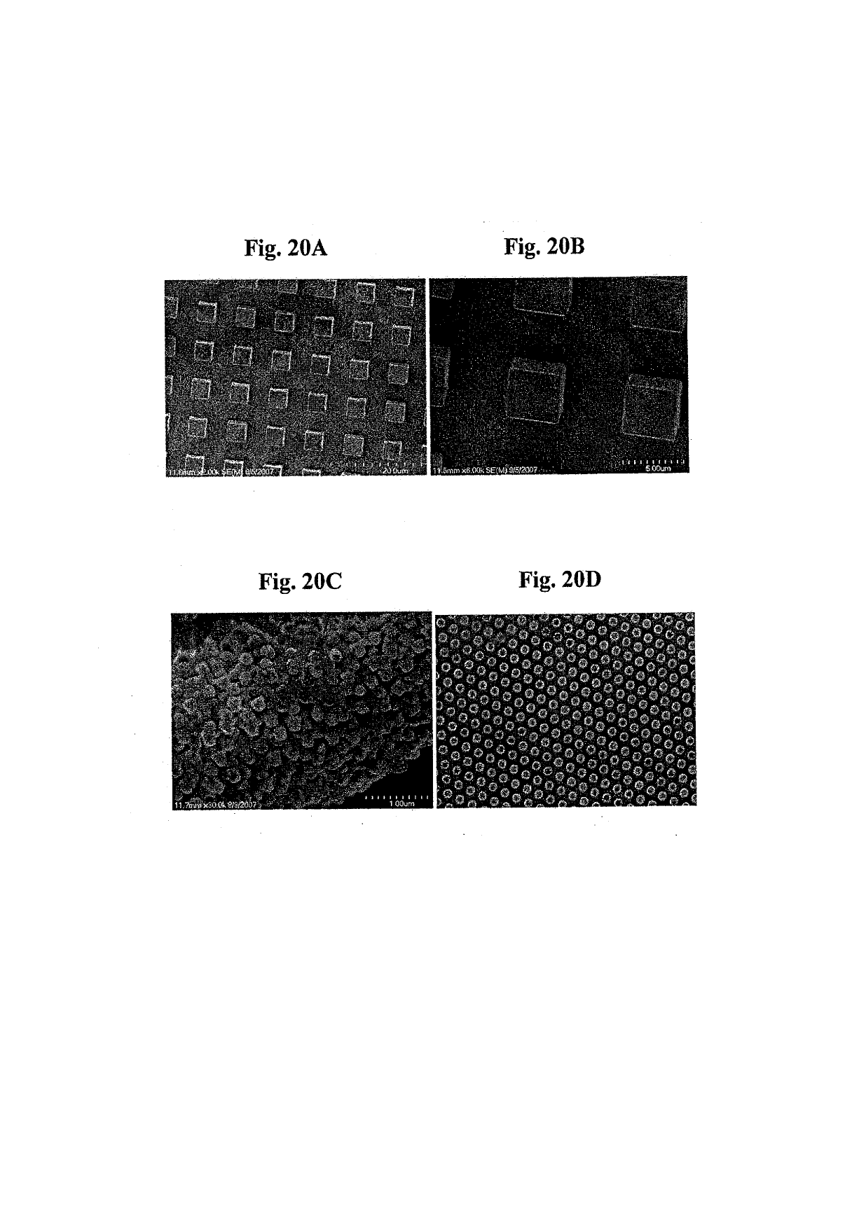

A patterned perfluoropolyether (PFPE) mold was generated by pouring a PFPE-dimethacrylate (PFPE-DMA) containing 1-hydroxycyclohexyl phenyl ketone over a silicon substrate patterned with 200 nm tall×200 nm diameter cylinders. The PFPE-DMA covered master was then subjected to UV light (λ=365 nm) for 3 minutes while under a nitrogen purge. The fully cured PFPE-DMA mold was then released from the silicon master. Separately, human transferrin was added to deionized water to make a 35 weight % composition. This mixture was spotted directly onto the contact point of the patterned PFPE-DMA mold and an unpatterned polyethyleneterethalate film affixed on a laminator. The stage of the laminator moved at a speed of 2.0 with 50 psi pressure put onto the roller. The solution filled mold was then placed in the freezer for at least four hours and subsequently lyophilized overnight. To the lyophilized filled mold was added 1 mL chloroform and with gentle mechanical force using a glass slide, the particles were extracted from the mold and harvested. Chloroform was slowly evaporated at ambient conditions to afford dry particles. Results are shown in FIGS. 20A-20D.

### Example 1.11

### Preparation of Insulin Particles

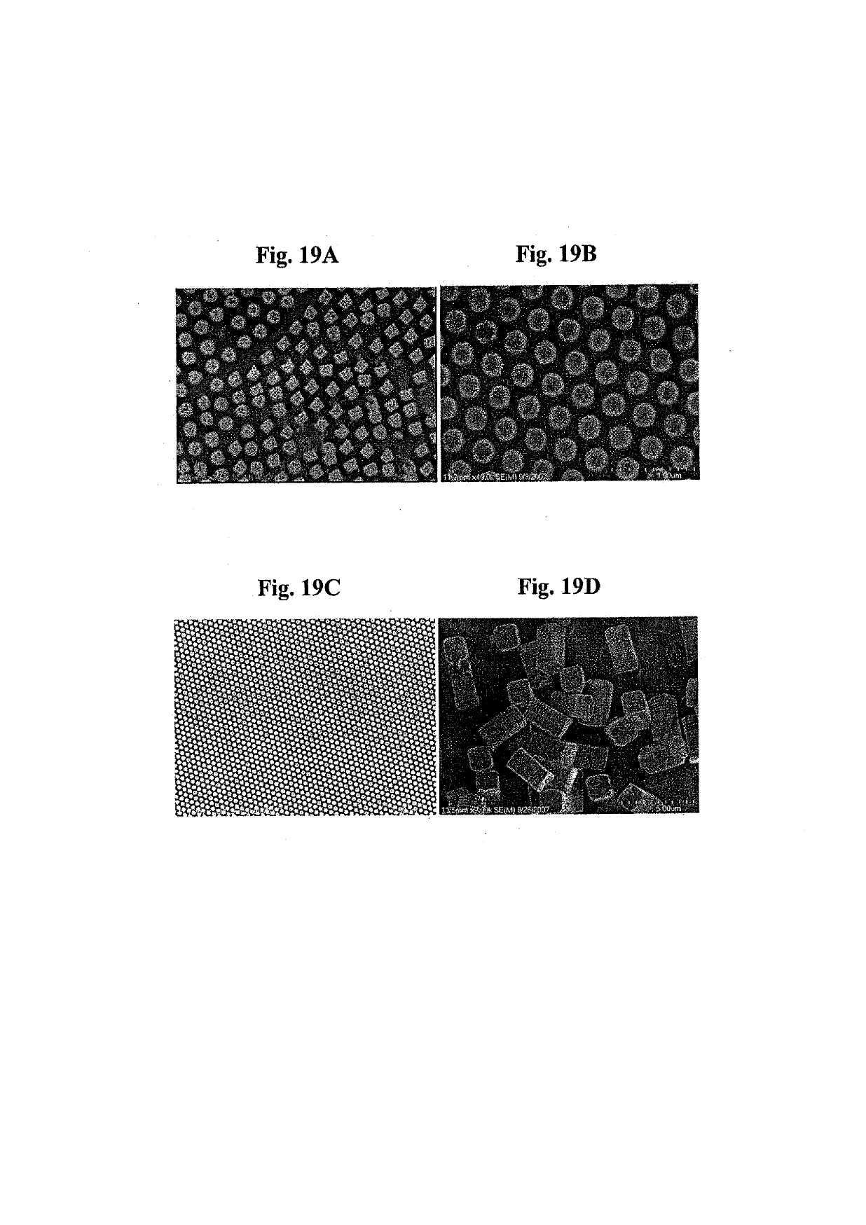

A patterned perfluoropolyether (PFPE) mold was generated by pouring a PFPE-dimethacrylate (PFPE-DMA) containing 1-hydroxycyclohexyl phenyl ketone over a silicon substrate patterned with 200 nm tall×200 nm diameter cylinders. A silicon substrate patterned with 4 μm tall×2 μm wide×2 μm wide cubes was also used. The PFPE-DMA covered master was then subjected to UV light (λ=365 nm) for 3 minutes while under a nitrogen purge. The fully cured PFPE-DMA mold was then released from the silicon master. Separately, human insulin was added to deionized water to make a 4 weight % composition. This mixture was vortexed briefly right before being spotted directly onto the contact point of the patterned PFPE-DMA mold and an unpatterned polyethyleneterethalate film affixed on a laminator. The stage of the laminator moved at a speed of 2.0 with 50 psi pressure put onto the roller. The mixture was spotted 2 more times onto the contact point of the patterned PFPE-DMA mold and an unpatterned polyethyleneterethalte film affixed to the laminator and passed through the pressurized roller. A fourth pass included solely the unpatterened polyethyleneterethalte film and the filled PFPE-DMA mold. The solution filled mold was then placed in the freezer for at least four hours and subsequently lyophilized overnight. Results are shown in FIGS. 19A-19D.

### Example 1.12

### Preparation of Interferon-Beta Particles

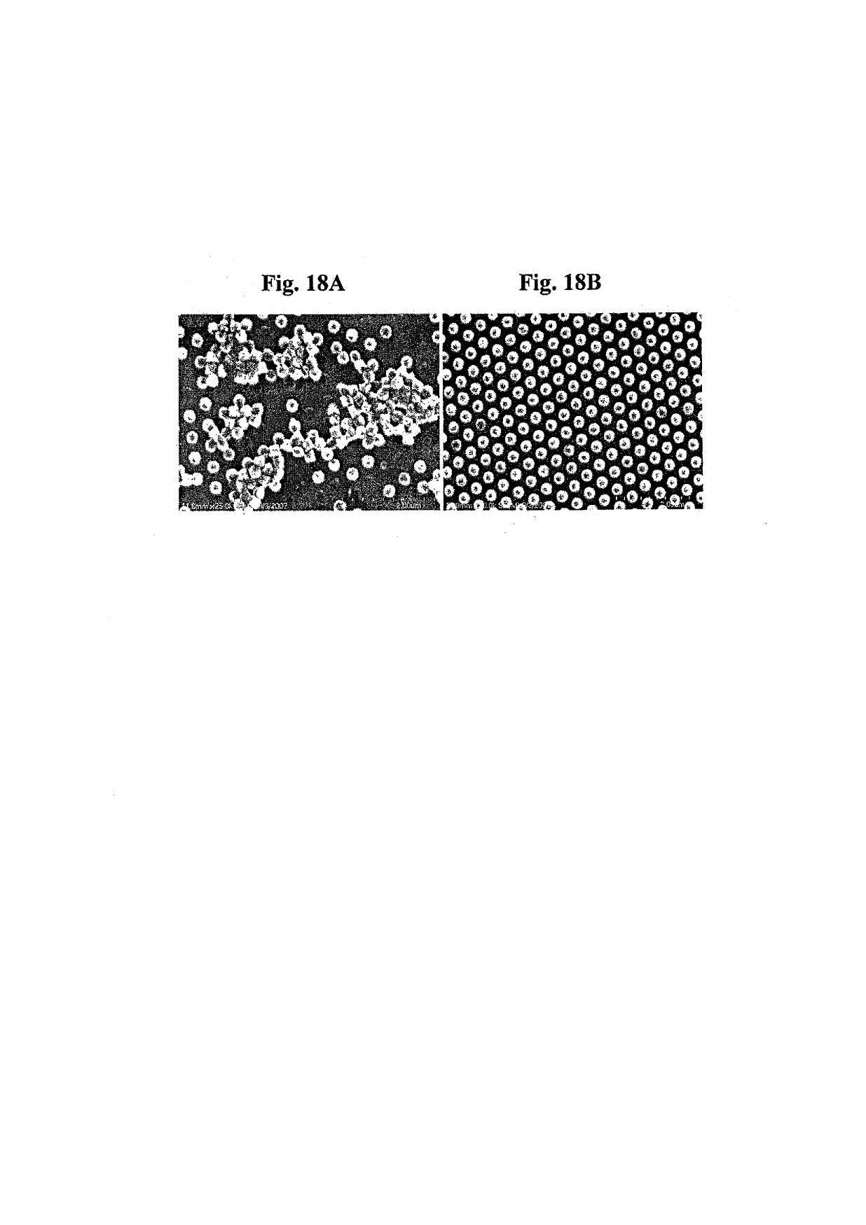

A patterned perfluoropolyether (PFPE) mold was generated by pouring a PFPE-dimethacrylate (PFPE-DMA) containing 1-hydroxycyclohexyl phenyl ketone over a silicon substrate patterned with 200 nm tall×200 nm diameter cylinders. The PFPE-DMA covered master was then subjected to UV light (λ=365 nm) for 3 minutes while under a nitrogen purge. The fully cured PFPE-DMA mold was then released from the silicon master. Separately, rat interferon-beta was added to deionized water to make a 10 weight % composition. This mixture was spotted directly onto the contact point of the patterned PFPE-DMA mold and an unpatterned polyethyleneterethalate film affixed on a laminator. The stage of the laminator moved at a speed of 2.0 with 50 psi pressure put onto the roller. The solution filled mold was then placed in the freezer for at least four hours and subsequently lyophilized overnight. Results are shown in FIGS. 18A-18B.

### Example 1.13

### Preparation of Hemoglobin Particles

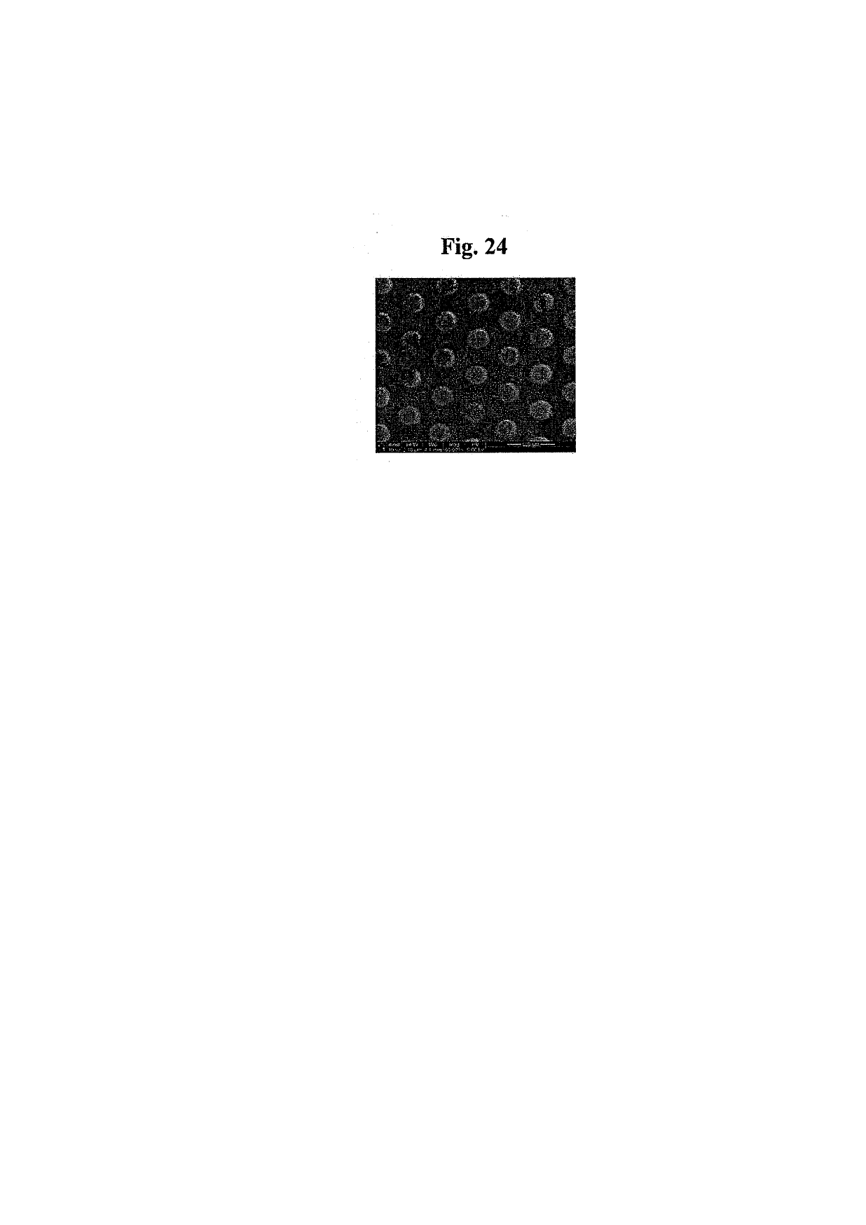

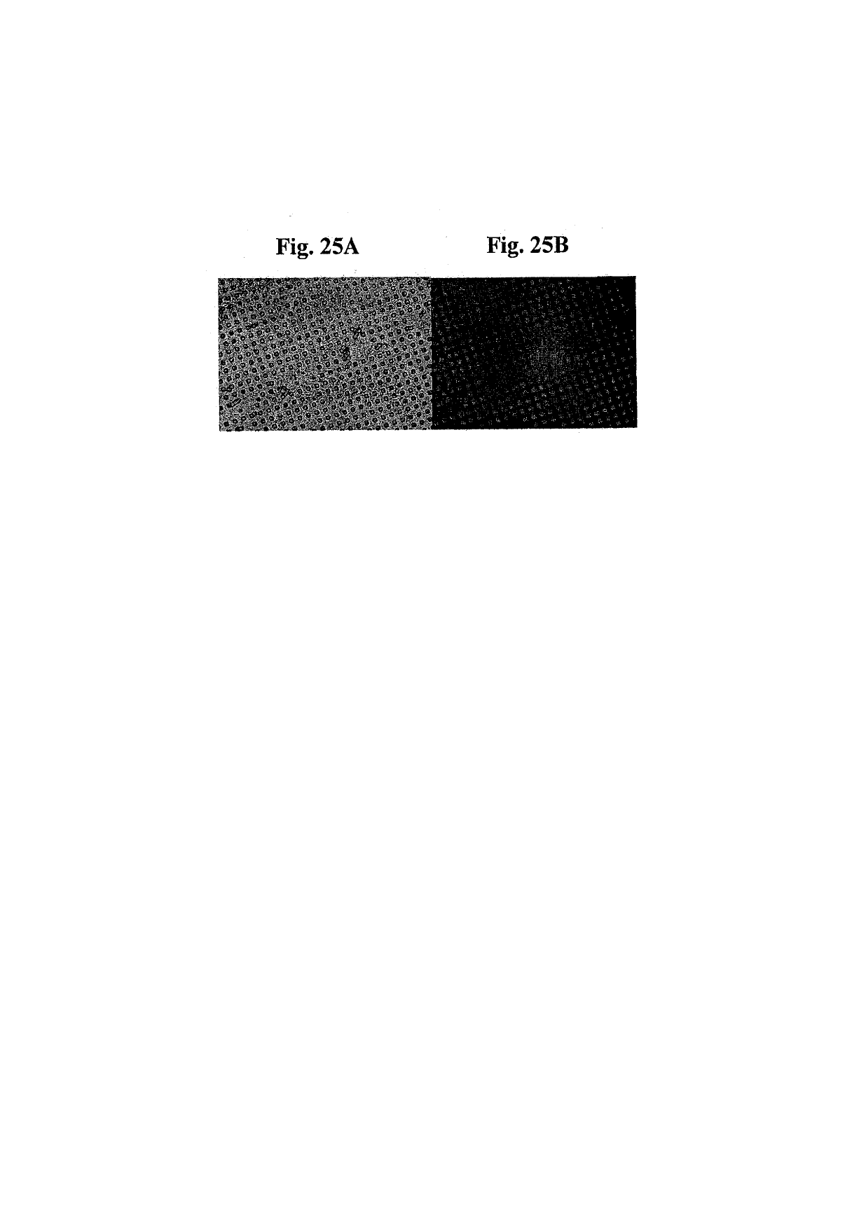

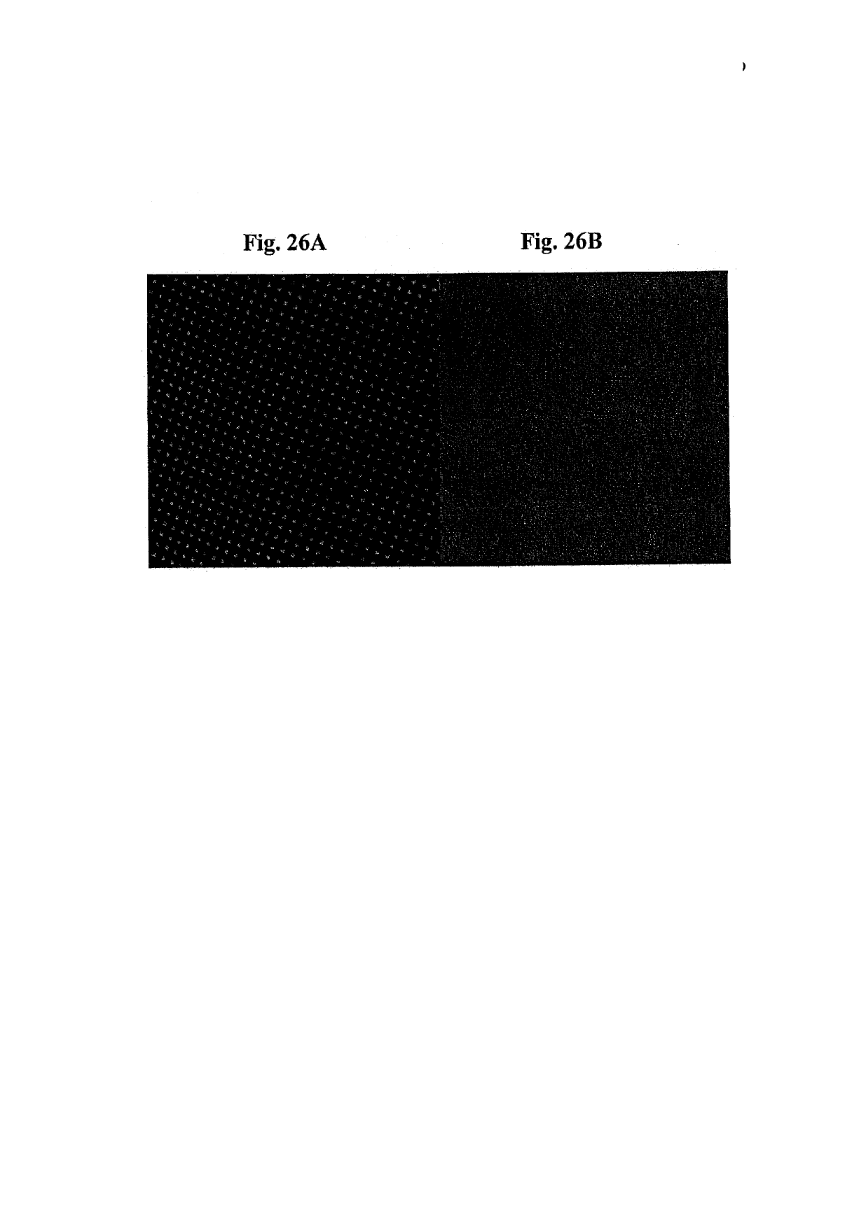

A patterned perfluoropolyether (PFPE) mold was generated by pouring a PFPE-dimethacrylate (PFPE-DMA) containing 1-hydroxycyclohexyl phenyl ketone over a silicon substrate patterned with 200 nm tall×200 nm diameter cylinders. A silicon substrate patterned with 5 μm tall×5 μm wide×5 μm wide cubes was also used. The PFPE-DMA covered master was then subjected to UV light (λ=365 nm) for 3 minutes while under a nitrogen purge. The fully cured PFPE-DMA mold was then released from the silicon master. Separately, human hemoglobin was added to deionized water to make a 25 weight % composition. This mixture was spotted directly onto the contact point of the patterned PFPE-DMA mold and an unpatterned polyethyleneterethalate film affixed on a laminator. The stage of the laminator moved at a speed of 2.0 with 50 psi pressure put onto the roller. The solution filled mold was then placed in the freezer for at least four hours and subsequently lyophilized overnight. Results are shown in FIGS. 24-26.

### Example 1.14

### Preparation of Albumin Particles

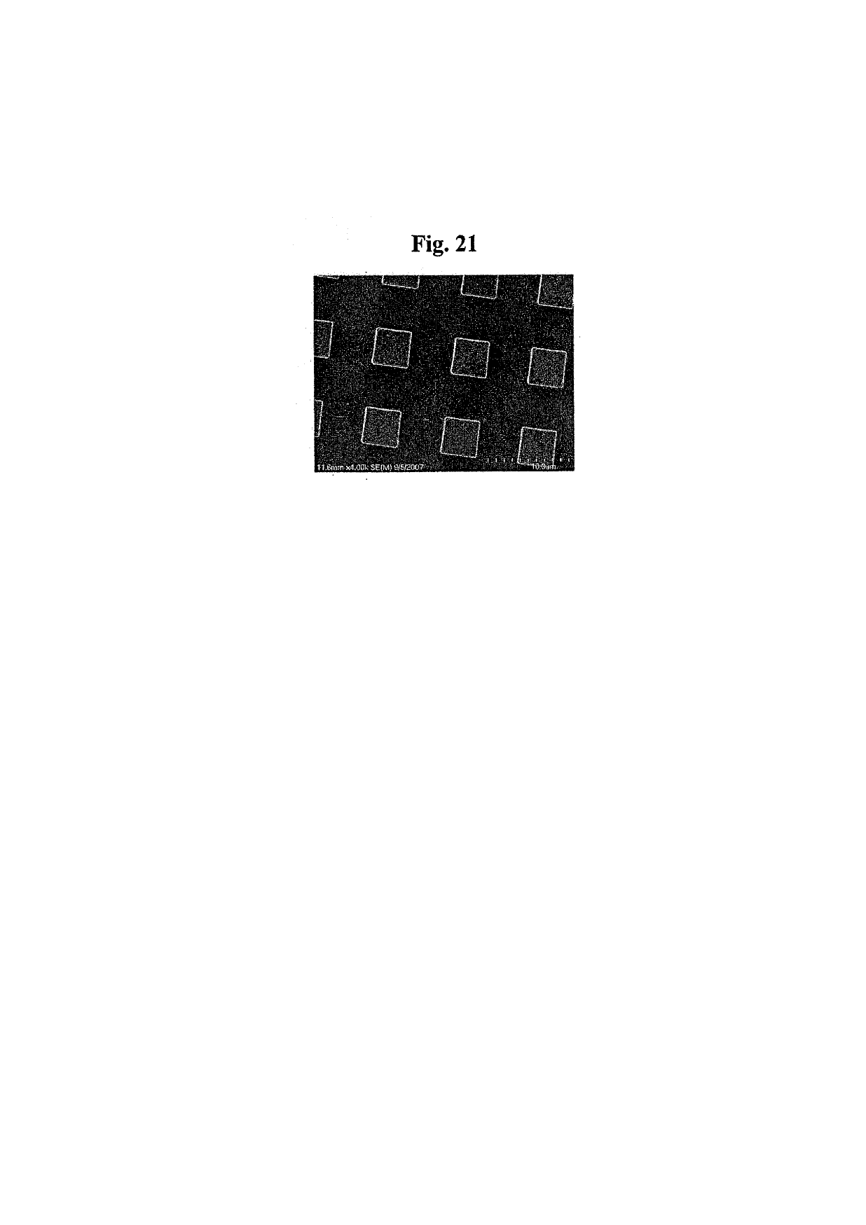

A patterned perfluoropolyether (PFPE) mold was generated by pouring a PFPE-dimethacrylate (PFPE-DMA) containing 1-hydroxycyclohexyl phenyl ketone over a silicon substrate patterned with 200 nm tall×200 nm diameter cylinders. A silicon substrate patterned with 5 μm tall×5 μm wide×5 μm wide cubes was also used. The PFPE-DMA covered master was then subjected to UV light (λ=365 nm) for 3 minutes while under a nitrogen purge. The fully cured PFPE-DMA mold was then released from the silicon master. Separately, human serum albumin was added to deionized water to make a 25 weight % composition. This mixture was spotted directly onto the contact point of the patterned PFPE-DMA mold and an unpatterned polyethyleneterethalate film affixed on a laminator. The stage of the laminator moved at a speed of 2.0 with 50 psi pressure put onto the roller. The solution filled mold was then placed in the freezer for at least four hours and subsequently lyophilized overnight. See, FIG. 21.

### Example 1.15

### Preparation of Transferrin Particles

A patterned perfluoropolyether (PFPE) mold was generated by pouring a PFPE-dimethacrylate (PFPE-DMA) containing 1-hydroxycyclohexyl phenyl ketone over a silicon substrate patterned with 200 nm tall×200 nm diameter cylinders. A silicon substrate patterned with 5 μm tall×5 μm wide×5 μm wide cubes was also used. The PFPE-DMA covered master was then subjected to UV light (λ=365 nm) for 3 minutes while under a nitrogen purge. The fully cured PFPE-DMA mold was then released from the silicon master. Separately, human transferrin was added to deionized water to make a 25 weight % composition. This mixture was spotted directly onto the contact point of the patterned PFPE-DMA mold and an unpatterned polyethyleneterethalate film affixed on a laminator. The stage of the laminator moved at a speed of 2.0 with 50 psi pressure put onto the roller. The solution filled mold was then placed in the freezer for at least four hours and subsequently lyophilized overnight. To the lyophilized filled mold was added 1 mL chloroform and with gentle mechanical force using a glass slide, the particles were extracted from the mold and harvested. Chloroform was slowly evaporated at ambient conditions to afford dry particles. Results are shown in FIGS. 20A-20D.

### Example 1.16

### Preparation of Gadolinium-Loaded Albumin Particles

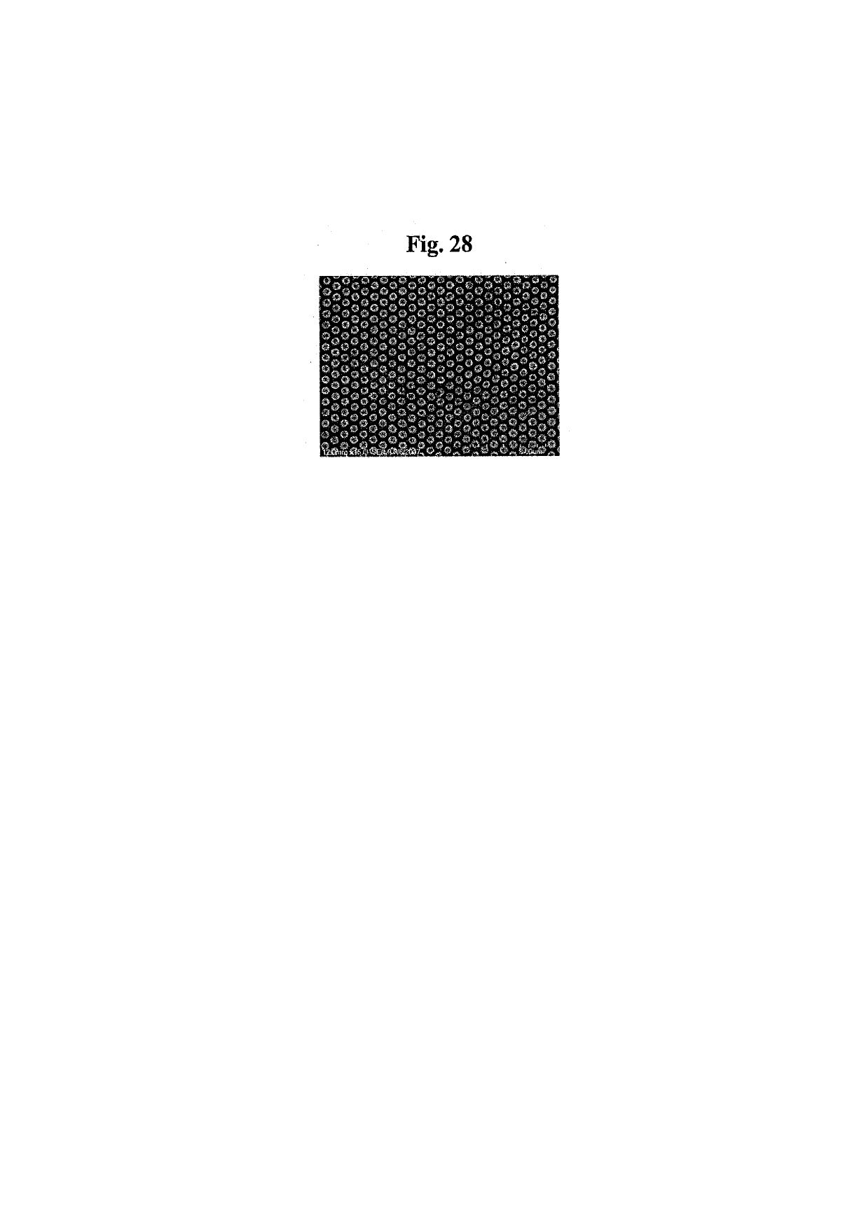

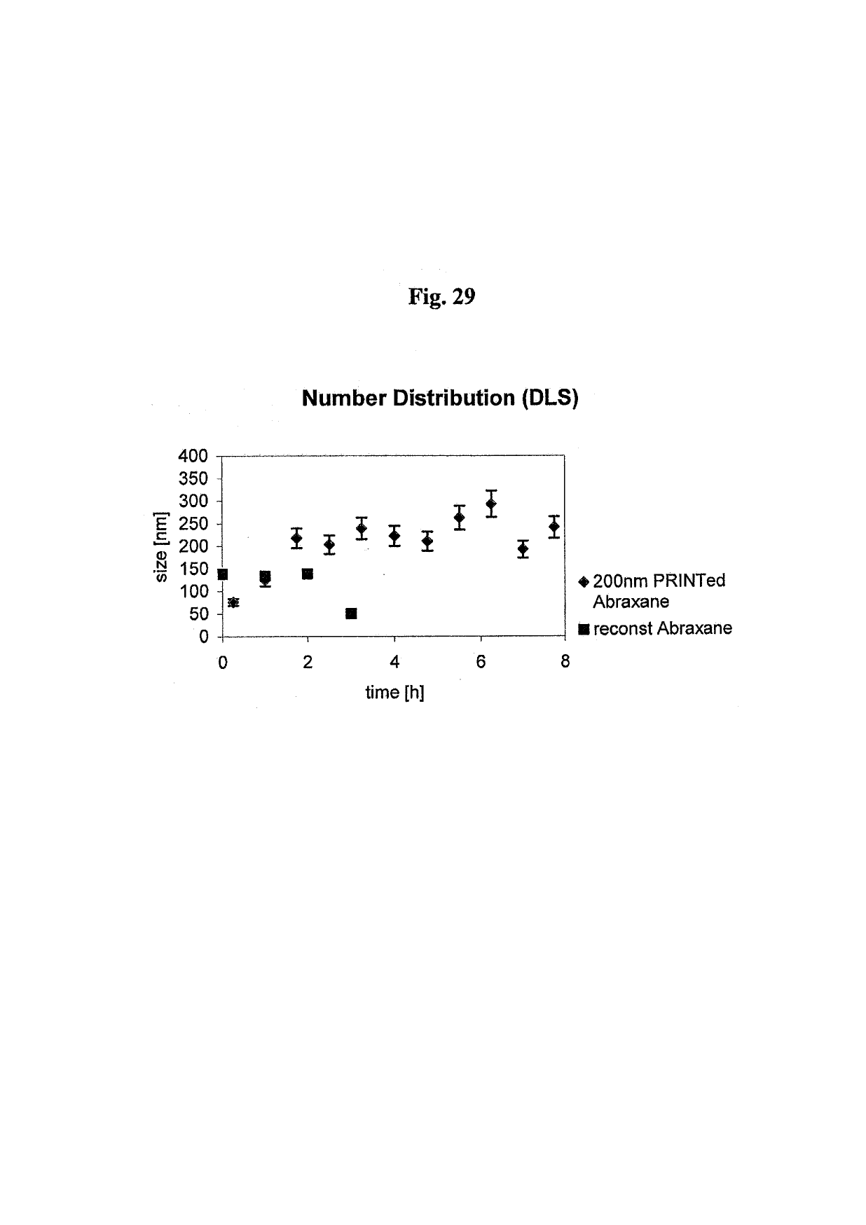

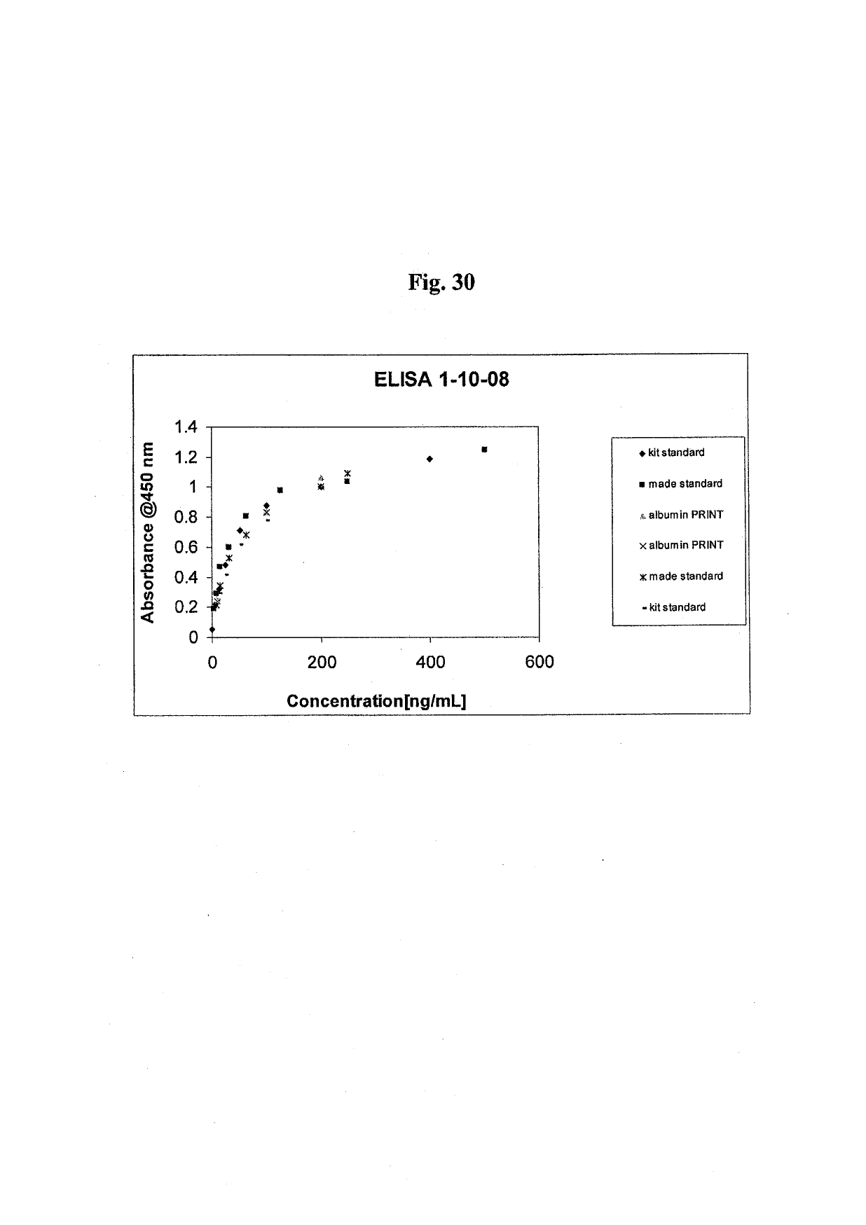

A patterned perfluoropolyether (PFPE) mold was generated by pouring a PFPE-dimethacrylate (PFPE-DMA) containing 1-hydroxycyclohexyl phenyl ketone over a silicon substrate patterned with 200 nm tall×200 nm diameter cylinders. The PFPE-DMA covered master was then subjected to UV light (λ=365 nm) for 3 minutes while under a nitrogen purge. The fully cured PFPE-DMA mold was then released from the silicon master. Separately, ethylene glycol coated gadolinium oxide was added to human serum albumin in equal parts. The mixture was added to deionized water to make a 35 weight % composition. This mixture was spotted directly onto the contact point of the patterned PFPE-DMA mold and an unpatterned polyethyleneterethalate film affixed on a laminator. The stage of the laminator moved at a speed of 2.0 with 50 psi pressure put onto the roller. The solution filled mold was then placed in the freezer for at least four hours and subsequently lyophilized overnight. To the lyophilized filled mold was added 1 mL chloroform and with gentle mechanical force using a glass slide, the particles were extracted from the mold and harvested. Chloroform was slowly evaporated at ambient conditions to afford dry particles. Results are shown in FIG. 28.

### Example 1.17

### Preparation of Horse Radish Peroxidase Particles

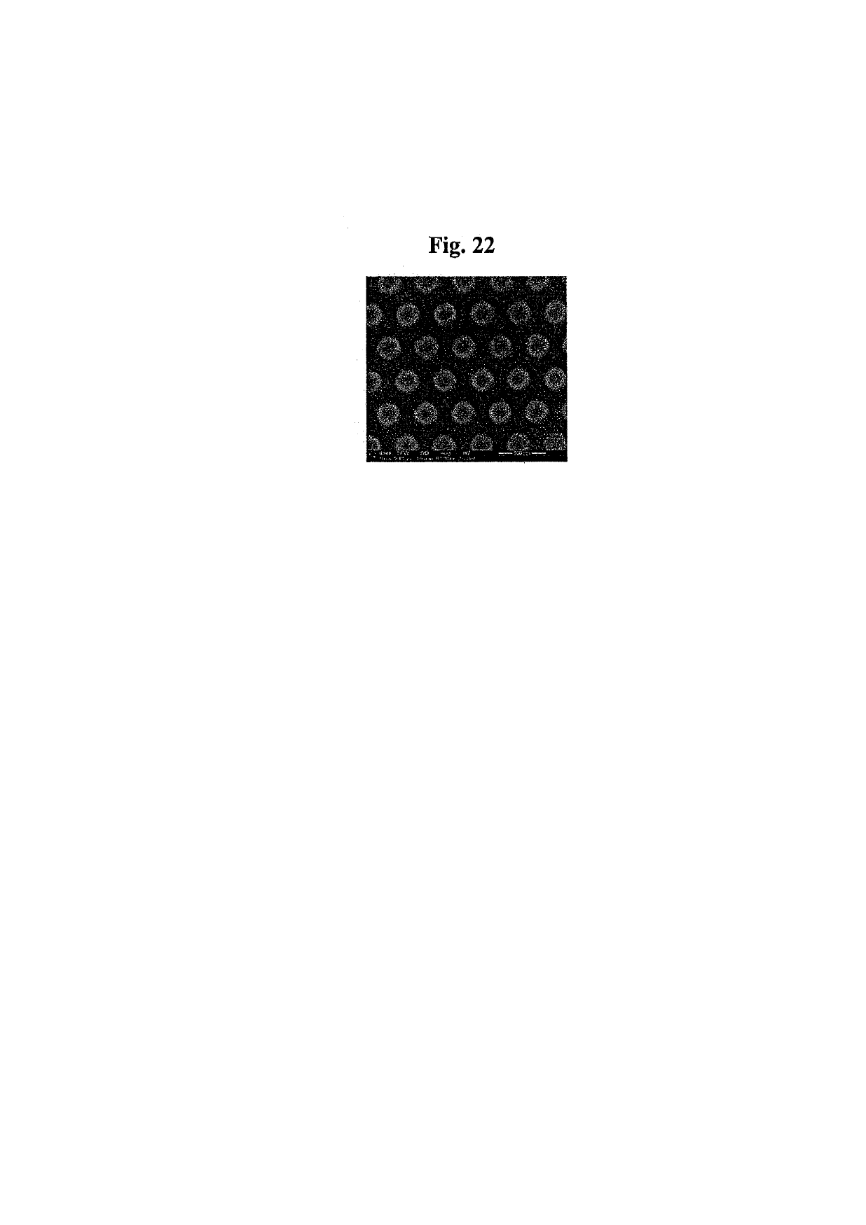

A patterned perfluoropolyether (PFPE) mold was generated by pouring a PFPE-dimethacrylate (PFPE-DMA) containing 1-hydroxycyclohexyl phenyl ketone over a silicon substrate patterned with 200 nm tall×200 nm diameter cylinders. The PFPE-DMA covered master was then subjected to UV light (λ=365 nm) for 3 minutes while under a nitrogen purge. The fully cured PFPE-DMA mold was then released from the silicon master. Separately, horse radish peroxidase was added to deionized water to make a 25 weight % composition. This mixture was spotted directly onto the contact point of the patterned PFPE-DMA mold and an unpatterned polyethyleneterethalate film affixed on a laminator. The stage of the laminator moved at a speed of 2.0 with 50 psi pressure put onto the roller. The solution filled mold was then placed in the freezer for at least four hours and subsequently lyophilized overnight. Results are shown in FIG. 22.

### Example 1.18

### Preparation of Trypsin Particles

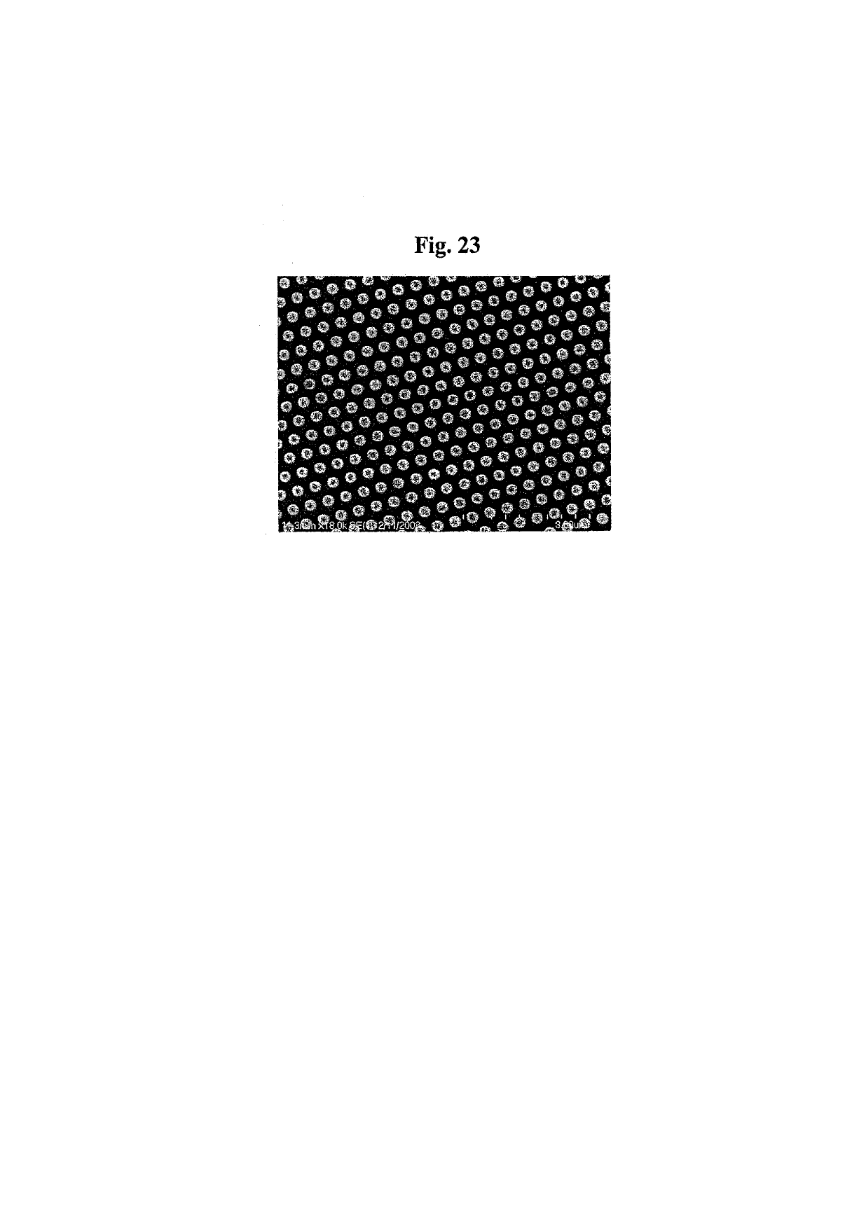

A patterned perfluoropolyether (PFPE) mold was generated by pouring a PFPE-dimethacrylate (PFPE-DMA) containing 1-hydroxycyclohexyl phenyl ketone over a silicon substrate patterned with 200 nm tall×200 nm diameter cylinders. The PFPE-DMA covered master was then subjected to UV light (λ=365 nm) for 3 minutes while under a nitrogen purge. The fully cured PFPE-DMA mold was then released from the silicon master. Separately, trypsin was added to deionized water to make a 25 weight % composition. This mixture was spotted directly onto the contact point of the patterned PFPE-DMA mold and an unpatterned polyethyleneterethalate film affixed on a laminator. The stage of the laminator moved at a speed of 2.0 with 50 psi pressure put onto the roller. The solution filled mold was then placed in the freezer for at least four hours and subsequently lyophilized overnight. Results are shown in FIG. 23.

### Example 1.19

### Preparation of Albumin with Fluorescent Dye Added Particles to Monitor Dissolution in Water

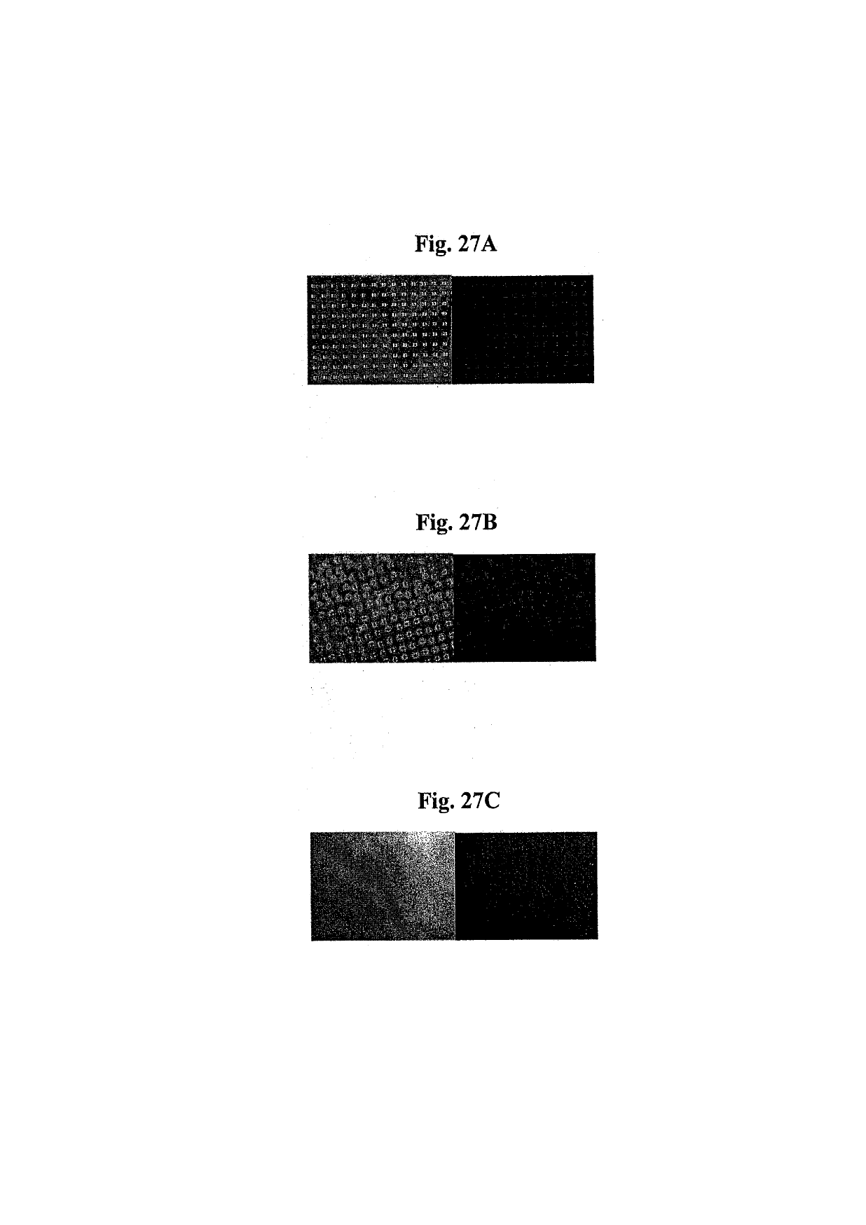

A patterned perfluoropolyether (PFPE) mold was generated by pouring a PFPE-dimethacrylate (PFPE-DMA) containing 1-hydroxycyclohexyl phenyl ketone over a silicon substrate patterned with 200 nm tall×200 nm diameter cylinders. The PFPE-DMA covered master was then subjected to UV light (λ=365 nm) for 3 minutes while under a nitrogen purge. The fully cured PFPE-DMA mold was then released from the silicon master. Separately, human serum albumin was added to a fluorescent dye, RhodamineB, in a 97.5/2.5 wt/wt ratio to deionized water to make a 25 weight % composition. This mixture was spotted directly onto the contact point of the patterned PFPE-DMA mold and an unpatterned polyethyleneterethalate film affixed on a laminator. The stage of the laminator moved at a speed of 2.0 with 50 psi pressure put onto the roller. The solution filled mold was then placed in the freezer for at least four hours and subsequently lyophilized overnight. A drop of 10 wt % poly(vinylpyrrolidone) (PVP) in water was added to a substrate (glass or untreated PET) and spread onto the substraight using a Meyer Rod. The filled, lyophilized mold was placed atop the PVP solution, pattern down, and rolled out onto the spread PVP solution. Once the PVP had dried, the mold was lifted, yielding particles harvested onto the excipient (PVP) layer. Dissolution of protein particles are monitored using optical microscopy as water is added the harvested film. Results are shown in FIGS. 27A-27C.

### Example 1.20

### Preparation of Goat Anti-Human Immunoglobin G Antibody (IgG) Particles

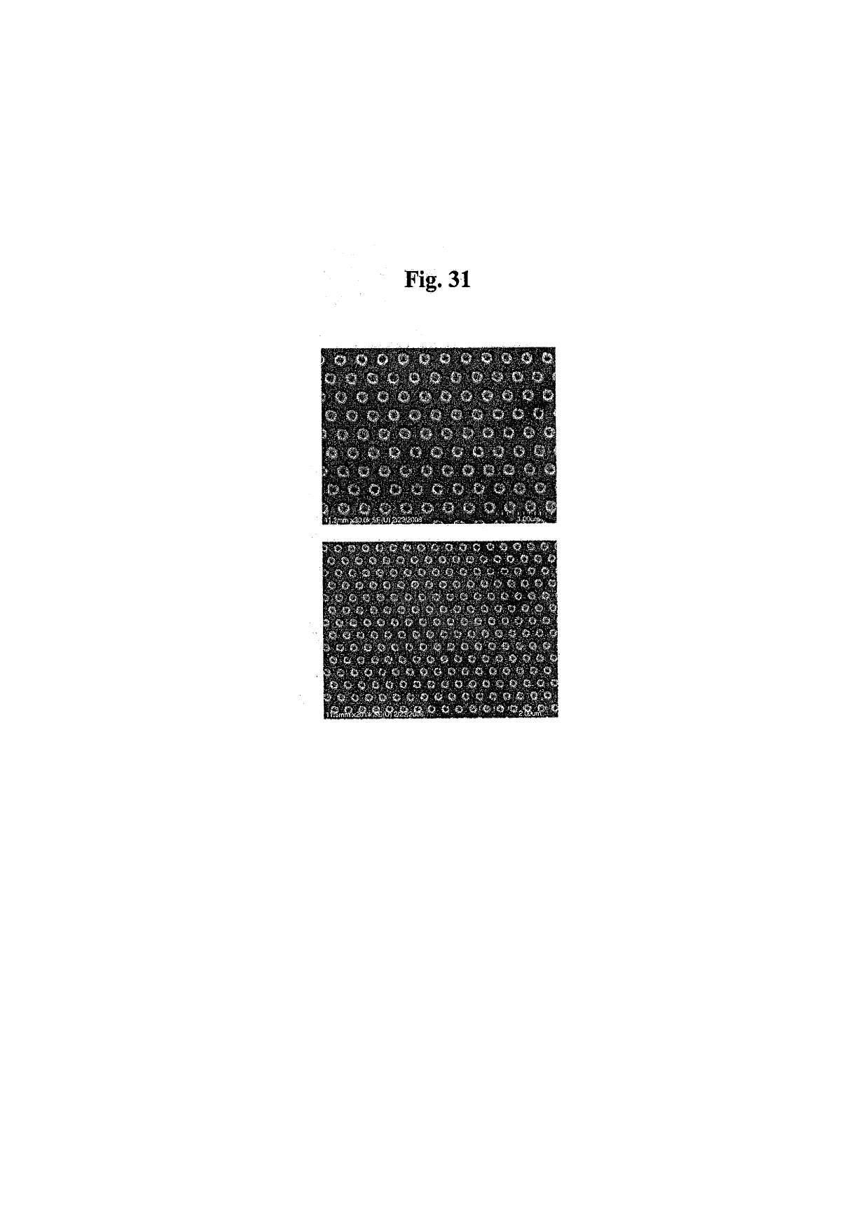

A patterned perfluoropolyether (PFPE) mold was generated by pouring a PFPE-dimethacrylate (PFPE-DMA) containing 1-hydroxycyclohexyl phenyl ketone over a silicon substrate patterned with 200 nm tall×200 nm diameter cylinders. The PFPE-DMA covered master was then subjected to UV light (λ=365 nm) for 3 minutes while under a nitrogen purge. The fully cured PFPE-DMA mold was then released from the silicon master. Separately, affinity-purified goat anti-human immunoglobin G antibody (used as received from Pel-Freez) as a 7.90 mg/mL solution in 10 mM sodium phosphate, 0.15M sodium chloride, 0.05% (w/v) sodium azide, pH 7.2 and filtered through 0.2 um filter was spotted directly onto the contact point of the patterned PFPE-DMA mold and an unpatterned polyethyleneterethalate film affixed on a laminator. The stage of the laminator moved at a speed of 2.0 with 50 psi pressure put onto the roller. Spotting the solution onto a new contact point was repeated three more times. The solution filled mold was then placed in the freezer for at least four hours and subsequently lyophilized overnight. Separately, a drop of cyanoacrylate was added to a substrate (glass or untreated PET) and a filled, lyophilized mold was placed atop the cyanoacrylate drop, pattern down, and rolled out such that the drop underneath the mold spread. Once the cyanoacrylate was polymerized, the mold was lifted, yielding particles harvested onto the adhesive layer. Individual particles are obtainable by dissolving the adhesive layer with acetone. See, FIG. 31.

### Example 2.1

### Preparation of Paclitaxel Encapsulated Albumin PRINT Particles

A patterned perfluoropolyether (PFPE) mold will be generated by pouring a PFPE-dimethacrylate (PFPE-DMA) containing 1-hydroxycyclohexyl phenyl ketone over a silicon substrate patterned with 200 nm tall×200 nm diameter cylinders. The PFPE-DMA covered master will then be subjected to UV light (λ=365 nm) for 3 minutes while under a nitrogen purge. The fully cured PFPE-DMA mold will then be released from the silicon master. Separately, 9 mg of paclitaxel will be added to 200 μL of 1:1 v/v water/ethyleneglycol, upon which 77 mg human serum albumin will be added. This mixture will then be spotted directly onto the contact point of the patterned PFPE-DMA mold and an unpatterned polyethyleneterethalate film affixed on a laminator. The stage of the laminator will move at a speed of 2.0 with 30 psi pressure put onto the roller. The solution filled mold will be then placed in a protective area on the bench top overnight for slow solvent evaporation.

### Example 2.2

### Preparation of Modified Albumin PRINT Particles after Molding and Harvesting Using Water Insoluble Crosslinkers

A patterned perfluoropolyether (PFPE) mold will be generated by pouring a PFPE-dimethacrylate (PFPE-DMA) containing 1-hydroxycyclohexyl phenyl ketone over a silicon substrate patterned with 200 nm tall×200 nm diameter cylinders. The PFPE-DMA covered master will then be subjected to UV light (λ=365 nm) for 3 minutes while under a nitrogen purge. The fully cured PFPE-DMA mold will then be released from the silicon master. Separately, a 35 wt % solution of albumin in 1:1 v/v water/ethyleneglycol will be made and applied to the contact point of the patterned PFPE-DMA mold and an unpatterned polyethyleneterethalate film affixed on a laminator. After slow evaporation overnight, water insoluble crosslinker such as dithiobis[succinimidylpropionate] (DSP) or disuccinimidyl suberate (DSS) will be added onto the mold. After sufficient reaction time on the surface, excess crosslinker will be washed away. Particle will be then harvested on a medical adhesive layer or an excipient layer. Water-insoluble crosslinker will be added to the harvest layer and allowed to react with the remainder of particle surfaces. Particles will then be washed off the harvest layer and collected and purified by filtration after the harvesting layer is dissolved.

### Example 2.3

### Preparation of Modified Albumin PRINT Particles after Molding and Harvesting Using Water Soluble Crosslinkers

A patterned perfluoropolyether (PFPE) mold will be generated by pouring a PFPE-dimethacrylate (PFPE-DMA) containing 1-hydroxycyclohexyl phenyl ketone over a silicon substrate patterned with 200 nm tall×200 nm diameter cylinders. The PFPE-DMA covered master will then be subjected to UV light (λ=365 nm) for 3 minutes while under a nitrogen purge. The fully cured PFPE-DMA mold will then be released from the silicon master. Separately, varying wt % ratios of a water soluble crosslinker such as 3,3′-Dithiobis(sulfosuccinimidylpropionate) (DTSSP) will be added to human albumin serum to make an overall 35 wt % solution on 1:1 v/v water/ethyleneglycol. The solution will then be quickly applied to the contact point of the patterned PFPE-DMA mold and an unpatterned polyethyleneterethalate film affixed on a laminator, and run to obtain a filled mold. After slow evaporation overnight, particles will either be harvested via the direct scraping method or using an adhesive or excipient layer.

### Example 2.4

### Investigation of Pharmacologically Active Agent (PAA) Loading in Albumin PRINT Particles

Compositions containing approximately 35 wt % solids in 1:1 v/v water/ethyleneglycol, will contain varying PAA loading from 0.5% to 100% with respect to albumin. The PAA will consists of hydrophilic as well as hydrophobic agents including, but not limited to, siRNA, paclitaxel, doxorubicin, Sirolimus, enzymes, and protease. The solution will then be applied to the contact point of the patterned PFPE-DMA mold and an unpatterned polyethyleneterethalate film affixed on a laminator, and run to produce a filled mold. After slow evaporation overnight, particles will either be harvested via the direct mechanical method or using an adhesive or excipient layer.

### Example 2.5

### Investigation of Crosslinking Agents and Crosslink Density in Albumin PRINT Particles

One of two compositions will contain 35 wt % solids in 1:1 v/v water/ethyleneglycol, but not limited to that % solids, will contain water-soluble crosslinkers of slow reaction kinetic of varying ratios to albumin including, but not limited to, 0.25-100% to albumin. The solution will then be applied to the contact point of the patterned PFPE-DMA mold and an unpatterned polyethyleneterethalate film affixed on a laminator, and run to obtain a filled PFPE mold. After slow evaporation overnight, particles will either be harvested via the direct mechanical method or using an adhesive or excipient layer. The other compositions will not contain a crosslinker in the formulation, but once albumin particles are in the mold, surface crosslinking will be achieved with a water-insoluble crosslinker. These particles will be harvested onto an adhesive or excipient layer followed by crosslinking the rest of the albumin particle surfaces. All crosslinkers investigated will contain, but not limited to, linkages that degrade in reducing environments, acid labile linkages, or stable linkages to the former two environments.

### Example 2.6

### Investigation of Targeting Albumin PRINT Particles

Post-functionalization of albumin PRINT particles for targeting specific cells, organs, tumors, and other human and animal tissue will include, but not limited to, avidin/biotin complex, Mabs, targeting peptides, and aptamers. Particle surface functionalization will include, but not limited to, reacting primary amines, alcohols, carboxylic acids, thiols, or other moieties contained in albumin with CDI or the like for further functionalization with any nucleophile or electrophile.

Since these are proteins, depending on how they are crosslinked, targeting moieties can be attached through free amines or thiols on the surface. It is likely that an amine-reactive targeting ligand or a thiol-reactive targeting ligand will be used.

### Example 2.7

### Investigation of Size and Shape of Albumin PRINT Particles

Albumin PRINT particles of varying composition and encapsulating a variety of pharmacologically active agents will be molded using PFPE-DMA molds made from patterned silicon substrates. The patterns will include but are not limited to, cylinders of diameter equal to 200 nm, 500 nm, and 1000 nm having aspect ratios of 0.5, 1.0, 2.0, and 3.0.

### Example 2.8

### Investigation of Lyophilization of Albumin PRINT Particles

Albumin PRINT particles will be lyophilized once compositions thereof have filled the mold, instead of slow solvent evaporation. After lyophilization, particles will be harvested using one of a number of harvesting methods afore mentioned. Separately, harvested particles will be lyophilized prior to reconstitution in for example water or saline. Albumin PRINT particles in the mold, on and adhesive or excipient layer, or modified and in aqueous solution can be obtained by lyophilization and serve as a cryoprotectant and reconstitution aid. Albumin PRINT particles will be harvested, with and without filtration through Fisher P8 20-25 μm filter pore size, followed by drying or lyophilization to produce a sterile solid formulation useful for intravenous injection.

### Example 2.9

### Incorporation of Pharmacologically Active Agent Post Protein Particle Formation with or without Modification

Polymer particles composed of, but not limited to, a protein such as albumin, unmodified or modified by, but not limited to, chemical crosslinking will be exposed to a concentrated solution containing, but not limited to, a pharmacologically active agent (PAA) such as taxol after direct harvesting or harvesting onto an adhesive or excipient layer. The exposure will have a duration of minutes to days depending on a rate of diffusion of the PAA into the polymer particles. The polymer particles with PAA diffused therein and encapsulated within will then be washed of excess solution containing the PAA. The amount of PAA encapsulated can be determined by the difference of the loaded polymer particle weight and the pre-loaded polymer particle weight, respectively. Subsequently, the loaded polymer particles will be dried or lyophilized prior to reconstitution into water or saline depending on particular applications.

### Example 3.1

### Molding of Transferrin at the 200 Nm×200 Nm Cylinder Scale

A patterned perfluoropolyether (PFPE) mold was generated by pouring a PFPE-dimethacrylate (PFPE-DMA) containing 2,2′-diethoxyacetophenone over a silicon substrate patterned with 200×200 nm cylinders. The PFPE-DMA covered master was then subjected to UV light (λ=365 nm) for 2 minutes while under a nitrogen purge. The fully cured PFPE-DMA mold was then released from the silicon master. Separately, a 33 wt % solution of human transferrin was prepared by dissolving 10.3 mg of human transferrin (Aldrich) into 20 μL of phosphate buffered saline (pH 7.2). This solution (10 μL) was spotted across one end of the mold, and then spread across the mold by rolling a PET film across the surface under pressure. The PET film was slowly removed from the mold with transfer the patterned protein film to the PET. Analysis by SEM shows 200×200 nm cylindrical transferrin posts on a ˜500 nm thick layer of transferrin. Free particles are also observed, as shown in FIGS. 15 and 16.

### Example 4.1

### Polydispersity Calculations, Examples, and Comparative Examples

Dynamic light scattering (DLS) measures the intensity fluctuations with time and correlates these fluctuations to the properties of the scattering objects, presented as autocorrelation function g⁽²⁾(q,t) of the scattering intensity. The autocorrelation function depends on how molecules move on the length scale 1/q, with a characteristic time τ.

$$τ = \frac{1}{{Dq}^{2}}$$

where D is the transitional diffusion coefficient, The scattering wave vector q is given by

$$q = \frac{4 π {n}_{s}}{λ} sin ( Θ / 2 )$$

where nₛ is the refractive index of the solvent, λ is the wavelength of the light in the vacuum and Θ is the scattering angle.

The particle sizes are calculated from transitional diffusion coefficient by Stroke-Einstein Equation.

$$D = \frac{{k}_{B} T}{3 π η ( t ) d}$$

where ηₛ is the solvent viscosity, k_{B} is the Boltzmann constant, T is the absolute temperature and Rₕ is the hydrodynamic radius. For samples with broad unimodal or multimodal size distribution, DLS data was analyzed by the Non-Negative Constrained Lease Squares (NNLS) (I. Morrison, E. Grabowski, and C. Herb, Langmuir, 1 (1985) 496) and integral transform method CONTIN (S. Provencher, Computer Phys. Comm. 27 (1982) 213 and 229) to obtain size and size distribution.

The polyidspersity of particles were calculated by Cumulant Analysis (D. Koppel, J. Chem. Phys., 57 (1972) 4814). The statistic deviation of diffusion coefficient is (based on the band width of lognormal plot)

$$Polydispersity = {μ}_{2} / {τ}^{2} = \frac{( {D}^{2} - {\overline{D}}^{2} )}{{D}^{2}}$$

μ₂ is proportional to the variations of the “intensity” weighed diffusion coefficient distribution and carries the information of the width of the size distribution. D̅ is the average diffusion coefficient. Polydispersity has no unit and has been reported as the indication of size distribution of colloids, particles. (Common concept in medical, biological and colloid literature)

| Polydispersity | Interpretation |
|---|---|
| 0-0.02 | monodispere |
| 0.02-0.08 | narrow disperse |
| >0.08 | broad disperse |

### Example 4.2

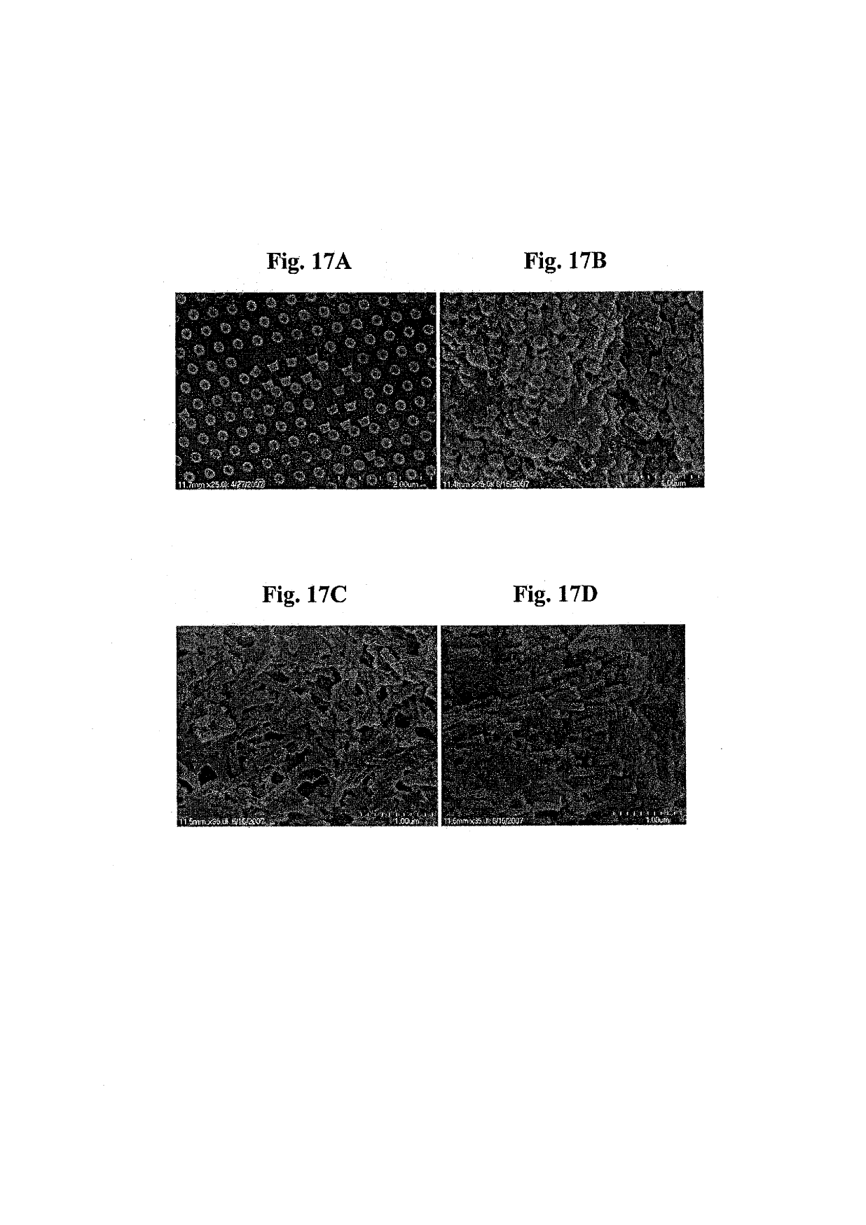

Abraxane™ nanoparticles of varying sizes and aspect ratios are fabricated. A patterned perfluoropolyether (PFPE) mold was generated by pouring a PFPE-dimethacrylate (PFPE-DMA) containing 1-hydroxycyclohexyl phenyl ketone over a silicon substrate patterned with 200 nm tall×200 nm diameter cylinders. A silicon substrate patterned with 600 nm tall×200 nm diameter cylinders was also used. The PFPE-DMA covered master was then subjected to UV light (λ=365 nm) for 3 minutes while under a nitrogen purge. The fully cured PFPE-DMA mold was then released from the silicon master. Separately, Abraxane was added to deionized water to make a 25 weight % composition. This mixture was spotted directly onto the contact point of the patterned PFPE-DMA mold and an unpatterned polyethyleneterethalate film affixed on a laminator. The stage of the laminator moved at a speed of 2.0 with 50 psi pressure put onto the roller. The solution filled mold was then placed in the freezer for at least four hours and subsequently lyophilized overnight. To the lyophilized filled mold was added 1 mL harvesting solvent and with gentle mechanical force using a glass slide, the particles were extracted from the mold and harvested. Dynamic light scattering for 200 nm PRINTed Abraxane showed the size of the PRINT Abraxane was approximately 230 nm and the size of the particles was stable for 6 hours at 37° C. in saline compared to the reconstituted Abraxane which has a solution stability of approximately three hours. Significant improvements in the solution stability should translate into improved lifetime in vivo and increased efficacy. See, FIG. 17A-17D.

### Example 4.3

### Treatment of Metastatic Breast Carcinoma

Albumin/paclitaxel particles are prepared as described in Example 4.2 and formulated in a saline solution to form a pharmaceutical composition. The pharmaceutical composition is administered as a 30-minute infusion at a dose in the range of 175 mg/m² to 300 mg/m² to patients with metastatic breast cancer. The pharmaceutical compositions are administered at 3 week intervals. Control patients received paclitaxel injection at 175 mg/m² given as a 3-hour infusion.

Patients receiving the albumin/paclitaxel particles are expected to have a statistically significantly higher reconciled target lesion response rate than patients receiving paclitaxel injection.

### Example 5.1

### ELISA Assay of Free Protein and Protein PRINT Particles for Biological Function Determination

A standard ELISA assay was conducted on free albumin and albumin PRINT particles. (Product reference and protocol reference: E101; http://www.bethyl.com/) Protein solutions were in the suggested working range (5-500 ng/mL) for both free protein and protein PRINT particles. Protein PRINT particles were harvested directly using buffer. In the case of human albumin free protein and protein PRINT particle, an anti-human albumin polyclonal antibody was used and the respective capture antibody in the ELISA. The results from the ELISA assay strongly suggest that the nanomolded protein retains biological structure and function following the molding process.

### Example 5.2

### Enzymatic Activity Assay to Measure Activity of Free Horseradish Peroxidase and PRINT Particles Thereof

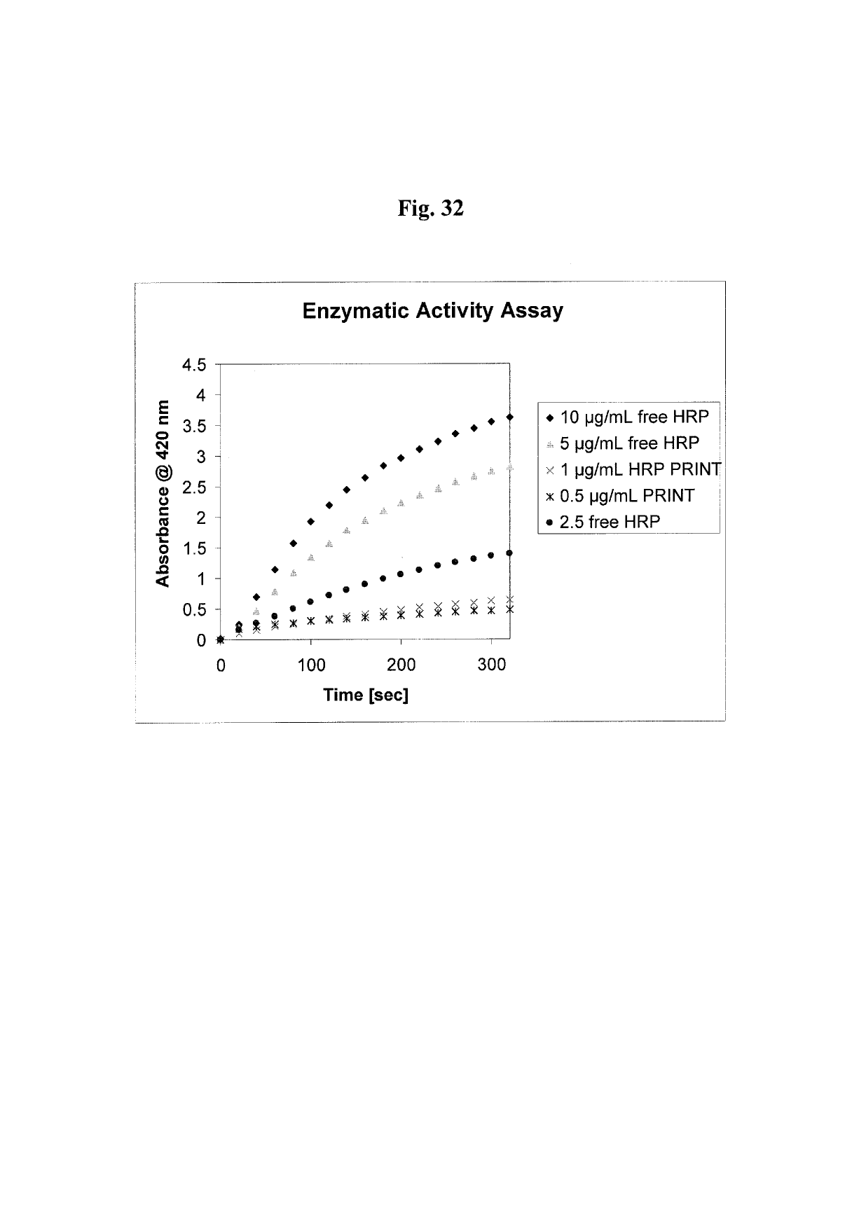

A standard enzymatic assay of horseradish peroxidase (reference P6782; Sigma Aldrich) was conduted on free horseradish peroxidase and PRINT particles thereof in the concentration range of 10 ng/mL-10 mg/mL in buffer. The substrates used were hydrogen peroxide and pyrogallol. The results from the ELISA assay strongly suggest that the nanomolded protein retains biological structure and function following the molding process. See, FIG. 32.

### 2. Experiments

Dynamic scattering studies were performed using a 90Plus Particle Analyzer (Brookhaven Instruments) with 30 mW laser source. Data collection was performed at a detection angle of 90° and typical sample volume of 2 mL. The experiments temperatures were controlled by the heating system integrated inside the 90Plus. The scattering data of the samples was analyzed by Cumulant analysis to obtain polydispersity, by COTIN and NNLS methods to obtain size and distribution. Samples of DLS experiments were dissolved in distilled water, buffers or 0.9 wt % NaCl aqueous solution at concentrations from 0.01 to 1 mg/mL. To minimize dust interference, all solutions were freshly prepared.

### Abraxane® Comparative Example 1

5 mg of Abraxane® (Abraxis BioScience, Inc. and AstraZeneca) was dissolved in 100 mL 0.9 wt % NaCl aqueous solution to get the concentration at 0.5 mg/mL. 2 mL of Abraxane® solution was put inside a cubic light scattering cell and the DLS experiments was performed at 25° C. The solvent refractive index was set as water and dust cut off ratio was 80%. The experiment duration time was 1 minute and each sample was measured 5 times.

### Abraxane® Comparative Example 2

5 mg of Abraxane® was dissolved in 100 mL 0.9 wt % NaCl aqueous solution to get the concentration at 0.5 mg/mL. 2 mL of Abraxane® solution was put inside a cubic light scattering cell and then the solution was heated up to 37° C. The DLS experiment was performed at 37° C. The solvent refractive index was set as water and dust cut off ratio was 80%. The experiment duration time was 1 minute and each samples was measured 5 times.

1. A pharmaceutical composition comprising;
a plurality of monodisperse micro and/or nanoparticles said particles having predetermined geometric shapes and a broadest dimension less than about 10 micrometers,
the particles comprising protein; and
wherein the particles substantially retain the predetermined geometric shape for at least four hours at about 37° C. in saline.

2. The pharmaceutical composition of claim 1, wherein said particles further comprise an active agent.

3. The pharmaceutical composition of claim 1, wherein the particles substantially retain the predetermined geometric shape for more than five hours at about 37° C. in saline.

4. The pharmaceutical composition of claim 2, wherein said active agent is an active hydrophobic pharmaceutical agent or an active hydrophilic pharmaceutical agent.

5. The pharmaceutical composition of claim 1, wherein said protein is selected from the group consisting of a therapeutic protein, a diagnostic protein, or a monoclonal antibody.

6. The pharmaceutical composition of claim 2, wherein said active agent is a taxane, paclitaxel, siRNA, doxorubicin, rapamyacin, sirolimus, an antisense oligonucleotide, an enzyme, protease, a chemotherapeutic, an antiinfective agent, or an immunosuppressive agent.

7. The pharmaceutical composition of claim 1, wherein the protein is albumin.

8. The pharmaceutical composition of claim 7, wherein said protein is albumin and said active agent is paclitaxel.

9. The pharmaceutical composition of claim 1, wherein the predetermined geometric shape has a surface area to volume ratio greater than a sphere.

10. (canceled)

11. The pharmaceutical composition of claim 1, wherein each particle of the plurality of micro and/or nanoparticles is substantially the same size and has substantially the same geometric shape.

12. The pharmaceutical composition of claim 1, wherein the particles of the plurality of nanoparticles have a polydispersity of about 0.003.

13. The pharmaceutical composition of claim 4, wherein the hydrophilic active pharmaceutical agent is a biologic.

14. The pharmaceutical composition of claim 1, wherein the composition of the particle may or may not be crosslinked.

15-21. (canceled)

22. The pharmaceutical composition of claim 1, wherein said protein is an antibody selected from the group consisting of abciximab, alemtuzumab, basiliximab, bevacizumab, cetuximab, daclizumab, eculizumab, ibritumomab tiuxetan, infliximab, muromonab-CD3, natalizumab, omalizumab, panitumumab, ranibizumab, rituximab, and traztuzumab.

23. A method of forming a plurality of monodisperse pharmaceutical composition particles comprising:
introducing a solution having at least 20 weight % protein into a plurality of cavities of a polymer mold, wherein the cavities have predetermined geometric shapes and a broadest dimension less than about 10 micrometers;
lyophilizing the aqueous solution within the cavities of the mold to form protein particles substantially corresponding to the shape of the mold cavity; and
removing the protein particles from the cavities of the mold;
wherein the protein particles substantially retain the geometric shape of the cavity for more than about four hours at about 37° C. in saline.

24. The method of claim 23, wherein the protein particles substantially retain the geometric shape of the cavity for more than about five hours at about 37° C. in saline.

25. The method of claim 23, wherein harvesting comprises removing the particles onto a harvesting sheet.

26. The method of claim 25, wherein the particles are arranged in an ordered array on the harvesting sheet, the ordered array mirroring an ordered array of the cavities of the mold.

27. The method of claim 23, wherein the solution has at least 50 weight % protein.

28. The method of claim 23, wherein the solution has at least 75 weight % protein.
